(19) United States
(12) Patent Application Publication
(10) Pub. No.: US 2005/0032696 A1
(43) Pub. Date: Feb. 10, 2005

(54) Muscle transcription factors

(76) Inventors: Edna Hardeman, Beecroft (AU); Nanthakumar Subramaniam, Wentworthville (AU); Kim Leonie Guven, Westmead (AU); Enoch Shao Ern Tay, Baulkham Hills (AU); Patsie Polly, Dover Heights (AU); Laura Laudi Issa, St. Peters (AU)

Correspondence Address: FROMMER LAWRENCE & HAUG, 745 FIFTH AVENUE- 10TH FL., NEW YORK, NY, 10151, US

(21) Appl. No.: 10/850,874

(22) Filed: May 21, 2004

Related U.S. Application Data
(63) Continuation-in-part of application No. PCT/AU02/01600, filed on Nov. 26, 2002.

(30) Foreign Application Priority Data
Nov. 27, 2001 (AU) PR9139

(51) Int. Cl. A61K 38/17; A61K 48/00; C07K 14/705; C07H 21/04

(52) U.S. Cl. 514/12; 514/44; 536/23.5; 530/350; 435/69.1; 435/320.1; 435/325

(57) ABSTRACT

Methods are provided for regulating muscle type by regulating the function of MusTRD polypeptides. Also provided are novel isoforms of the MusTRD gene.

### FIELD OF THE INVENTION

The present invention relates to methods of regulating muscle type by regulating the function of MusTRD polypeptides. The present invention also relates to novel isoforms from the MusTRD gene.

### BACKGROUND TO THE INVENTION

Adult skeletal muscle consists of two specialised cells: slow-twitch, fatigue-resistant, oxidative and fast-twitch, glycolytic myofibers. In the developing mouse embryo, cells that give rise to adult myofibers, the myoblasts, differentiate into adult phenotypes by two phases of differentiation and diversification. The first differentiation phase (7-8 dpc) gives rise to primary myotubes that go on to form slow myofibers. The second phase (12.5 dpc) gives rise to myotubes that contribute to secondary slow and presumptive fast myofibers. Myotube segregation into specialised fiber types beginning around 15 dpc when motor neuron connections are established, is driven by activation of specific programs of gene expression. Consequently, during late embryonic and postnatal development, expression of specific isoforms of thick and thin filament contractile proteins become restricted to prospective slow- or fast-twitch myofibers. In the final stage of peri- and postnatal muscle development myofibers undergo maturation and growth hypertrophy. In congenital myopathies and nerve injury the myofiber composition of skeletal muscle is disrupted either through aberrant myofiber maturation or myofiber conversion. Hence, the focus of our studies is to identify transcription factors that regulate myofiber diversification and myofiber type-specific gene expression.

We have previously cloned human muscle TFII-I repeat domain-containing protein-1α1 (hMusTRD1α1—previously called MusTRD1), from a human skeletal muscle cDNA library (O'Mahoney et al. 1998). Human MusTRD1α1 mRNA is predominant in human skeletal muscle and heart and was initially thought to code for a 458 amino acid protein. Subsequent identification of a sequencing error revealed an open reading frame that encodes a 944 amino acid protein.

hMusTRD11 contains five repeat domains (RD), each arranged in a helix-loop-helix (HLH) manner, that share approximately 70% amino-acid homology with those of the transcription factor TFII-I (Roy et al. 1997). TFII-I, which is expressed predominantly in liver and spleen, specifically targets Inr elements in the adenovirus ML and c-fos promoters. hMusTRD1α1, however, does not interact with TFII-I target elements (O'Mahoney et al. 1998). Both hMusTRD1α1 and TFII-I possess a leucine zipper in their extreme N-terminus, believed to be involved in heterodimerisation. However, hMusTRD1α1 also contains a myc-type HLH dimerisation motif between amino acids 458-466. hMusTRD contains two nuclear localisation signal (NLS) motifs between amino acids 407-413 (NLS1) and 883-889 (NLS2). In addition to direct interacting with lnr elements in its target genes, TFII-I interacts with other HLH factors, such as Burkitt's tyrosine kinase (Btk) and serum response factor (SRF), and responds to mitogen-activated factors such as c-Src and ERK1. Hence, hMusTRD1α1 and TFII-I may represent an emerging family of transcriptional regulators that integrate messages from multiple signalling pathways to coordinate gene expression in a cell-specific manner.

The gene encoding hMusTRD1α1 localises to chromosome 7q11.23 and is deleted in the multi-systemic disorder Williams-Beuren Syndrome (WS) that arises from a hemizygotic microdeletion spanning 18 genes.

WS is characterized by supravalvular aortic stenosis (SVAS), neurological and cognitive defects with a unique personality profile, infantile hypercalcemia, dental malformations, musculoskeletal anomalies and growth retardation with short stature. SVAS is attributed to the loss of the elastin (ELN) gene, while haploinsufficiency of the syntaxin 1A (STX1A) and LIM-kinase1 (LIMK) genes may be associated with the neurological and cognitive defects. The remainder of the phenotypes currently cannot be assigned to specific gene deletions. The musculoskeletal anomalies, including joint contractures, muscular pain and kyphoscoliosis, cause WS patients to lack stamina and fatigue easily. An underlying myopathy has been reported and may account for the physical limitations, however the disease causing gene/genes have not been identified. Furthermore, growth retardation is thought to be due to an underlying endocrine problem. WS patients are also reported to exhibit altered muscle myofiber composition and distribution (Voit et al. 1991).

### SUMMARY OF THE INVENTION

We have determined that the MusTRD gene is differentially expressed in different muscle tissues as different isoforms. These isoforms arise from differential splicing resulting in proteins in which the amino terminus is highly conserved and the carboxy terminus is highly variable through inclusion or exclusion of spliced exons. We have isolated 1 splice variant from human muscle and 11 splice variants from different mouse skeletal muscles and shown that different skeletal muscles express different combinations of MusTRD isoforms.

We have also identified two DNA binding domains (DBD) in hMusTRD1α1: DBD1 is located between amino acids 351-458 and DBD2 is located between amino acids 544-944.

To investigate the role of hMusTRD1α1 and related isoforms in skeletal muscle fiber development, we examined the regulatory potential of the normal and a 458aa truncated (Δ) peptide of hMusTRD1α1, named ΔhMusTRD1α1, common to all MusTRD isoforms. We have demonstrated that a polypeptide consisting of amino acids 1-458 of hMusTRDα1 is capable of occupying the hMusTRD1α1-binding motif on DNA, but fails to activate transcription, thus blocking functions of MusTRD isoforms. We demonstrate that both hMusTRD1α1 and ΔhMusTRD1α1 are capable of repressing the promoter/enhancer regions of slow fiber-specific genes, MHClslow and Tnlslow, but not the fast fiber gene MHCllbfast in muscle cell culture.

Using hMusTRD1α1 transgenic mouse models, we have shown that products from the MusTRD gene are required for at least three processes in myogenesis: 1) establishment of slow myofiber phenotype, 2) myofiber maturation and 3) myofiber growth hypertrophy. We have also identified various MusTRD splice variants in different muscles and at different developmental timepoints. In addition, distinct regions of the hMusTRD1α1 protein elicit different fiber-specific gene regulation programs in different skeletal muscles.

The identification of various MusTRD splice variants in different muscles together with the differential effect of a truncated version (amino acids 1-458) of hMusTRDα1 versus full-length hMusTRD1α1 on fiber phenotype in muscles of the crural block indicates that different isoforms will perform different functions with respect to fiber type determination in different muscles. Finally, disruption of MusTRD function recapitulates the growth and musculoskeletal defects found in WS, hence MusTRD is a candidate gene for these components of the WS phenotype.

Accordingly, the present invention provides a method of modulating the relative composition of slow and fast myofibers in muscle tissue of a human or animal which method comprises modulating in myogenic cells of the human or animal the levels and/or activity of MusTRD1.

The present invention also provides a method of modulating the relative composition of slow and fast myofibers in muscle tissue of a human or animal which method comprises administering to the human or animal a compound capable of modulating the levels and/or activity of MusTRD1 in myogenic cells of the human or animal.

The present invention further provides a method of modulating the amount of slow and/or fast myofibers in muscle tissue of a human or animal which method comprises modulating in myogenic cells of the human or animal the levels and/or activity of MusTRD1.

In another aspect, the present invention provides a method of modulating the amount of slow and/or fast myofibers in muscle tissue of a human or animal which method comprises administering to the human or animal a compound capable of modulating the levels and/or activity of MusTRD or an isoform thereof in myogenic cells of the human or animal.

The present invention also provides a method of regulating myofiber specialisation in a human or animal which method comprises modulating in myogenic cells of the human or animal the levels and/or activity of MusTRD or an isoform or fragment thereof.

The present invention further provides a method of regulating myofiber specialisation in a human or animal which method comprises administering to the human or animal a compound capable of modulating the levels and/or activity of a MusTRD polypeptide in myogenic cells of the human or animal.

In the above methods, it is preferred that the compound is a MusTRD polypeptide or fragment thereof, or a nucleic acid encoding said compound. More preferably, the compound is hMusTRD1α1, an orthologue thereof or a fragment thereof.

In another aspect, the present invention provides a method of treating a disease or condition characterised by muscular defects which method comprises administering to the human or animal a compound capable of modulating the levels and/or activity of a MusTRD polypeptide in myogenic cells of the human or animal.

The muscular defects may be abnormal myofiber composition, abnormal myofiber maturation and/or abnormal growth hypertrophy of differentiated myotubes.

Preferably, the compound is a MusTRD polypeptide or fragment thereof, or a nucleic acid encoding said compound.

We have also shown that a truncated MusTRD polypeptide inhibits expression of genes involved in the slow myogenic phenotype, such as expression of myosin light chain 1 slowA (MLC1slowA), α-tropomyosin slow (α-Tmslow), myosin heavy chain type I (MHC I), and troponin I slow (Tnlslow). Thus hMusTRD1α1 is involved in regulating expression of a number of genes required for the production of slow fibers.

Accordingly, the present invention provides a method of regulating expression of a myosin light chain 1 slowA (MLC1_{slowA}), α-tropomyosin slow (α-Tm_{slow}), myosin heavy chain type I (MHC I), and troponin I slow (TnI_{slow}) polypeptides in a cell which method comprises administering to/expressing in said cell a MusTRD polypeptide or fragment thereof.

Furthermore, we have also shown that MusTRD functions as a repressor of gene expression by inhibiting MEF2C-mediated transcriptional activation. Accordingly, the present invention also provides a method of inhibiting MEF2C-mediated gene expression in a cell by modulating the levels of MusTRD in said cell, such as by administering to/expressing in said cell a MusTRD polypeptide or fragment thereof.

The present invention further provides a polypeptide comprising a MusTRD polypeptide or fragment thereof, or a polynucleotide encoding the same, for use in (i) modulating the relative composition of slow and fast myofibers in muscle tissue of a human or animal; (ii) modulating the amount of slow and/or fast myofibers in muscle tissue of a human or animal; (iii) regulating myofiber specialisation in a human or animal; and/or (iv) treating muscular defects.

The present invention also relates to novel isoforms of the MusTRD gene, particularly splice variants. Accordingly, in another aspect, the present invention provides a polypeptide comprising the amino acid sequence shown in any one of SEQ ID Nos. 2, 4, 6, 8, 10, 12, 14, 16 and 18 or an orthologue thereof with the proviso that where the orthologue is a human orthologue, the full length human CREAM-1 polypeptide (959 amino acids), the full length human WBSCR11 polypeptide (944 amino acids), the full length human GTF2IRD1 polypeptide (944 amino acids) and the human GTF3 polypeptide are specifically excluded.

Preferred orthologues are human orthologues. Human orthologues include polypeptides having the same C-terminal exon splicing pattern as the corresponding mouse isoforms described herein, subject to the above disclaimer. Human orthologues include polypeptides encoded by any one of SEQ ID Nos. 24, 25 and 26.

The present invention also provides a human MusTRD polypeptide which comprises a Box 5 region and/or an RD5 region and fragments thereof which comprise a Box 5 region and/or an RD5 region.

In one embodiment, said polypeptide fragments of any of the above polypeptides comprise the transcriptional activation/repression domain of the full-length polypeptide.

In one embodiment, said polypeptide fragments comprise a DBD1 DNA binding domain and/or a DBD2 DNA binding domain.

The present invention also provides polynucleotides encoding said polypeptides. Further, the present invention provides a polynucleotide selected from the group consisting of:
(a) polynucleotides having the sequence as shown in any one of SEQ ID Nos. 1, 3, 5, 7, 9, 11, 13, 15, 17 and 19 and orthologues thereof
(b) fragments of the polynucleotides of (a) comprising a sequence encoding a Box 5 region and/or an RD5 region.
(c) fragments of the polynucleotides of (a) comprising a sequence encoding a DBD1 domain and/or a DBD2 domain.
(d) polynucleotides which are degenerate as a result of the genetic code to any of the polynucleotides of (a), (b) or (c); and
(e) polynucleotides which are complementary to the polynucleotides of (a), (b), (c) or (d);
   with the proviso that the full length human CREAM-1 nucleotide sequence, the full length human WBSCR11 nucleotide sequence, the full length human GTF2IRD1 nucleotide sequence and the full length human GTF3 nucleotide sequence are specifically excluded.

The present invention further provides a polynucleotide selected from the group consisting of:
(a) polynucleotides having the sequence as shown in any one of SEQ ID Nos. 1, 3, 5, 7, 9, 11, 13, 15, 17 and 19 and orthologues thereof
(b) fragments of the polynucleotides of (a) comprising a sequence encoding a Box 5 region and/or an RD5 region.
(c) fragments of the polynucleotides of (a) comprising a sequence encoding a DBD1 domain and/or a DBD2 domain.
(d) polynucleotides which are degenerate as a result of the genetic code to any of the polynucleotides of (a), (b) or (c); and
(e) polynucleotides which are complementary to the polynucleotides of (a), (b), (c) or (d);

The present invention further provides nucleic acid vectors comprising a polynucleotide of the invention, as well as host cells comprising a polynucleotide of the invention. The present invention also provides a method of producing a polypeptide of the invention which comprises culturing a host cell of the invention under conditions that allow for expression of said polypeptide in said cell.

The present invention further provides a transgenic non-human animal, which animal is transgenic by virtue of comprising a polynucleotide of the invention.

In another aspect, the present invention provides an antibody that binds specifically to a MusTRD polypeptide of the invention. In one embodiment, said antibody binds specifically to a MusTRD polypeptide of the invention comprising a Box 5 region or an RD5 region, or a DBD1 region or a DBD2 region.

Probes/primers based on regions of the MusTRD nucleotides which are differentially spliced may be used to detect different MusTRD isoforms in biological sample.

Accordingly the present invention provides a nucleotide probe/primer which hybridises specifically to a MusTRD polynucleotide sequence selected from exons 19, 21, 22, 23, 26, 27, 30 and 31 of a mouse MusTRD isoform, or the equivalent region of an orthologue thereof. Preferably the ortholologue is a human sequence.

In another embodiment, the present invention provides a nucleotide probe/primer which hybridises specifically to a MusTRD polynucleotide selected from a box 5 region and an RD5 region.

The present invention also provides a method of identifying the presence of a MusTRD isoform in a sample which method comprises determining the presence in the sample of one or more nucleotide regions selected from exons 19, 21, 22, 23, 26, 27, 30 and 31 of a mouse MusTRD isoform, or the equivalent region of an orthologue thereof. Preferably, the presence of the one or more nucleotide regions is determined by nucleotide amplification means, such as RT-PCR, using one or more primers of the invention.

The present invention also provides a method of identifying the presence of a MusTRD isoform in a sample which method comprises:
(a) providing an antibody of the invention;
(b) incubating the sample with said antibody under conditions which allow for the formation of an antibody-antigen complex; and
(c) determining whether an antibody-antigen complex comprising said antibody is formed.
   Preferably, the antibody binds specifically to a box5 region or an RD5 region of a MusTRD polypeptide.

### DETAILED DESCRIPTION OF THE INVENTION

Unless defined otherwise, all technical and scientific terms used herein have the same meaning as commonly understood by one of ordinary skill in the art (e.g., in cell culture, molecular genetics, nucleic acid chemistry, hybridization techniques and biochemistry). Standard techniques are used for molecular, genetic and biochemical methods (see generally, Sambrook et al., Molecular Cloning: A Laboratory Manual, 3rd ed. (2001) Cold Spring Harbor Laboratory Press, Cold Spring Harbor, N.Y. and Ausubel et al., Short Protocols in Molecular Biology (1999) 4^{th} Ed, John Wiley & Sons, Inc.—and the full version entitled Current Protocols in Molecular Biology, which are incorporated herein by reference) and chemical methods.

A. MusTRD Polypeptides

MusTRD polypeptides include hMusTRD1α1 and isoforms thereof, as well as orthologues thereof. MusTRD polypeptides also include allelic variants of any of the above. Full-length MusTRD polypeptides are typically polypeptides that have DNA binding activity (i.e. USE B1 motif binding activity as defined below) and comprise 5 or 6 TFII-I/MusTRD type repeat domains (RDs)..

The term isoform as used herein, refers to a naturally occurring variant of a polypeptide of interest which is naturally encoded either by the same gene as the polypeptide of interest (in which case the isoform typically differs from the polypeptide of interest due to differential RNA processing, such as splicing and/or post-translational processing) or by a different gene. Where the isoform is encoded by a different gene, the level of homology at the amino acid level will typically be very high, preferably at least 85, 90 or 95% overall identity. It is particularly preferred that isoforms have a high level of homology between DNA-binding domains (for example amino acids 351 to 458 and/or 544 to 944 of the hMusTRD1α1 sequence shown as SEQ ID No. 31, or the equivalent region in other MusTRD polypeptides), i.e. at least 85, 90 or 95% overall identity between DNA-binding domains.

The various specific isoforms referred to herein are named using the format MusTRDXα/βY. The name of the isoform is based on the peptide sequence. The first number, X, indicates the unique combination of C-terminal RDs present. Isoforms contain 1 of 2 possible C-terminal exons and these are designated “α”(exon 31) or “β” (exon 30). The final number, Y, indicates the specific combination of unique C-terminal exons present.

Orthologues are homologous polypeptides from another species that have an equivalent function in that species to the function that the polypeptide of interest or its isoforms perform in humans. By way of example, the mouse polypeptide BEN is an orthologue of hMusTRD1α1, whereas the ten newly identified mouse polypeptide sequences of the present invention shown as SEQ ID Nos 2, 4, 6, 8, 10, 12, 14, 16, 18 and 20 are isoforms of BEN.

Preferred orthologues are human orthologues. Human orthologues include polypeptides having the same C-terminal exon splicing pattern as the corresponding mouse isoforms described herein. The genomic sequence of the human MusTRD gene is known in the art and consequently, the polypeptide sequence of human orthologues having the same exon splicing pattern as the mouse isoforms described herein can be determined by reference to the genomic sequence and its exon/intron boundaries (see GenBank Accession No. NT 007867—sequence of human chromosome 7, which gives the location of MusTRD as nucleotides 548538..697334; also GenBank Accession Nos. AC004851 and AC005231). In particular, the intron/exon boundaries of the 27 exons which make up human Cream-1 are described in Table 1 of Yan et al., 2000. However, Cream-1 lacks exons corresponding to exons 23, 26, 27 and 30 of the mouse sequence. The nucleotide sequence for the human equivalents of exons 23 and 30 are given as SEQ ID Nos. 28 and 29.

The level of homology between orthologues will typically be lower than between isoforms. Consequently, the level of homology at the amino acid level will typically be at least 60, 75, 80 or 85% overall identity. However again, it is preferred that orthologues have a high level of homology between DNA-binding domains (for example amino acids 1 to 458 of the human MusTRD sequence shown as SEQ ID No. 31, or the equivalent region in other MusTRD polypeptides), i.e. at least 75, 80 or 85% overall identity between DNA-binding domains.

Sequence homology (such as sequence identity) can be calculated using a range of algorithms implemented using computer programs known in the art. These programs typically first generate an optimum alignment, taking into account appropriate gap penalties and using a scaled similarity score matrix. A suitable computer program for carrying out such an alignment is the GCG Wisconsin Bestfit package (University of Wisconsin, U.S.A.; Devereux eta/., 1984, Nucleic Acids Research 12:387). Examples of other software than can perform sequence comparisons include, but are not limited to, the BLAST package (see Ausubel et al., 1999 ibid—Chapter 18), FASTA (Atschul et al., 1990, J. Mol. Biol., 403-410) and the GENEWORKS suite of comparison tools. Both BLAST and FASTA are available for offline and online searching (see Ausubel et al., 1999 ibid, pages 7-58 to 7-60). However it is preferred to use the GCG Bestfit program (using the default gap penalty for amino acid sequences of −12 for a gap and −4 for each extension, and the public default matrix values).

Once the software has produced an optimal alignment, it is possible to calculate % homology, preferably % sequence identity. The software typically does this as part of the sequence comparison and generates a numerical result.

The amino acid sequence of hMusTRD1α1 is shown as SEQ ID. No. 31. The amino acid sequence of mouse BEN is shown as SEQ ID No.20.

In addition to the specific sequences of MusTRD polypeptides disclosed herein, additional isoforms and/or orthologues may be identified by, for example probing cDNA/genomic DNA libraries with nucleic acid probes designed using the amino acid/nucleotide sequences in any of SEQ ID Nos 1 to 26, or PCR using primers designed using said amino acid/nucleotide sequences.

Preferably orthologues are derived from mammalian cells such as primates, including humans, and domestic animals including pigs, cows, sheep, goats, horses and the like. They may also be derived from avian cells such as chicken, duck or goose cells, or fish cells.

Fragments of the above MusTRD polypeptides include fragments that contain at least a DNA binding domain that is capable of binding to a polynucleotide comprising a USE B1 motif. A USE B1 motif is defined as a polynucleotide sequence consisting of AGCCACAGGATTAA. The USE B1 motif and methods for determining binding of polypeptides to polynucleotides comprising a USE B1 motif are described further in O'Mahoney et al., 1998. Methods for assessing binding include electrophoretic mobility shift assays (EMSAs).

An example of a suitable DNA-binding fragment is a polypeptide consisting of amino acids 351 to 458 and 544 to 944 of hMusTRD (SEQ ID No. 31) or its equivalent in other MusTRD polypeptides. The minimal region required for DNA-binding activity may be smaller than 458 amino acids and can be determined by a person skilled in the art by progressively deleting amino acid sequence from the N-terminal and/or C-terminal end of the 458 amino acid region until binding to a USE B1 motif is substantially abolished. Analysis of the MusTRD sequence indicates that the DNA binding domains are from amino acids 408 to 420 and 738 to 765 and therefore preferred fragments comprise amino acids 408 to 420 and/or 738 to 765 of hMusTRD (SEQ ID No. 31) or their equivalent in other MusTRD polypeptides.

Other fragments include the regions of MusTRD polypeptides that interact with components of the transcriptional machinery to effect transcriptional regulation—herein termed a transactivation domain although such a domain may either activate or repress transcription depending on the context. The sequence of these transactivation domains differ in the various isoforms described herein due to differential splicing. By way of an example, a suitable fragment comprising a transactivation domain is from amino acid 459 to the C-terminus of human MusTRD (SEQ ID No. 31), or its equivalent in other MusTRD polypeptides.

It is particularly preferred that a MusTRD fragment derived from the C-terminus of a MusTRD polypeptide comprises a Box 5 region and/or an RD5 region. A Box 5 region is defined herein as a sequence consisting essentially of the sequence VKSRGSELHPNSVWPLPLPRAGPSTAPGTGRHWALRGTQPTTE GQAHPLVLPTR (SEQ ID No. 32)(the C-terminal 54 amino acids of five of the mouse isoforms shown in the sequence listings herein), or the equivalent sequence in other MusTRD polypeptides (including isoforms and orthologues). Preferably, a box 5 region comprises a contiguous region having at least 70, 80 or 90% sequence identity with SEQ ID. No. 32.

An RD5 region is defined herein as a sequence consisting essentially of the sequence RPVLVPYKLIRDSPDAVEVKGLPDDIPFRNPNTYDIHRLEKILKAREHV RMVIINQLQPF (SEQ ID No. 33), or the equivalent sequence in other MusTRD polypeptides (including isoforms and orthologues). Preferably, an RD5 region comprises a contiguous region having at least 70, 80 or 90% sequence identity with SEQ ID. No. 33.

Fragments of MusTRD polypeptides comprise at least 6, 8, 10, 12 or 15 contiguous amino acids, preferably at least 20, 30, 40 or 50 amino acids. Fragments also typically comprise fewer than 500, 400, 300 or 200 contiguous amino acids. Preferred fragments include those which include an epitope, more preferably those which are immunogenic.

Variants and derivatives of MusTRD polypeptides/fragments include any substitution of, variation of, modification of, replacement of, deletion of or addition of one (or more) amino acids from or to the sequence. In general, fewer than 20%, 10% or 5% (e.g. from 2, 3 or 5 to 10) of the amino acid residues of a variant or derivative are altered as compared with the original sequence, such as the corresponding region depicted in the sequence listings. Accordingly, the term “variant or derivative” does not encompass changes to the sequence such that the resulting polypeptide would no longer be recognisable to the skilled person as being a MusTRD polypeptide.

In one embodiment, it is preferred that the resultant amino acid sequence retains a biological activity of the original sequence (which, for example, may be DNA binding activity or transcriptional regulatory activity), preferably having at least 25 to 50% of an activity of the polypeptides presented in the sequence listings, more preferably at least substantially the same activity.

Thus, for example, amino acid substitutions may be made, for example from 1, 2 or 3 to 10, 20 or 30 substitutions provided that the modified sequence retains at least about 25 to 50% of, or substantially the same activity.

However, in an alternative embodiment, modifications to the amino acid sequences of a MusTRD polypeptide may be made intentionally to reduce a biological activity of the polypeptide. For example truncated polypeptides that remain capable of binding to target molecules but lack functional effector domains may be useful as inhibitors of the biological activity of the full-length molecule.

Amino acid substitutions may include the use of non-naturally occurring analogues, for example to increase blood plasma half-life of a therapeutically administered polypeptide (see below for further details on the production of peptide derivatives for use in therapy).

Conservative substitutions may be made, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar-uncharged | C S T M |
| | | N Q |
| | Polar-charged | D E |
| | | K R |
| AROMATIC | | H F W Y |

MusTRD polypeptides, including MusTRD polypeptides of the invention, are typically made by recombinant means, for example as described below. However they may also be made by synthetic means using techniques well known to skilled persons such as solid phase synthesis. Various techniques for chemically synthesising peptides are reviewed by Borgia and Fields, 2000, TibTech 18: 243-251 and described in detail in the references contained therein.

MusTRD polypeptides, including MusTRD polypeptides of the invention, may also be produced as fusion proteins, for example to aid in extraction and purification. Examples of fusion protein partners include glutathione-S-transferase (GST), hexahistidine, GAL4 (DNA binding and/or transcriptional activation domains) and β-galactosidase. It may also be convenient to include a proteolytic cleavage site between the fusion protein partner and the protein sequence of interest to allow removal of fusion protein sequences. Preferably the fusion protein will not hinder the activity of the MusTRD polypeptide to which it is linked.

In one embodiment, fragments of MusTRD polypeptides which comprise a DNA binding domain may be fused to a heterologous transcriptional regulatory domain such as a transcriptional activation domain or transcriptional repressor domain. In another related embodiment, MusTRD polypeptides which comprise a MusTRD transcriptional regulatory domain may be fused to a heterologous DNA binding domain.

MusTRD polypeptides may be in a substantially isolated form. It will be understood that the protein may be mixed with carriers or diluents which will not interfere with the intended purpose of the protein and still be regarded as substantially isolated. A MusTRD polypeptide may also be in a substantially purified form, in which case it will generally comprise the protein in a preparation in which more than 90%, e.g. 95%, 98% or 99% of the protein in the preparation is a MusTRD polypeptide.

Therapeutic Peptides

MusTRD polypeptides may be administered therapeutically to patients. It is preferred to use peptides that do not consist solely of naturally-occurring amino acids but which have been modified, for example to reduce immunogenicity, to increase circulatory half-life in the body of the patient, to enhance bioavailability and/or to enhance efficacy and/or specificity.

A number of approaches have been used to modify peptides for therapeutic application. One approach is to link the peptides or proteins to a variety of polymers, such as polyethylene glycol (PEG) and polypropylene glycol (PPG)—see for example U.S. Pat. Nos. 5,091,176, 5,214,131 and 5,264,209

Replacement of naturally-occurring amino acids with a variety of uncoded or modified amino acids such as D-amino acids and N-methyl amino acids may also be used to modify peptides

Another approach is to use bifunctional crosslinkers, such as N-succinimidyl 3-(2 pyridyidithio) propionate, succinimidyl 6-[3-(2 pyridyldithio) propionamido] hexanoate, and sulfosuccinimidyl 6-[3-(2 pyridyidithio) propionamido] hexanoate (see U.S. Pat. No. 5,580,853).

It may be desirable to use derivatives of MusTRD polypeptides that are conformationally constrained. Conformational constraint refers to the stability and preferred conformation of the three-dimensional shape assumed by a peptide. Conformational constraints include local constraints, involving restricting the conformational mobility of a single residue in a peptide; regional constraints, involving restricting the conformational mobility of a group of residues, which residues may form some secondary structural unit; and global constraints, involving the entire peptide structure.

B. MusTRD Polynucleotides

MusTRD polynucleotides comprise nucleic acid sequences encoding MusTRD polypeptides. It will be understood by a skilled person that numerous different polynucleotides can encode the same polypeptide as a result of the degeneracy of the genetic code. In addition, it is to be understood that skilled persons may, using routine techniques, make nucleotide substitutions that do not affect the polypeptide sequence encoded by the MusTRD polynucleotides to reflect the codon usage of any particular host organism in which the MusTRD polypeptides are to be expressed.

MusTRD polynucleotides may comprise DNA or RNA. They may be single-stranded or double-stranded. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the present invention, it is to be understood that the polynucleotides described herein may be modified by any method available in the art. Such modifications may be carried out in order to enhance the in vivo activity or life span of polynucleotides of the invention.

MusTRD polynucleotides also encompass nucleotide sequences that are capable of hybridising selectively to the sequences presented herein, or to the complement of any of the above. Nucleotide sequences are preferably at least 15 nucleotides in length, more preferably at least 20, 30, 40 or 50 nucleotides in length.

The term “hybridization” as used herein shall include “the process by which a strand of nucleic acid joins with a complementary strand through base pairing” as well as the process of amplification as carried out in polymerase chain reaction technologies.

MusTRD polynucleotides also include polynucleotides encoding MusTRD polypeptides which are capable of selectively hybridising to the nucleotide sequences presented herein, or to their complement. These polynucleotides will generally be at least 70%, preferably at least 80 or 90% and more preferably at least 95% or 98% homologous to the corresponding nucleotide sequences presented herein over a region of at least 20, preferably at least 25 or 30, for instance at least 40, 60 or 100 or more contiguous nucleotides.

The term “selectively hybridizable” means that the polynucleotide used as a probe is used under conditions where a target MusTRD polynucleotide is found to hybridize to the probe at a level significantly above background. The background hybridization may occur because of other polynucleotides present, for example, in the cDNA or genomic DNA library being screening. In this event, background implies a level of signal generated by interaction between the probe and a non-specific DNA member of the library which is less than 10 fold, preferably less than. 100 fold as intense as the specific interaction observed with the target DNA. The intensity of interaction may be measured, for example, by radiolabelling the probe, e.g. with ³²P.

Hybridization conditions are based on the melting temperature (Tm) of the nucleic acid binding complex, as taught in Berger and Kimmel (1987, Guide to Molecular Cloning Techniques, Methods in Enzymology, Vol 152, Academic Press, San Diego Calif.), and confer a defined “stringency” as explained below.

Maximum stringency typically occurs at about Tm−5° C. (5° C. below the Tm of the probe); high stringency at about 5° C. to 10° C. below Tm; intermediate stringency at about 10° C. to 20° C. below Tm; and low stringency at about 20° C. to 25° C. below Tm. As will be understood by those of skill in the art, a maximum stringency hybridization can be used to identify or detect identical polynucleotide sequences while an intermediate (or low) stringency hybridization can be used to identify or detect similar or related polynucleotide sequences.

In a preferred aspect, the MusTRD polynucleotides are nucleotide sequences that can hybridise to the nucleotide sequences shown in any of SEQ ID Nos 1, 3, 5, 7, 9, 11, 13, 15, 17 and 19 under stringent conditions (e.g. 65° C. and 0.1×SSC {1×SSC=0.15 M NaCl, 0.015 M Na₃ Citrate pH 7.0}).

MusTRD polynucleotides include both strands of the duplex, either individually or in combination.

Polynucleotides which are not 100% homologous to the sequences of the present invention but fall within the scope of the invention can be obtained in a number of ways. Other variants of the sequences described herein may be obtained for example by probing DNA libraries made from a range of individuals, for example individuals from different populations. In addition, other isoforms and/or orthologues may be obtained, and such isoforms and/or orthologues will in general be capable of selectively hybridising to the sequences shown in any of SEQ ID Nos 1, 3, 5, 7, 9, 11, 13, 15, 17 and 19. Such sequences may be obtained by probing cDNA libraries made from or genomic DNA libraries from other animal species, and probing such libraries with probes comprising all or part of said SEQ I.Ds under conditions of medium to high stringency.

Variants, isoforms and orthologues may also be obtained using PCR, such as degenerate PCR. The primers used in degenerate PCR will contain one or more degenerate positions and will be used at stringency conditions lower than those used for cloning sequences with single sequence primers against known sequences.

Alternatively, such polynucleotides may be obtained by site-directed mutagenesis of characterised sequences, such as any of SEQ ID Nos 1, 3, 5, 7, 9, 11, 13, 15 and 17. This may be useful where for example silent codon changes are required in sequences to optimise codon preferences for a particular host cell in which the polynucleotide sequences are being expressed. Other sequence changes may be desirable to introduce restriction enzyme recognition sites, or to alter the property or function of the polypeptides encoded by the polynucleotides.

MusTRD polynucleotides may be used to produce a primer, e.g. a PCR primer, a primer for an alterative amplification reaction, a probe e.g. labelled with a revealing label by conventional means using radioactive or non-radioactive labels, or the polynucleotides may be cloned into vectors. Such primers, probes and other fragments will be at least 15, preferably at least 20, for example at least 25, 30 or 40 nucleotides in length. Preferred fragments are less than 3000, 2000, 1000, 500 or 200 nucleotides in length.

Particularly preferred probes/primers are those based on regions of the MusTRD sequence which are differentially spliced in different isoforms. Use of such probes/primers will enable specific identification of different isoforms (as demonstrated in the Examples). Specific regions of interest are nucleotide probe/primers which hybridises specifically to a MusTRD polynucleotide sequence selected from exons 19, 21, 22, 23, 26, 27, 30 and 31 of a mouse MusTRD isoform, or the equivalent region of an orthologue thereof. Preferably the ortholologue is a human sequence. These primers/probes may be fragments of the mouse exon 19, 21, 22, 23, 26, 27, 30 or 31 sequences, or the complement thereof.

MusTRD polynucleotides such as a DNA polynucleotides and probes may be produced recombinantly, synthetically, or by any means available to those of skill in the art. They may also be cloned by standard techniques.

In general, primers will be produced by synthetic means, involving a step wise manufacture of the desired nucleic acid sequence one nucleotide at a time. Techniques for accomplishing this using automated techniques are readily available in the art.

Longer polynucleotides will generally be produced using recombinant means, for example using PCR (polymerase chain reaction) cloning techniques. This will involve making a pair of primers (e.g. of about 15 to 30 nucleotides) flanking a region of the sequence which it is desired to clone, bringing the primers into contact with mRNA or cDNA obtained from an animal or human cell, performing a polymerase chain reaction under conditions which bring about amplification of the desired region, isolating the amplified fragment (e.g. by purifying the reaction mixture on an agarose gel) and recovering the amplified DNA. The primers may be designed to contain suitable restriction enzyme recognition sites so that the amplified DNA can be cloned into a suitable cloning vector

C. Nucleotide Vectors

MusTRD polynucleotides, including MusTRD polynucleotides of the invention can be incorporated into a recombinant vector, typically a replicable vector. The vector may be used to replicate the nucleic acid in a compatible host cell. Suitable host cells include bacteria such as E. coli, yeast, mammalian cell lines and other eukaryotic cell lines, for example insect Sf9 cells.

Preferably, a MusTRD polynucleotide present in a vector is operably linked to a control sequence that is capable of providing for the expression of the coding sequence by the host cell,. i.e. the vector is an expression vector. The term “operably linked” means that the components described are in a relationship permitting them to function in their intended manner. A regulatory sequence “operably linked” to a coding sequence is ligated in such a way that expression of the coding sequence is achieved under condition compatible with the control sequences.

The control sequences may be modified, for example by the addition of further transcriptional regulatory elements to make the level of transcription directed by the control sequences more responsive to transcriptional modulators.

Vectors comprising MusTRD polynucleotides or other polynucleotides may be transformed or transfected into a suitable host cell as described below to provide for expression of a MusTRD polypeptide. This process may comprise culturing a host cell transformed with an expression vector as described above under conditions to provide for expression by the vector of a coding sequence encoding the protein, and optionally recovering the expressed protein.

The vectors may be for example, plasmid or virus vectors provided with an origin of replication, optionally a promoter for the expression of the said polynucleotide and optionally a regulator of the promoter. The vectors may contain one or more selectable marker genes, for example an ampicillin resistance gene in the case of a bacterial plasmid or a neomycin resistance gene for a mammalian vector. Vectors may be used, for example, to transfect or transform a host cell.

Control sequences operably linked to sequences encoding the MusTRD polypeptide include promoters/enhancers and other expression regulation signals. These control sequences may be selected to be compatible with the host cell for which the expression vector is designed to be used in. The term “promoter” is well-known in the art and encompasses nucleic acid regions ranging in size and complexity from minimal promoters to promoters including upstream elements and enhancers.

The promoter is typically selected from promoters which are functional in mammalian cells or other animal cells. Thus, the promoter is typically derived from promoter sequences of viral or eukaryotic genes. For example, it may be a promoter derived from the genome of a cell in which expression is to occur. With respect to eukaryotic promoters, they may be promoters that function in a ubiquitous manner (such as promoters of β-actin, tubulin) or, alternatively, a tissue-specific manner (such as the α-skeletal actin promoter). In the context of the therapeutic methods of the present invention, tissue-specific promoters specific for muscle cells are particularly preferred, for example the human α-skeletal actin promoter which expresses in fast muscle fibers and a combination of the human α-skeletal actin promoter plus Tnlslow upstream enhancer (USE) which expresses in all muscle fibers. They may also be promoters that respond to specific stimuli. Viral promoters may also be used, for example the Moloney murine leukaemia virus long terminal repeat (MMLV LTR) promoter, the rous sarcoma virus (RSV) LTR promoter or the human cytomegalovirus (CMV) IE promoter.

It may also be advantageous for the promoters to be inducible so that the levels of expression of the MusTRD polypeptide can be regulated during the life-time of the cell. Inducible means that the levels of expression obtained using the promoter can be regulated.

In addition, any of these promoters may be modified by the addition of further regulatory sequences, for example enhancer sequences. Chimeric promoters may also be used comprising sequence elements from two or more different promoters described above.

D. Host Cells

MusTRD polynucleotides and vectors comprising the same may be introduced into host cells for the purpose of replicating the vectors/polynucleotides and/or expressing MusTRD polypeptides. Although the MusTRD may be produced using prokaryotic cells as host cells, it is preferred to use eukaryotic cells, for example yeast, insect or mammalian cells. In particular, in the context of the methods of the invention, the polypeptides may be produced in the cells of the human or animal which it is desired to treat (target cells). Thus, typically the host cell will be a vertebrate cell, such as a mammalian, avian or fish cell. It is particularly preferred that the host cell/target cell is a muscle cell or a cell which can give rise to skeletal muscle such as satellite cells and stem cells from either embryonic or adult origin.

Vectors/polynucleotides may introduced into suitable host cells using a variety of techniques known in the art, such as transfection, transformation and electroporation. Where vectors/polynucleotides are to be administered to animals, several techniques are known in the art, for example infection with recombinant viral vectors such as retroviruses, herpes simplex viruses and adenoviruses, direct injection of nucleic acids and biolistic transformation.

E. Protein Expression and Purification

Host cells comprising MusTRD polynucleotides may be used to express MusTRD polypeptides. Host cells may be cultured under suitable conditions which allow expression of the MusTRD polypeptides. Expression of the proteins of the MusTRD polypeptides may be constitutive such that they are continually produced, or inducible, requiring a stimulus to initiate expression. In the case of inducible expression, protein production can be initiated when required by, for example, addition of an inducer substance to the culture medium, for example dexamethasone or IPTG.

If desired, MusTRD polypeptides can be extracted from host cells by a variety of techniques known in the art, including enzymatic, chemical and/or osmotic lysis and physical disruption.

F. Antibodies

The invention also provides monoclonal or polyclonal antibodies to MusTRD polypeptides of the invention or fragments thereof. Thus, the present invention further provides a process for the production of monoclonal or polyclonal antibodies to MusTRD polypeptides of the invention. In addition antibodies specific for MusTRD polypeptides in general may be used in the methods of the invention. In a preferred embodiment, the antibodies do not cross react with MusTRD polypeptides which lack a Box 5 region or an RD5 region. Preferred antibodies are also those which bind specifically to a Box 5 region or an RD5 region.

If polyclonal antibodies are desired, a selected mammal (e.g., mouse, rabbit, goat, horse, etc.) is immunised with an immunogenic polypeptide bearing a MusTRD epitope(s), such as a fragment containing a Box 5 region or an RD5 region. Serum from the immunised animal is collected and treated according to known procedures. If serum containing polyclonal antibodies to a MusTRD epitope contains antibodies to other antigens, the polyclonal antibodies can be purified by immunoaffinity chromatography. Techniques for producing and processing polyclonal antisera are known in the art. In order that such antibodies may be made, the invention also provides MusTRD polypeptides of the invention or fragments thereof haptenised to another polypeptide and their use as immunogens in animals or humans.

Monoclonal antibodies directed against MusTRD epitopes can also be readily produced by one skilled in the art. The general methodology for making monoclonal antibodies by hybridomas is well known. Immortal antibody-producing cell lines can be created by cell fusion, and also by other techniques such as direct transformation of B lymphocytes with oncogenic DNA, or transfection with Epstein-Barr virus. Panels of monoclonal antibodies produced against MusTRD epitopes can be screened for various properties; i.e., for isotype and epitope affinity.

An alternative technique involves screening phage display libraries where, for example the phage express scFv fragments on the surface of their coat with a large variety of complementarity determining regions (CDRs). This technique is well known in the art.

Antibodies, both monoclonal and polyclonal, which are directed against MusTRD epitopes may be useful in diagnosis and/or in therapeutic methods as described below.

Monoclonal antibodies, in particular, may also be used to raise anti-idiotype antibodies. Anti-idiotype antibodies are immunoglobulins which carry an “internal image” of an antigen of interest. Techniques for raising anti-idiotype antibodies are known in the art. These anti-idiotype antibodies may also be useful in therapy.

For the purposes of this invention, the term “antibody”, unless specified to the contrary, includes fragments of whole antibodies which retain their binding activity for a target antigen. Such fragments include Fv, F(ab′) and F(ab′)₂ fragments, as well as single chain antibodies (scFv) and single domain antibodies. Furthermore, the antibodies and fragments thereof may be humanised antibodies, for example as described in EP-A-239400.

MusTRD antibodies may be bound to a solid support and/or packaged into kits in a suitable container along with suitable reagents, controls, instructions and the like.

G. Methods of Identifying MusTRD Isoforms Antibodies of the invention may be used in method of detecting MusTRD polypeptides present in biological samples by a method which comprises: (a) providing an antibody specific for MusTRD; (b) incubating a biological sample with said antibody under conditions which allow for the formation of an antibody-antigen complex; and (c) determining whether an antibody-antigen complex comprising said antibody is formed.

Methods for determining the presence of antibody-antigen complexes are well known in the art and include techniques such as ELISA. Typically, the primary antibody is labelled or a secondary antibody is used which is labelled, for example conjugated to an enzyme.

Suitable samples generally include extracts from muscle tissue and other tissues that comprise muscle cells.

Probes/primers based on regions of the MusTRD nucleotides which are differentially spliced may be used to detect different MusTRD isoforms in biological samples.

Since the MusTRD isoforms arise from differential splicing, the techniques used to detect different isoforms will generally be based on detecting RNA in samples using methods such as Northern blotting or RT-PCR.

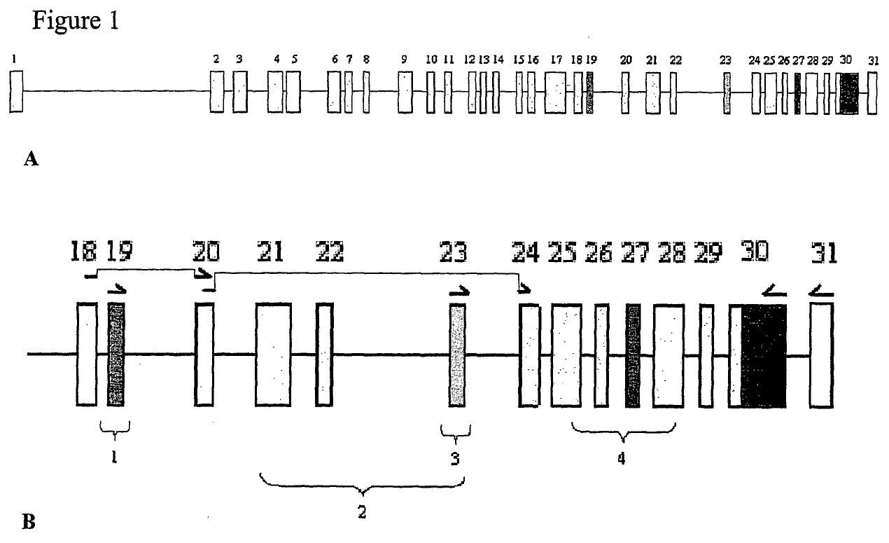

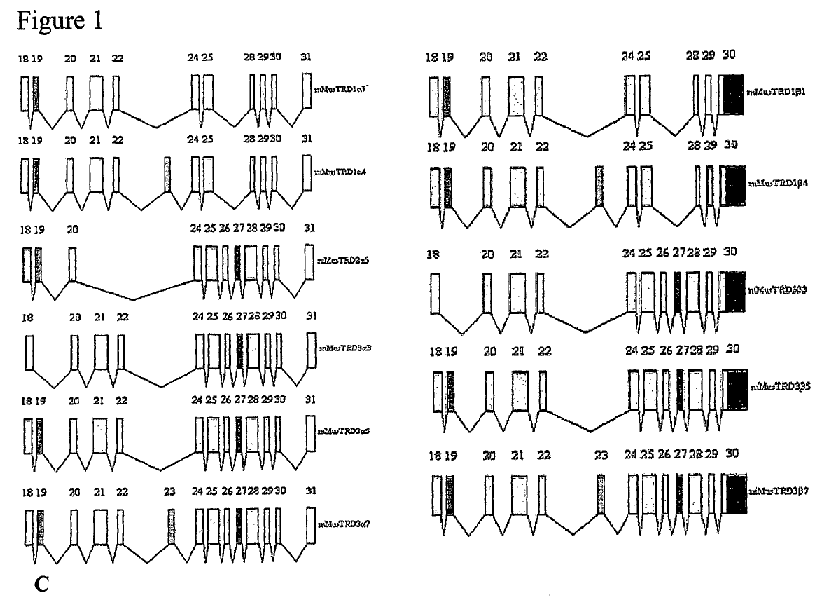

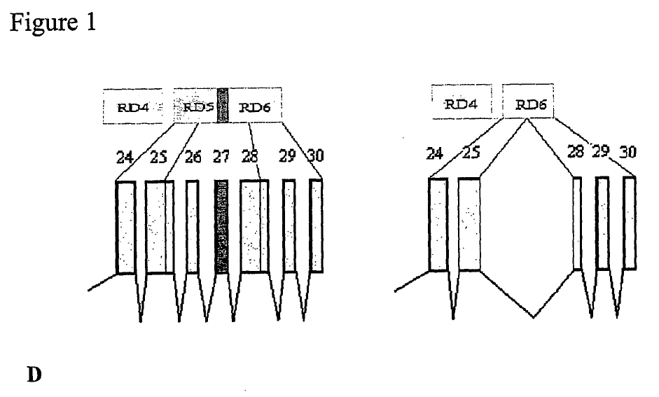

Total RNA can be extracted from the biological sample using techniques known in the art such as Trizol™ extraction.

Where probe hybridisation techniques, such as Northern blotting, are used, the probe should be selected such that substantially the entire probe sequence corresponds to a nucleotide sequence which is absent in at least one splice variant. FIG. 1 shows the splice pattern for the 11 isoforms identified. A probe which consists essentially of exon 23 sequences may be used to detect mouse MusTRD 1α4, 3α7, 1β4 and 3β7. Similarly, a probe which consists essentially of exon 31 sequences can be used to detect alpha isoforms whereas a probe which consists essentially of exon 30 sequences can be used to detect beta isoforms. Human exon 30 and exon 31 sequences which serve to detect different human isoforms are shown as SEQ ID Nos. 28 and 29.

Typically, a number of different probes will be used to identify specific isoforms based on the presence or absence of sequences which hybridise to the different probes. It may be desirable to include a control probe which hybridises specifically to the invariant N-terminal region encoded by exons 1 to 18 to confirm that the hybridising sequences present in the sample are MusTRD sequences.

It is preferred, however, to use amplification based detection techniques such as RT-PCR. When performing RT-PCR, typically at least one primer will be specific for an exon which is differentially spliced, such as exons 19, 21, 22, 23, 26, 27, 30 and 31 of a mouse MusTRD isoform, or the equivalent region of an orthologue thereof. Examples of suitable primers are given in the Examples, which were used to identify mouse isoforms in mouse tissue. The results of such detection techniques will typically be the presence or absence of amplification product.

Alternatively, using suitably designed primers that flank regions which are spliced differentially, detection can be based on the size of amplification product. Where an exon is not present in a splice variant between two regions to which the PCR primers hybridise, the amplification product will be smaller than when the exon is present.

H. Methods of Regulating Muscle Tissue Composition, Treating Muscular Disorders, and Predicting Athletic Performance.

We have shown that MusTRD plays an important role in regulating slow versus fast myogenic phenotype as well as in the maturation of myofibers and growth hypertrophy. We have also shown that different combinations of mouse (m) MusTRD isoforms are present in different muscles such as soleus, quadriceps and extensor digitorium longus (EDL) and in embryonic/foetal muscle fibers as represented by the C2C12 skeletal muscle cell line.

Accordingly, modulation of the activity of hMusTRD1α1 and its isoforms may be used to regulate the growth of slow and fast fiber types in muscle tissue. For example, modulation of MusTRD activity may be used to enhance the amount of slow muscle fibers in one or more muscles of an individual. Alternatively, modulation of MusTRD activity may be used to reduce the amount of slow muscle fibers in one or more muscles of an individual. The effect of modulating MusTRD activity in a muscle cell will generally depend on the muscle cell type. Further, if specific MusTRD isoforms are targeted, then the effect will depend on the particular MusTRD isoforms.

In a similar manner, modulation of the activity of hMusTRD1α1 and its isoforms may be used to modulate the relative composition of slow and fast fiber types in muscle tissue of an individual. Typically, this is achieved by stimulating the growth of one fiber type whilst repressing the growth of the other fiber type. It could also be achieved by conversion of one type to another.

Thus, in one embodiment, there is an increase in expression of one or more myosin heavy chain isoforms and other contractile protein genes specific to slow fibers and a decrease in expression of one or more myosin heavy chain isoforms and other contractile protein genes specific to fast fibers. In another embodiment, there is an increase in expression of one or more myosin heavy chain isoforms and other contractile protein genes specific to fast fibers and a decrease in expression of one or more myosin heavy chain isoforms and other contractile protein genes specific to slow fibers.

Modulation of the activity of hMusTRD1α1 and its isoforms may also be used to regulate, typically enhance, the maturation of myofibers.

Further, modulation of the activity of hMusTRD1α1 and its isoforms may also be used to regulate muscle growth (particularly stimulate muscle growth), for example to increase muscle mass. This is a consequence of its role in growth hypertrophy of differentiated myotubes. In particular, modulation of the activity of hMusTRD1α1 and its isoforms may be used to increase or decrease the cross-sectional area of a muscle of interest (preferably increase) and/or increase or decrease the fiber diameter (preferably increase).

Regulation of muscle growth and slow/fast fiber composition and amount via hMusTRD1α1 and its isoforms has a number of applications, including therapeutic applications. For example, patients who have experienced nerve damage resulting in fast fiber predominance and fiber atrophy may be treated to stimulate growth of slow fibers, to convert fast to slow fibers, and to promote growth hypertrophy. Also, individuals who experience the deleterious effects of normal ageing arising from a decrease in numbers of slow fibers, an increase in the number of fast fibers and fiber atrophy fiber, may be treated to increase the number of slow fibers and reverse atrophy. In another example, in diseases such as, but not limited to, human myopathies in which fast or slow fiber predominance is observed and/or fiber atrophy occurs, patients may be treated to either increase fast or slow fiber numbers to reverse the initial trend and to reverse fiber atrophy. Modulation of MusTRD activity may also be used to treat or prevent muscle degeneration.

Non-therapeutic applications include use in domestic animals, including cattle, sheep, pigs, chickens etc., to improve the quality of the muscles. In one embodiment, this may be achieved by generating transgenic animals that express a MusTRD polypeptide, or a fragment thereof. Production of transgenic animals is described below. In another non-therapeutic application, MusTRD transcript or protein abundance or distribution in muscle biopsies could be used as indicators of presence of muscle disease, performance for athletes and racehorses, and meat quality in beef cattle, sheep and pigs. The results from such tests would be used in diagnosis for disease and to assess the potential of the athlete or animal. The results from such tests would be used to design training regimens to achieve maximal performance, and to identify animal breeding stock that would produce meat with consistent, desirable qualities. MusTRD activity may be regulated typically by modulating the amount of MusTRD polypeptide in a cell and/or by modulating the activity of MusTRD polypeptide in the cell.

The amount of MusTRD polypeptide in a cell may be increased by introducing a polynucleotide into the cell that directs expression of a MusTRD polypeptide in that cell. The amount of MusTRD polypeptide in a cell may be decreased by introducing a polynucleotide into the cell which blocks expression of a MusTRD polypeptide, such as an antisense RNA, ribozyme or other inhibitory RNA sequence. It is preferred that the expression of the polynucleotide is limited to muscle cells by the use of an appropriate muscle-cell specific transcriptional regulatory sequence. It may also be desirable to include transcriptional regulatory control elements that are inducible and responsive to a compound not normally found in the subject organism such that expression can be induced by administering the compound to the subject.

The amount of MusTRD polypeptide in a cell may also be modulated by administering a compound to the cell that modulates expression of a MusTRD polypeptide, for example a compound which blocks transcription from a MusTRD gene.

The activity of MusTRD polypeptides in a cell may be modulated in a number of ways. For example, compounds may be administered which bind directly to MusTRD (such as antibodies) and prevent its interaction with other components of the cellular transcriptional machinery. Other compounds may not bind to MusTRD polypeptides but may compete directly with MusTRD for binding to target molecules. For example, a truncated MusTRD polypeptide which lacks a transcriptional activation domain, but has a functional DNA-binding domain, may bind to a USE-B1 sequence in a gene promoter but will be unable to activate/repress transcription (as shown in the Examples).

Suitable compounds in addition to antibodies and MusTRD mutant polypeptides may be identified using, for example, the assays described below.

We have also shown that a truncated MusTRD polypeptide can either inhibit or activate the expression of genes involved in the slow myogenic phenotype, such as expression of myosin light chain 1 slowA (MLC1_{slowA}), α-tropomyosin slow (α-Tm_{slow}), myosin heavy chain type I (MHC I), and troponin I slow (TnI_{slow}), depending on the specific muscle. Thus MusTRD isoforms are involved in regulating expression of a number of genes required for the production of slow fibers.

We have also shown that MusTRD functions as a repressor of gene expression by inhibiting MEF2C-mediated transcriptional activation. Accordingly, MusTRD polypeptides and polynucleotides encoding the same can be used in methods of inhibiting MEF2C-mediated -gene expression in a cell, such as by increasing the levels of a MusTRD polypeptide in said cell. Preferably the MusTRD polypeptide is a hMusTRD1α1 polypeptide or orthologue thereof.

1. Assays

The present invention provides assays that are suitable for identifying substances that modulate MusTRD activity. Such assays may be in vitro or in vivo.

Candidate Substances

A substance that modulates MusTRD activity as a result of an interaction with MusTRD polypeptides may do so in several ways. It may directly disrupt the binding of MusTRD polypeptide to a cellular component by, for example, binding to MusTRD polypeptide and masking or altering the site of interaction with the other component. Candidate substances of this type may conveniently be preliminarily screened by in vitro binding assays as, for example, described below and then tested, for example in a whole cell assay or in vivo assay. Examples of candidate substances include antibodies that recognise MusTRD polypeptides.

A substance which can bind directly to a MusTRD polypeptide may also inhibit its function in cellular transcription by altering its subcellular localisation thus preventing the MusTRD polypeptide from entering the cell nucleus. This can be tested using, for example, the whole cells assays described below. Non-functional homologues of MusTRD polypeptide may also be tested since they may compete with MusTRD polypeptide for binding to MusTRD recognition sites in promoters and/or binding to other components of the transcriptional machinery whilst being incapable of the normal functions of the MusTRD polypeptide. Such non-functional homologues may include naturally occurring MusTRD polypeptide mutants and modified MusTRD polypeptide sequences or fragments thereof. In particular, fragments of MusTRD polypeptide which comprise the DNA binding domain but lack other functional domains may be used to compete with full-length MusTRD polypeptide for binding to promoter regions.

Alternatively, instead of regulating the association of MusTRD with other cellular components directly, the substance may regulate, typically suppress, the biologically available amount of MusTRD polypeptide. This may be by inhibiting expression of the MusTRD, for example at the level of transcription, transcript stability, translation or post-translational stability. An example of such a substance would be antisense RNA or double-stranded interfering RNA sequences which suppresses the amount of MusTRD polypeptide mRNA biosynthesis.

Suitable candidate substances include peptides, especially of about 5 to 30 or 10 to 25 amino acids in size, based on the sequence of the various domains of MusTRD polypeptides described in section A, or variants of such peptides in which one or more residues have been substituted. Peptides from panels of peptides comprising random sequences or sequences which have been varied consistently to provide a maximally diverse panel of peptides may be used.

Suitable candidate substances also include antibody products (for example, monoclonal and polyclonal antibodies, single chain antibodies, chimeric antibodies and CDR-grafted antibodies) which are specific for MusTRD polypeptides. Furthermore, combinatorial libraries, peptide and peptide mimetics, defined chemical entities, oligonucleotides, and natural product libraries may be screened for activity as modulators of MusTRD activity. The candidate substances may be used in an initial screen in batches of, for example 10 substances per reaction, and the substances of those batches which show inhibition tested individually.

MusTRD Polypeptide Binding Assays

One type of assay for identifying substances that bind to MusTRD polypeptide involves contacting a MusTRD polypeptide, which is immobilised on a solid support, with a non-immobilised candidate substance determining whether and/or to what extent the MusTRD polypeptide and candidate substance bind to each other. Alternatively, the candidate substance may be immobilised and the MusTRD polypeptide non-immobilised.

In a preferred assay method, the MusTRD polypeptide is immobilised on beads such as agarose beads. Typically this is achieved by expressing the component as a GST-fusion protein in bacteria, yeast or higher eukaryotic cell lines and purifying the GST-fusion protein from crude cell extracts using glutathione-agarose beads. As a control, binding of the candidate substance, which is not a GST-fusion protein, to the immobilised MusTRD polypeptide may be determined in the absence of the MusTRD polypeptide. The binding of the candidate substance to the immobilised MusTRD polypeptide is then determined. This type of assay is known in the art as a GST pulldown assay. Again, the candidate substance may be immobilised and the MusTRD polypeptide non-immobilised.

It is also possible to perform this type of assay using different affinity purification systems for immobilising one of the components, for example Ni-NTA agarose and histidine-tagged components.

Binding of the MusTRD polypeptide to the candidate substance may be determined by a variety of methods well known in the art. For example, the non-immobilised component may be labelled (with for example, a radioactive label, an epitope tag or an enzyme-antibody conjugate). Alternatively, binding may be determined by immunological detection techniques. For example, the reaction mixture can be Western blotted and the blot probed with an antibody that detects the non-immobilised component. ELISA techniques may also be used.

Candidate substances are typically added to a final concentration of from 1 to 1000 nmol/ml, more preferably from 1 to 100 nmol/ml. In the case of antibodies, the final concentration used is typically from 100 to 500 μg/ml, more preferably from 200 to 300 μg/ml.

Whole Cell Assays

Candidate substances may also be tested on whole cells for their effect on MusTRD expression and/or activity. The candidate substances may have been identified by the above-described in vitro methods. Alternatively, rapid throughput screens for substances capable of modulating MusTRD expression and/or activity may be used as a preliminary screen.

The candidate substance, i.e. the test compound, may be administered to the cell in several ways. For example, it may be added directly to the cell culture medium or injected into the cell. Alternatively, in the case of polypeptide candidate substances, the cell may be transfected with a nucleic acid construct which directs expression of the polypeptide in the cell. The expression of the polypeptide may be under the control of a regulatable promoter.

Typically, an assay to determine the effect of a candidate substance on MusTRD expression comprises administering the candidate substance to a cell and determining whether the levels of MusTRD polypeptide and/or mRNA are affected using techniques such as Western blotting, Southern blotting and/or quantitative PCR.

The concentration of candidate substances used will typically be such that the final concentration in the cells is similar to that described above for the in vitro assays.

In vivo Assays

Candidate substances will ultimately need to be tested in an animal model to determine whether an effect on MusTRD activity/expression leads to an effect on the regulation of muscle fiber type/composition and/or growth.

A candidate substance may, for example, be administered to young animals, such as mice less than 4 weeks old. Administration may be performed as described in section H below. The animals are then monitored over a period of time, such as 2 to 12 months and samples taken of muscle tissue from one or more muscles of the animal to determine the amount of slow and fast muscle present.

As described in the Examples, this may typically be performed by measuring the expression of markers specific to different fiber types—such as myosin heavy chain (MHC) type-I for slow fibers and MHC-IIA, -IX, or -IIB for fast-twitch fibers. Muscles to be tested may include the soleus muscle (a slow-twitch muscle, 45% MHC-I_{slow} and 55% MHC-IIA_{fast}), the Extensor Digitorum Longus (EDL; a predominantly fast twitch muscle—80% MHC-IIB_{fast}, 15% MHC-IIA_{fast} and 5% MHC-Islow). Preferred candidate substances are those that result in at least a 20, 30, 40 or 50% change in expression of one or more myosin heavy chain isoforms specific to one fiber type. Thus, in one embodiment, there is an increase in expression of one or more myosin heavy chain isoforms specific to slow fibers and a decrease in expression of one or more myosin heavy chain isoforms specific to fast fibers. In another embodiment, there is an increase in expression of one or more myosin heavy chain isoforms specific to fast fibers and a decrease in expression of one or more myosin heavy chain isoforms specific to slow fibers.

Other tests include measuring muscle cross-sectional area and/or fiber diameter.

J. Administration

MusTRD polypeptides, fragments thereof and substances identified by the assay methods described above are preferably be combined with various components to produce compositions. Preferably the compositions are combined with a pharmaceutically acceptable carrier or diluent to produce a pharmaceutical composition (which may be for human or animal use). Suitable carriers and diluents include isotonic saline solutions, for example phosphate-buffered saline. The composition of the invention may be administered by direct injection. The composition may, for example, be formulated for parenteral, intramuscular, intravenous, subcutaneous, oral or transdermal administration.

Typically, in the case of polypeptides, each polypeptide may be administered at a dose of from 0.01 to 30 mg/kg body weight, preferably from 0.1 to 10 mg/kg, more preferably from 0.1 to 1 mg/kg body weight.

Polynucleotides/vectors encoding polypeptide components for use in the methods of the invention or inhibitory RNAs may be administered directly as a naked nucleic acid construct. When the polynucleotides/vectors are administered as a naked nucleic acid, the amount of nucleic acid administered may typically be in the range of from 1 μg to 10 mg, preferably from 100 μg to 1 mg.

Uptake of naked nucleic acid constructs by eukaryotic cells, such as mammalian cells is enhanced by several known transfection techniques for example those including the use of transfection agents. Example of these agents include cationic agents (for example calcium phosphate and DEAE-dextran) and lipofectants (for example lipofectam™ and transfectam™). Typically, nucleic acid constructs are mixed with the transfection agent to produce a composition.

Preferably the polynucleotide or vector is combined with a pharmaceutically acceptable carrier or diluent to produce a pharmaceutical composition. Suitable carriers and diluents include isotonic saline solutions, for example phosphate-buffered saline. The composition may be formulated, for example, for parenteral, intramuscular, intravenous, subcutaneous, oral or transdermal administration.

The routes of administration and dosages described are intended only as a guide since a skilled practitioner will be able to determine readily the optimum route of administration and dosage for any particular patient and condition.

K. Production of Transgenic Animals

Techniques for producing transgenic animals are well known in the art. A useful general textbook on this subject is Houdebine, Transgenic animals—Generation and Use (Harwood Academic, 1997)—an extensive review of the techniques used to generate transgenic animals from fish to mice and cows.

Advances in technologies for embryo micromanipulation now permit introduction of heterologous DNA into, for example, fertilised mammalian ova. For instance, totipotent or pluripotent stem cells can be transformed by microinjection, calcium phosphate mediated precipitation, liposome fusion, retroviral infection or other means, the transformed cells are then introduced into the embryo, and the embryo then develops into a transgenic animal. In a highly preferred method, developing embryos are infected with a retrovirus containing the desired DNA, and transgenic animals produced from the infected embryo. In a most preferred method, however, the appropriate DNAs are microinjected into pro-nuclear stage eggs by standard methods. Injected eggs are then cultured before transfer into the oviducts of pseudopregnant recipients and allowed to develop into mature transgenic animals. These techniques as well known. See reviews of standard laboratory procedures for microinjection of heterologous DNAs into mammalian fertilised ova, including Hogan et al., Manipulating the Mouse Embryo, (Cold Spring Harbor Press 1986); Krimpenfort et al., Bio/Technology 9:844 (1991); Palmiter et al., Cell, 41: 343 (1985); Kraemer et al., Genetic manipulation of the Mammalian Embryo, (Cold Spring Harbor Laboratory Press 1985); Hammer et al., Nature, 315: 680 (1985); Wagner et al., U.S. Pat. No. 5,175,385; Krimpenfort et al., U.S. Pat. No. 5,175,384, the respective contents of which are incorporated herein by reference.

Transgenic animals may also be produced by nuclear transfer technology as described in Schnieke, A. E. et al., 1997, Science, 278: 2130 and Cibelli, J. B. et al., 1998, Science, 280: 1256. Using this method, fibroblasts from donor animals are stably transfected with a plasmid incorporating the coding sequences for a binding domain or binding partner of interest under the control of regulatory sequences. Stable transfectants are then fused to enucleated oocytes, cultured and transferred into female recipients.

Analysis of animals that may contain transgenic sequences would typically be performed by either PCR or Southern blot analysis following standard methods.

By way of a specific example for the construction of transgenic mammals, such as cows, nucleotide constructs comprising a sequence encoding a binding domain fused to GFP are microinjected using, for example, the technique described in U.S. Pat. No. 4,873,191, into oocytes which are obtained from ovaries freshly removed from the mammal. The oocytes are aspirated from the follicles and allowed to settle before fertilisation with thawed frozen sperm capacitated with heparin and prefractionated by Percoll gradient to isolate the motile fraction.

The fertilised oocytes are centrifuged, for example, for eight minutes at 15,000 g to visualise the pronuclei for injection and then cultured from the zygote to morula or blastocyst stage in oviduct tissue-conditioned medium. This medium is prepared by using luminal tissues scraped from oviducts and diluted in culture medium. The zygotes must be placed in the culture medium within two hours following microinjection.

Oestrous is then synchronised in the intended recipient mammals, such as cattle, by administering coprostanol. Oestrous is produced within two days and the embryos are transferred to the recipients 5-7 days after oestrous. Successful transfer can be evaluated in the offspring by Southern blot.

Alternatively, the desired constructs can be introduced into embryonic stem cells (ES cells) and the cells cultured to ensure modification by the transgene. The modified cells are then injected into the blastula embryonic stage and the blastulas replaced into pseudopregnant hosts. The resulting offspring are chimeric with respect to the ES and host cells, and nonchimeric strains which exclusively comprise the ES progeny can be obtained using conventional cross-breeding. This technique is described, for example, in WO91/10741.

Aspects of the present invention will now be described with reference to the following Examples, which are illustrative only and non-limiting. The Examples refer to Figures:

### DETAILED DESCRIPTION OF THE FIGURES

FIG. 1: (A) Schematic representation of mMusTRD gene (GTF2IRD1) on chromosome 5, showing relative positions of introns. (B) Schematic representation of exons 18-31 of mMusTRD gene showing the forward primers targeting exon 19, exon 23, the coding sequence flanking exon 19, the coding sequence flanking exon 23 and reverse primers targeting exon 30 and exon 31. Also shown are the four alternatively spliced cassettes (1-4) and the mutually exclusively spliced exons (30 and 31) marked with an *. (C) Schematic representations of the alternative splicing events in the 3′ region of the mouse GTF2IRD1 gene (exons 18-31) that give rise to the 11 mMusTRD isoforms. (D) Schematic representation of cassette 4, which constitutes the 5′ region of RD5 and the 3′ region of RD6 in isoforms 2α5, 3α3, 3β3, 3α5, 3α7, 3β5 and 3β7, and the exons that form RD6 in isoforms 1α1, 1α4, 1β1 and 1β4 when this cassette is removed by alternative splicing.

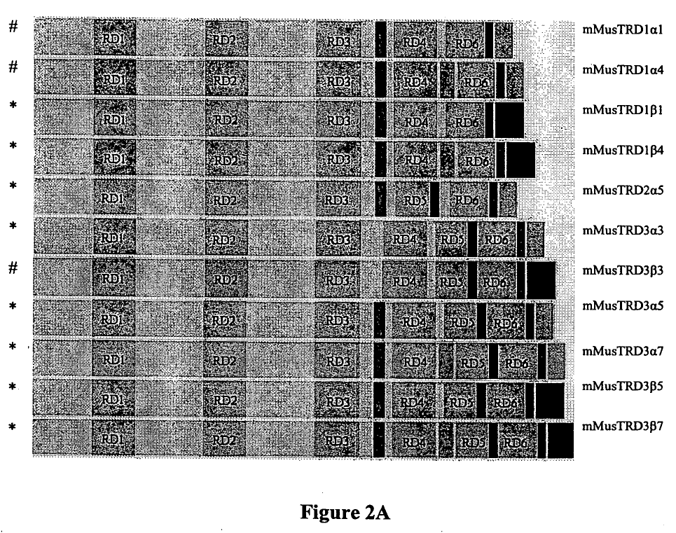

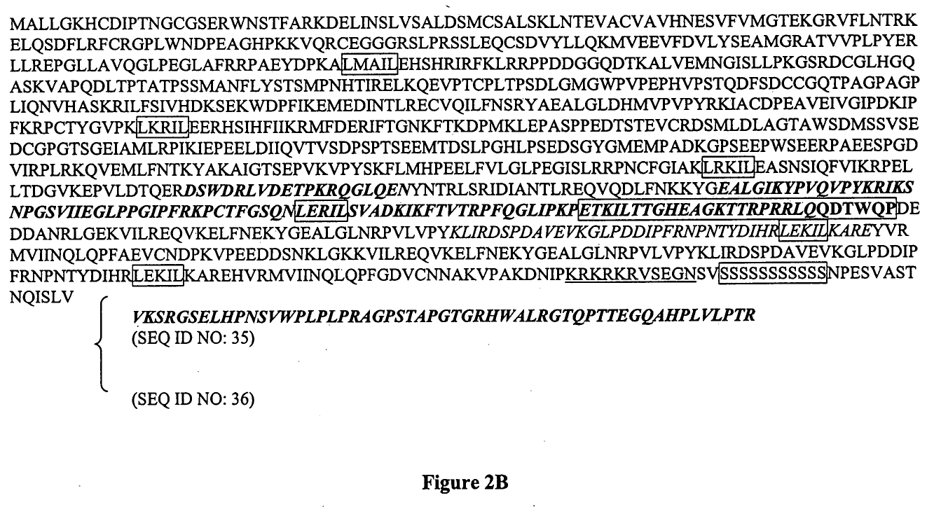

FIG. 2: (A) Schematic diagram of 11 mMusTRD isoforms. Completely sequenced isoforms are indicated *, while isoforms with sequenced 3′ regions indicated #. Repeat domains are shown in dark yellow, of which RD4, the 3′ half of RD5 and the 5′ half of RD6 are alternatively spliced, polyserine tract in red and nuclear localisation sequences in dark blue, signature boxes arising from alternative splicing of exons 19, 23, 31 and the 3′ region of exon 30 are shown in green, jade, pink and purple, respectively. (B) Amino acid sequence of MusTRD arising from all possible coding sequences. Peptide sequences arising from alternatively spliced cassettes are italicised and bold, in addition, the alternatively spliced, mutually exclusive 5′ region of exon 30 and exon 31 are in brackets. The amino acid sequence coded by exon 23 is also boxed to distinguish it from the larger cassette. Also shown are the nuclear localisation sequences (underlined), the polyserine tract (boxed) and 6 putative coregulator LXXIL motifs (boxed).

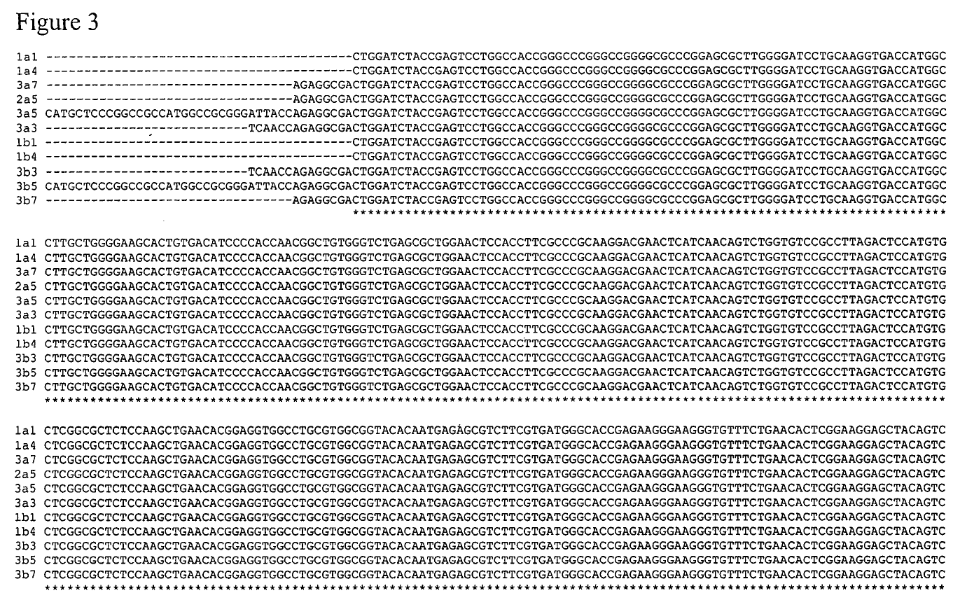

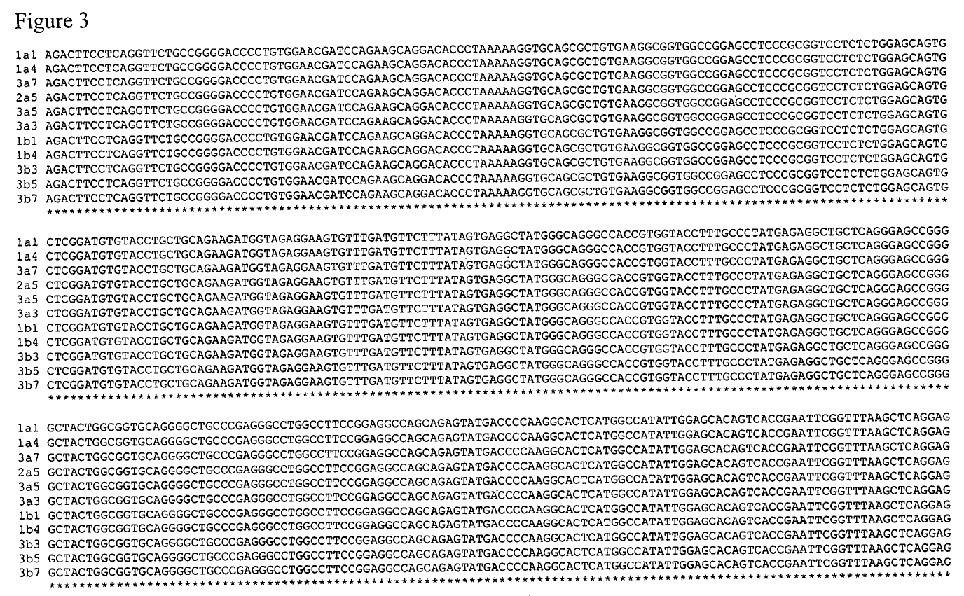

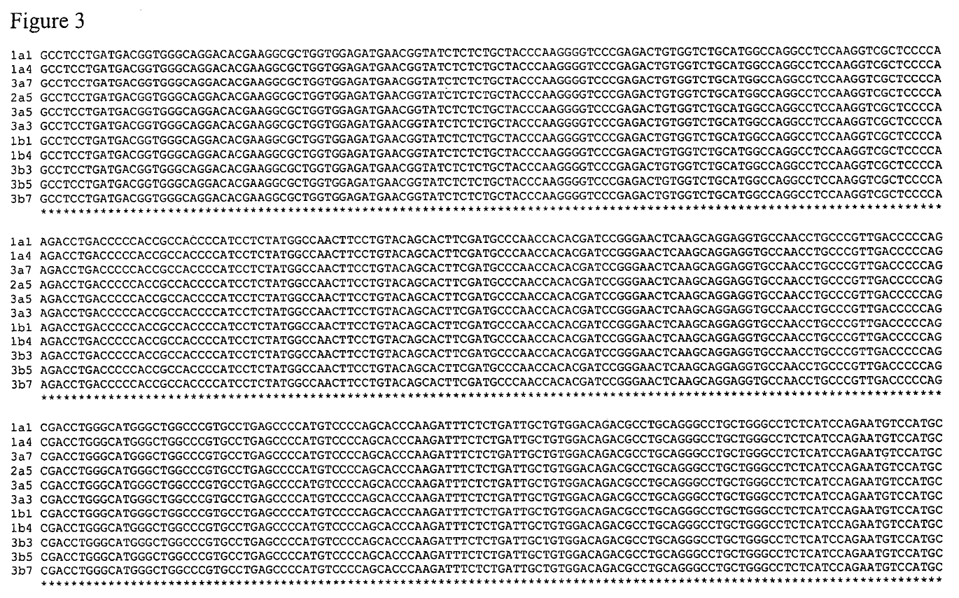

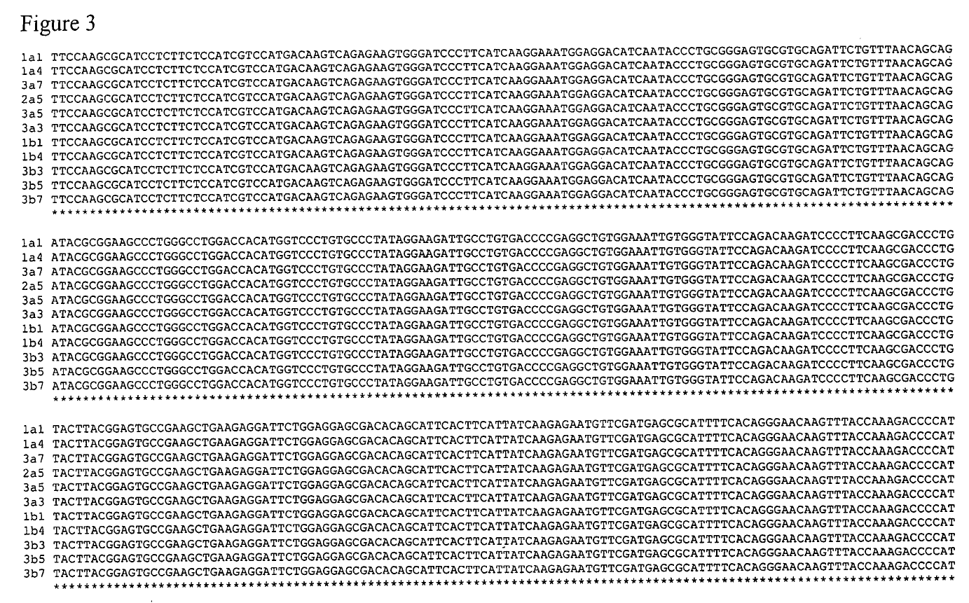

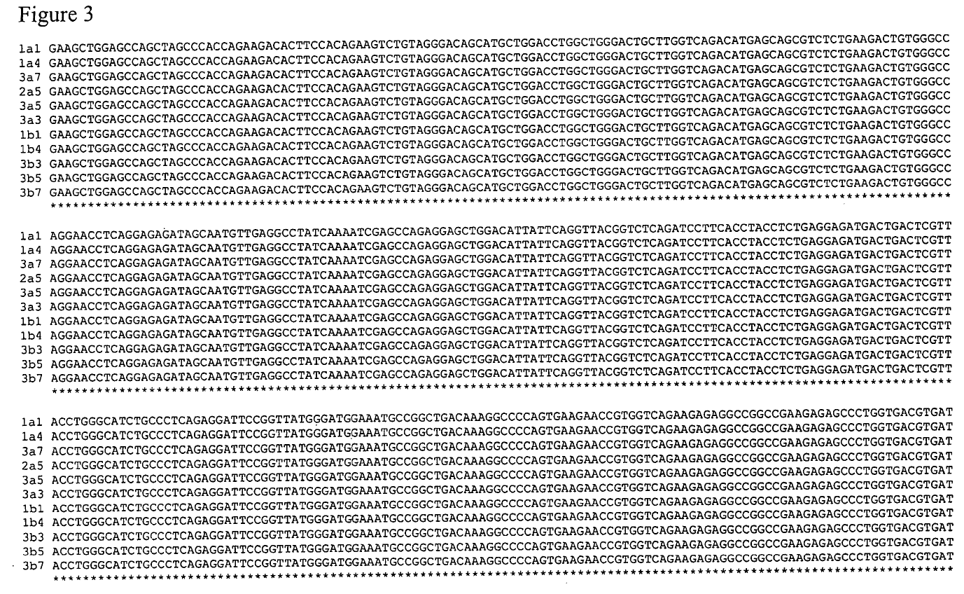

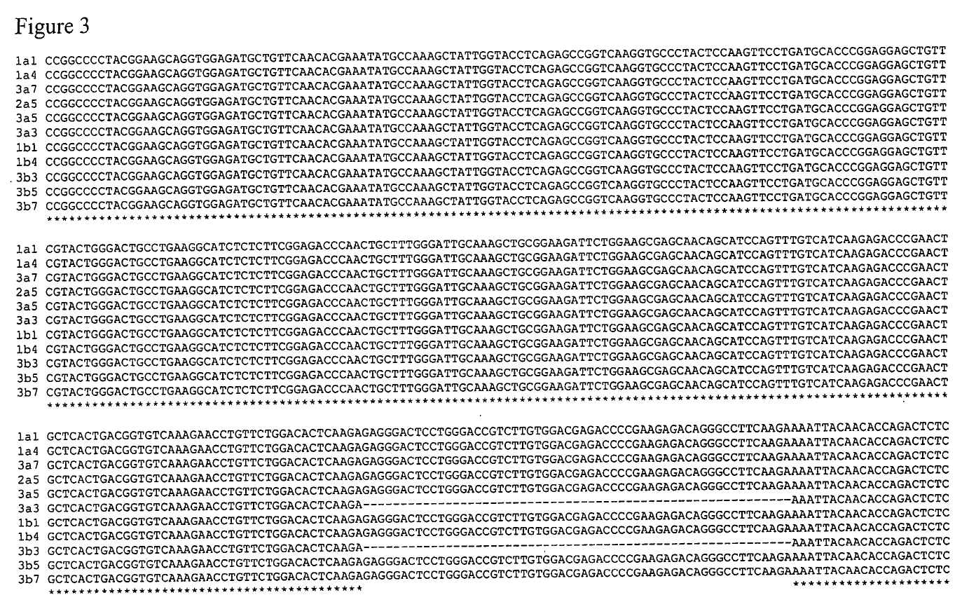

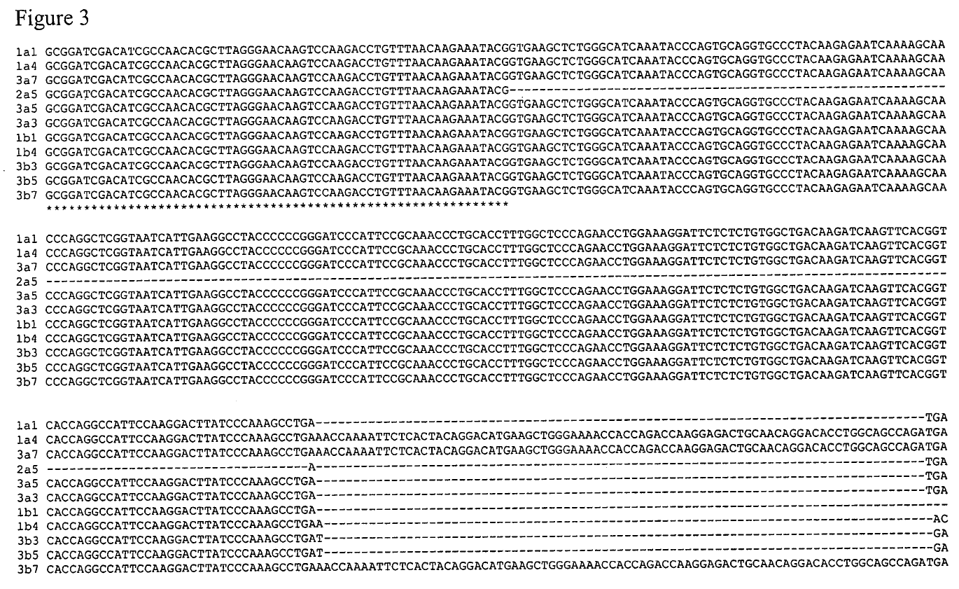

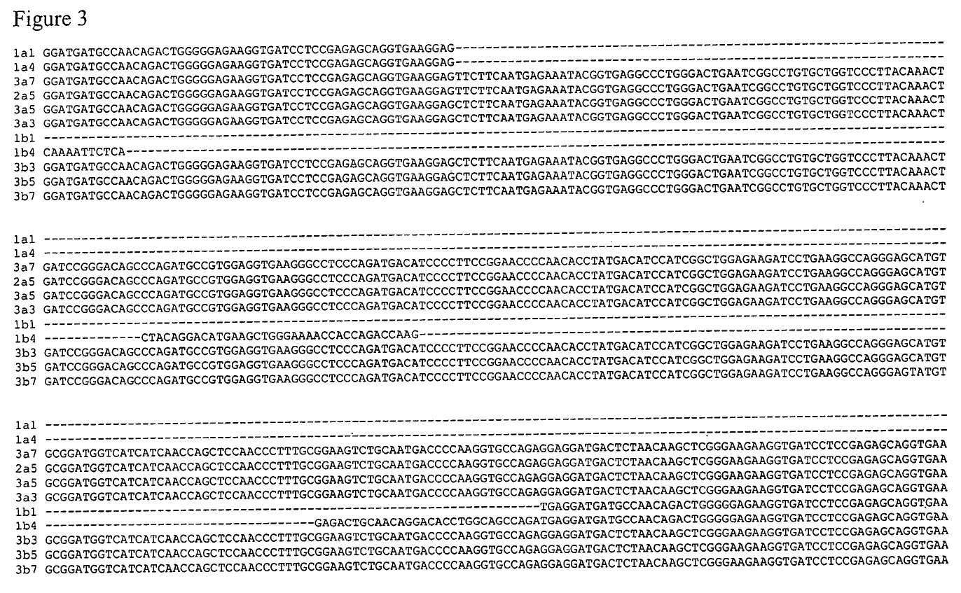

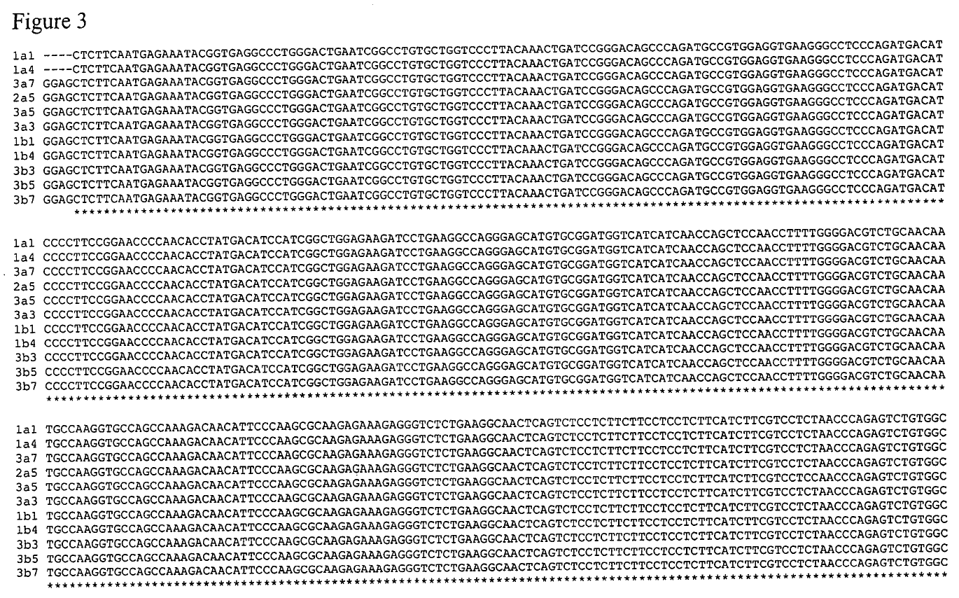

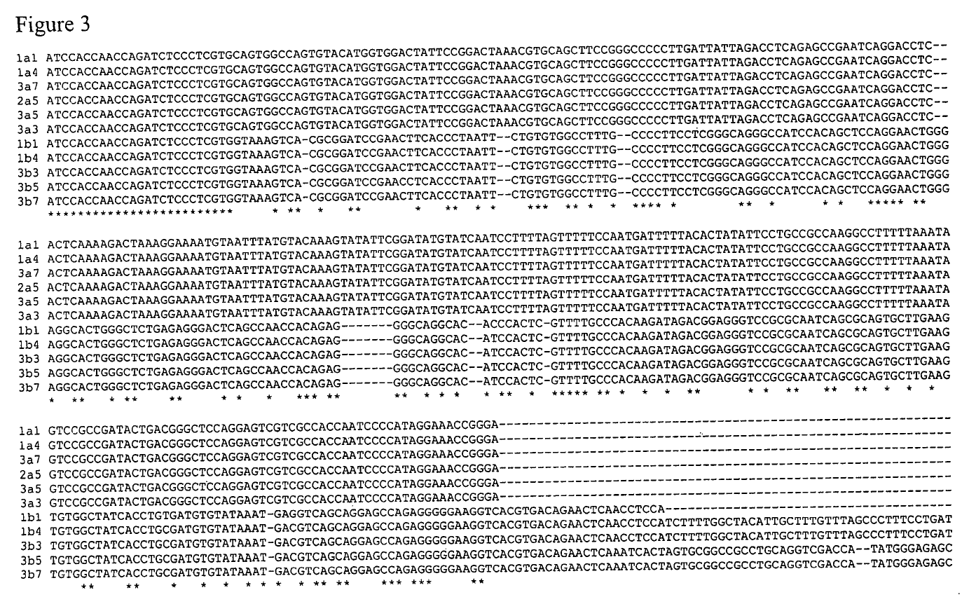

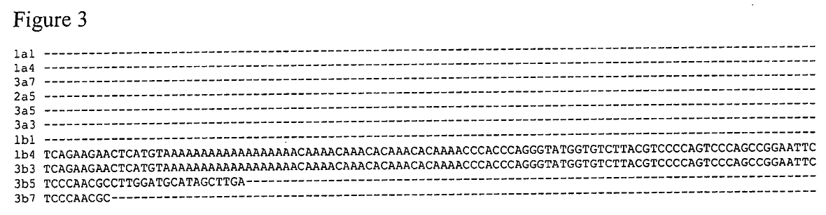

FIG. 3. Alignment of the nucleotide sequences of 11 mouse MusTRD isoforms.

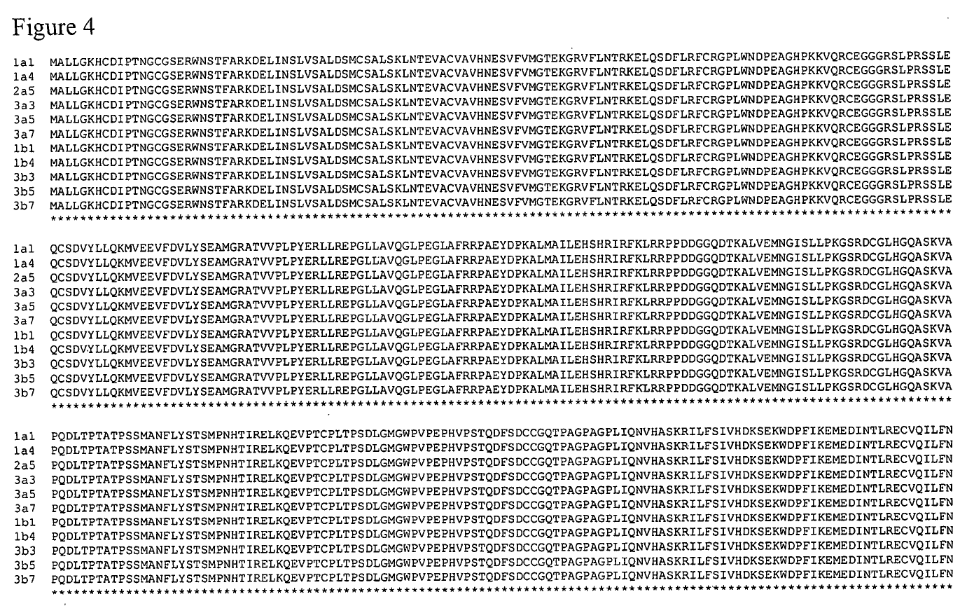

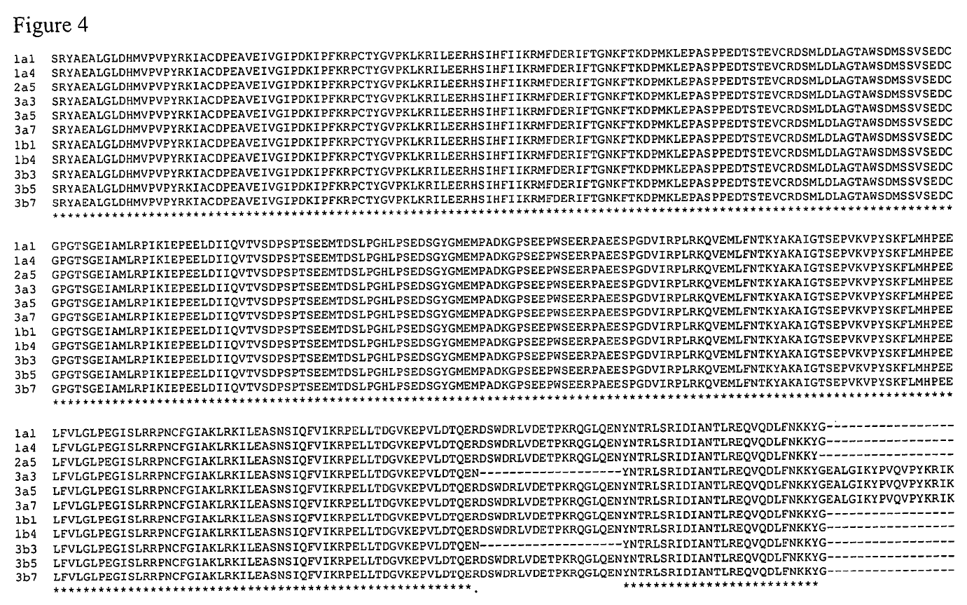

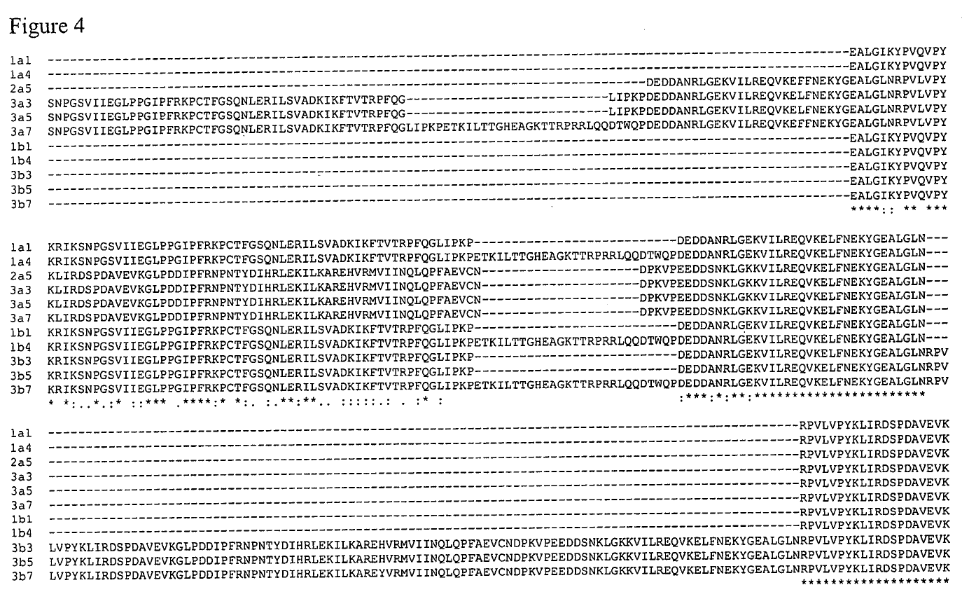

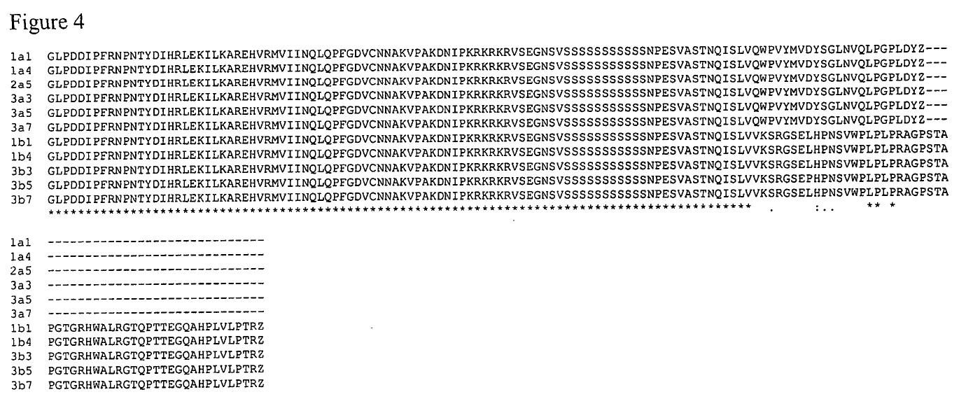

FIG. 4. Alignment of the amino acid sequences of 11 mouse MusTRD isoforms.

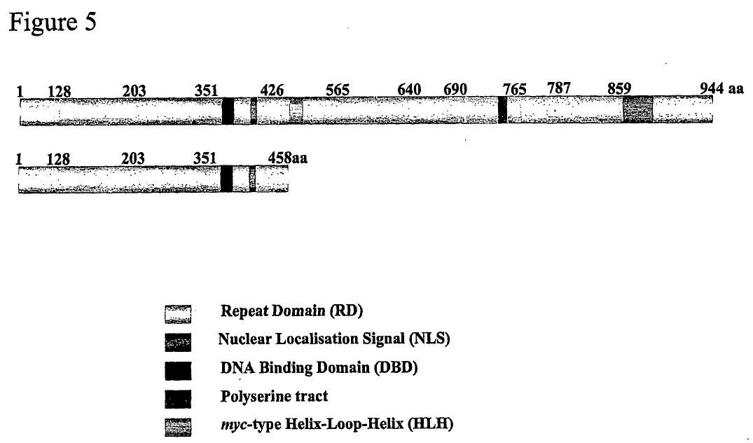

FIG. 5. Domain organisation of hMusTRD1α1. hMusTRD1α1₉₄₄ₐₐ and hMusTRD1α1₄₅₈ₐₐ are 944aa and 458aa in length and contain five and two TFII-I-like repeated domain structures of approximately 86-95aa (dark yellow), respectively. Amino acid alignments and structural motif searches of hMusTRD1α1 has revealed nuclear localisation signals (NLS; jade), a myc-helix-loop-helix (HLH; red), a polyserine tract (dark green) and two putative DNA binding domains (DBDs; blue).

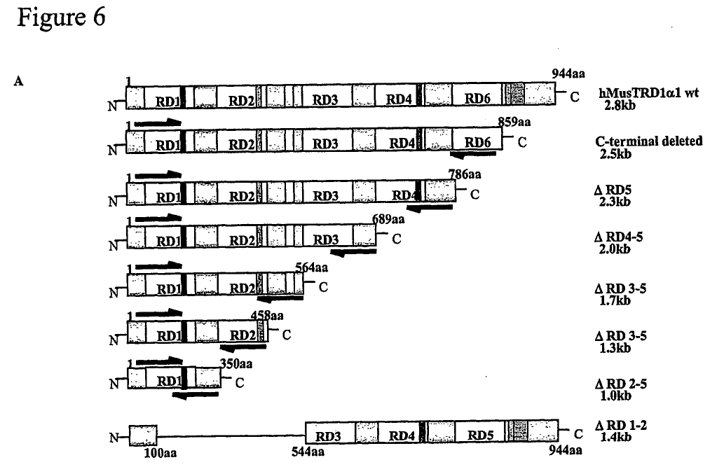

FIG. 6. The C-terminally and N-terminally truncated hMusTRD1α1 series. (A) C-terminal and N-terminal truncated versions of hMusTRD1α1 were generated by progressive removal of each repeat domain (RD) starting at the carboxy end of the molecule, in addition to an N-terminally deleted hMusTRD1α1 lacking RDs 1 and 2. A ladder of C-terminally truncated cDNAs were generated by PCR (B) and subcloned into the pcDNA3.1 myc/his expression vector (Invitrogen) for (C) in vitro translation of C-terminally deleted versions of hMusTRD1α1 to be used in subsequent functional analyses.

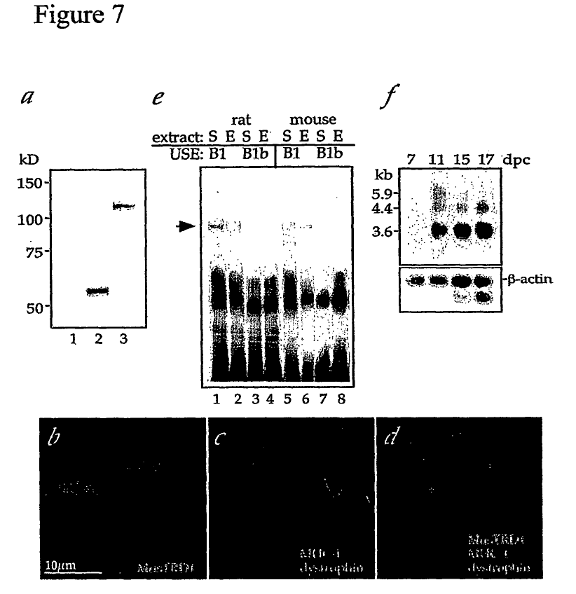

FIG. 7. Expression of MusTRD in mouse skeletal muscle. (A) Western blot analysis of full-length (lane 3) and truncated human MusTRD1α1 (amino acids 1-458; ΔhMusTRD1α1; lane 2) ectopically expressed in COS-7 cells. Untransfected COS-7 cell lysate is shown in lane 1. Immunofluorescent staining (FITC) of mouse soleus muscle revealed MusTRD is expressed in the nuclei (B) of both slow and fast myofibers (C). (C) Anti-myosin heavy chain type I (MHC-I_{slow}) staining distinguishes slow- (top) from fast-twitch (bottom) myofibers. Anti-dystrophin immunostaining delineates the plasma membrane of the muscle fiber (arrow). (D) Merge of (B) & (C) showing MusTRD expressing nuclei are within the plasma membrane of myofibers. (E) Detection of Tnl_{slow}-USE-B1 binding proteins in mouse muscle. Electromobility shift analysis of mouse soleus (S) and EDL (E) nuclear protein extracts and comparison with rat muscle nuclear extracts. B1 oligonucleotide contains the intact Inr-like binding site for hMusTRD1β1; B1b contains the 3 bp mutation that prevents binding. (F) Northern blot analysis of mouse MusTRD expression during mouse embryonic development. Three bands of 5.9, 4.4 and 3.6 kb are detected. (dpc=days post coitum).

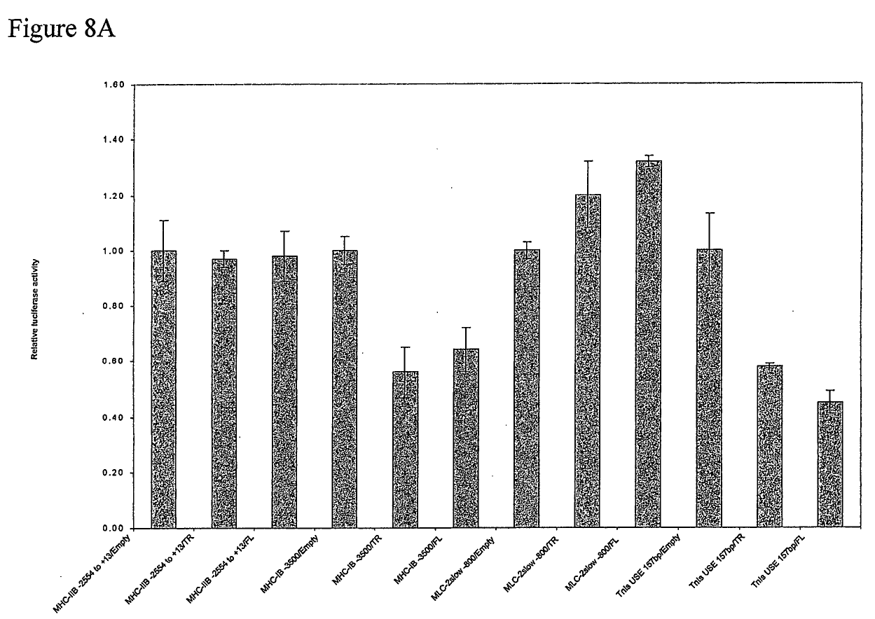

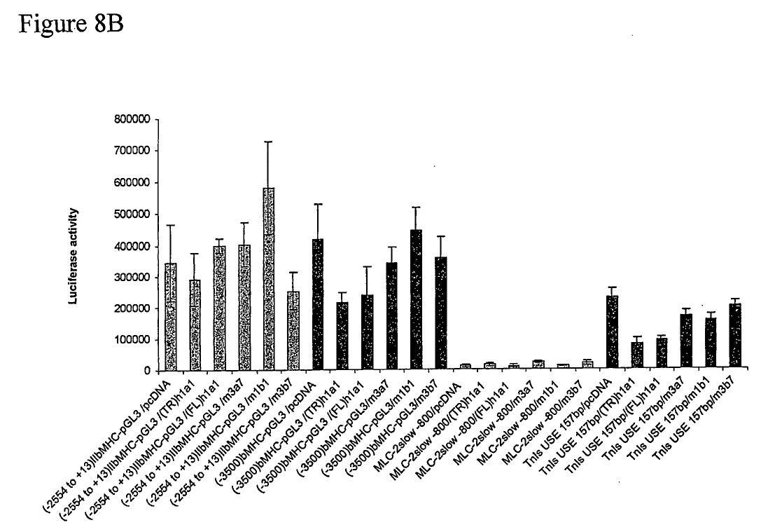

FIG. 8A. Regulation of slow and fast fiber-specific promoters by hMusTRD1α1 and ΔhMusTRD1α1 in C2C12 cells. hMusTRD1α1 or ΔhMusTRD1α1 expression plasmids were co-transfected with expression plasmids containing either −2554 to +13 of the MHIIB gene, −3500 to +462 of the MHCl_{slow} (MHCIB) gene, −800 to +12 of the MLC2_{slow} gene, or the 157 bp Tnl_{slow} enhancer linked to luciferase. Luciferase activity was determined in lysates from 3 different transfection cultures per plasmid combination. MusTRD-mediated repression or activation of transcriptional activity is expressed as fold difference from the basal/empty construct activity that was set at 1. Values shown represent the mean±standard deviation from three separate experiments. TR=ΔhMusTRD1α1, FL=hMusTRD1α1.

FIG. 8B. Regulation of slow and fast fiber-specific promoters by mMusTRD isoforms in C2C12 cells. mMusTRD3α7, 1,β1 or 3β7 expression plasmids were co-transfected with expression plasmids containing either −2554 to +13 of the MHIIB gene, −3500 to +462 of the MHCl_{slow} (MHCIB) gene, −800 to +12 of the MLC2_{slow} gene, or the 157 bp Tnlslow enhancer linked to luciferase. Luciferase activity was determined in lysates from 3 different transfection cultures per plasmid combination. MusTRD-mediated repression or activation of transcriptional activity is expressed as the actual light units. Values shown represent the mean±standard deviation from three separate experiments.

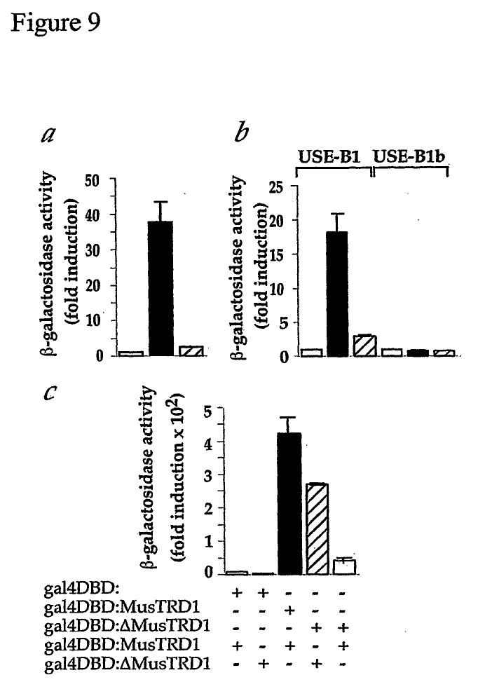

FIG. 9. Transactivation and dimerisation function of hMusTRD1α1 and ΔhMusTRD1α1. (A) hMusTRD1α1 (closed bars), but not ΔhMusTRD1α1 (hatched bars), exhibits autologous transactivation function, when fused to the gal4 DNA binding domain (gal4DBD). (B) In YM4271 yeast one-hybrid cells, wild-type (wt) hMusTRD1α1 (closed bars) and ΔhMusTRD1α1 (hatched bars) bound the Tnl_{slow}USE-B1 sequence, but not the USE-B1b sequence containing a mutated binding site. Open bars represent basal β-galactosidase reporter activity. (C) Both hMusTRD1α1 (closed bars) and ΔhMusTRD1α1 (hatched bars) homodimerize in yeast, but heterodimerization between the two proteins is less efficient (stippled bar). These data are expressed relative to control strains, and represent the mean fold induction in β-galactosidase activity of three independent experiments, each performed in triplicate.

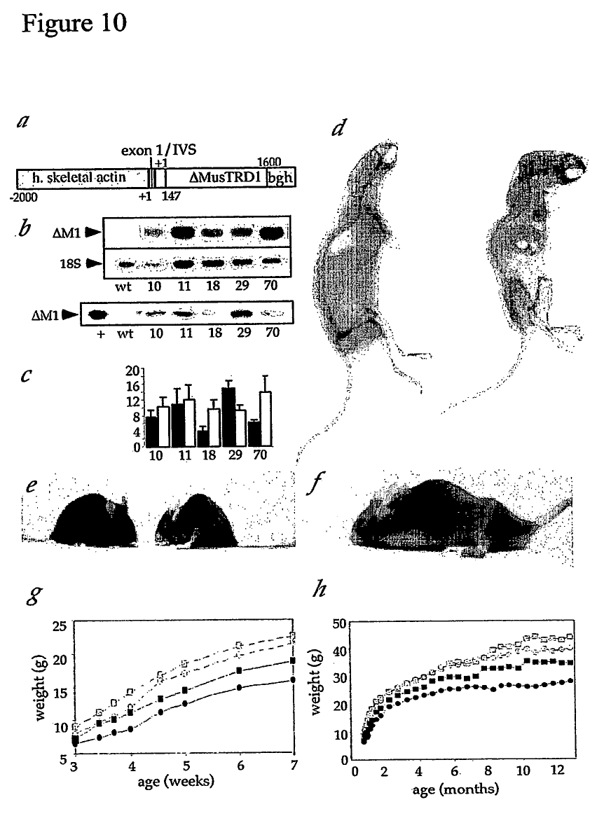

FIG. 10. Generation of ΔhMusTRD1α1 transgenic mice. (A) The −2000HSA:ΔhMusTRD1α1 transgenic construct contains the 2.2 kb Hind III fragment of the HSA promoter linked to the 1597 bp EcoR1-BstEII fragment of hMusTRD1α1 with the bovine growth hormone polyadenylation signal (bgh). (B) Northern blot of transgene expression (ΔM1) in gastrocnemius muscles of 5 independent lines using a transgene specific probe (top panel). Western blot of ΔhMusTRD1α1 protein expression in transgenic lines (bottom panel). (C) Quantitative analysis of transgene transcript (white) and protein (black) levels in five independent lines, n=4-5 individual progeny. X-axis labels refer to the transgenic line. Data on the Y-axis represent relative densitometric units. (D) Mammogram of a ΔhMusTRD1α1 transgenic mouse showing profound spinal curvature and limb contracture, compared with an age- and sex-matched control on the left. (E) ΔhMusTRD1α1 transgenic mice (right) have poor posture (control on the left) and splayed hindlimbs (F). Disruption of MusTRD function causes growth retardation. F₁ progeny of two independent transgenic lines were weighed immediately after weaning for up to twelve months of age. (G) Growth rates of ΔhMusTRD1α1 male mice from lines 10 (closed squares) and 11 (closed circles) compared with wt littermates (open symbols) during their exponential growth phase (3-7 weeks following birth). (H) Transgenic mice remained growth retarded up to 12 months of age. Data represent the mean weight (g±SD) of 4-14 individual mice. Differences were statistically significant at all time points (0.001<P<0.05). The growth patterns of female transgenic mice were similar to those of male transgenics.

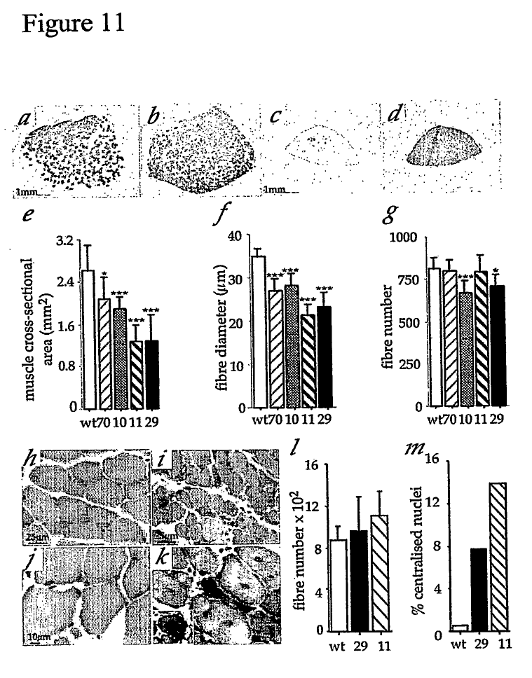

FIG. 11. Histomorphological analysis of ΔhMusTRD1α1 soleus. Immunostaining of wt soleus cross-sections show MHC-I_{slow} positive (A) and MHC-IIA_{fast} positive (B) myofibers (visualised by a brown precipitate). ΔhMusTRD1α1 muscles lack MHC-I_{slow} expression (C) but MHC-IIA_{fast} is expressed in all fibers (D). Quantitative image analyses of soleus muscles from wt mice and 4 independent transgenic lines (70, 10, 11, 29) show ΔhMusTRD1α1 lines exhibit reduced muscle cross-sectional area (E), reduced fiber diameter (F), with slight changes in fiber number (G). Data represent 5-6 individual mice from each line, and 11 wt mice. For E-G, * denotes a significant difference relative to wt with P≦0.05, *** denotes P≦0.01. The calibration bars in A-D represent 1 mm. Degeneration and regeneration in fast myofibers of ΔhMusTRD1α1 mice. H&E staining of a wt (H) and ΔhMusTRD1α1 (I) EDL muscle sections. α-Naphthol acetate esterase assay of macrophage activation (black granules), indicative of fiber degeneration, in ΔhMusTRD1α1 (K) but not in wt muscle (J). Inset in (K) shows a cluster of macrophages infiltrating a myofiber. Fiber number was slightly increased in ΔhMusTRD1α1 EDL muscles (L), consistent with an increase in the number of regenerating (centrally nucleated) fibers (M). Muscles analysed were from 7 week-old mice. The calibration bars in H,I represent 25 μm and in J,K they represent 10 μm.

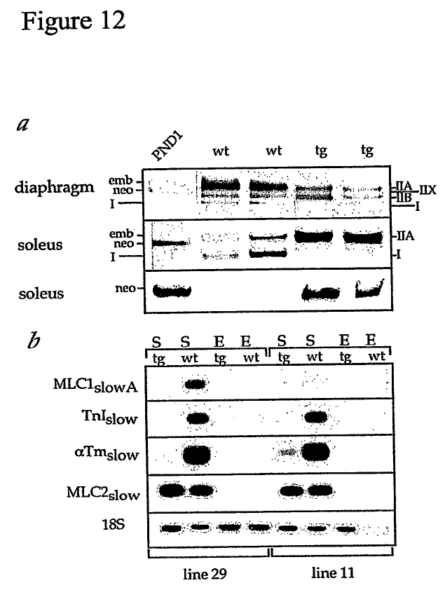

FIG. 12. Fibre-specific gene expression profile of ΔhMusTRD1α1 muscles. (A) Electrophoretic analysis followed by silver staining of protein lysates of diaphragm (top panel), and soleus (middle panel) muscles of 7 week-old ΔhMusTRD1α1 mice from line 29 (tg) and control littermates (wt). The electrophoretic mobility of MHC-I_{slow} (I), MHC-IIA_{fast} (IIA), MHC-IIB_{fast} (IIB), MHC-IIX (IIX), MHC embryonic (emb) and MHC neonatal (neo) are indicated. ΔhMusTRD1α1 muscles exhibit an immature myogenic phenotype as evidenced by the presence of MHCₙₑₒ. Corresponding Western blot of soleus lysates (bottom panel) using an antibody to MHCₙₑₒ and comparison with MHCₙₑₒ expression normally found in mouse muscles at postnatal day I (PND1), confirms expression of MHCₙₑₒ in ΔhMusTRD1α1 muscles. (B) The slow myofiber phenotype is repressed in ΔhMusTRD1α1 mice. Northern blot analysis of total RNA extracted from soleus (S) and EDL (S) muscles of lines 29 and 11 at 7 weeks shows expression of MLC1_{slowA}, Tnl_{slow}, αTm_{slow} and MLC2_{slow} in ΔhMusTRD1α1 (tg) versus wild-type (wt) mice. Samples were loaded relative to 18S rRNA expression.

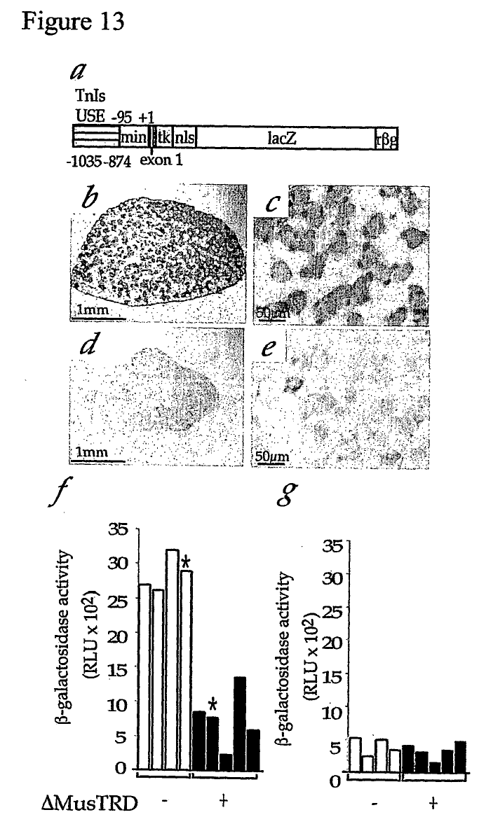

FIG. 13. ΔhMusTRD1α1 represses expression of the Tnl_{slow} upstream enhancer (USE) in transgenic mice. (A) Tnl_{slow}USE-95X1nucZ reporter construct contains the USE (nucleotides −1035 to −875) and minimal promoter (−95 to +1) of the Tnl_{slow} gene, the thymidine kinase 5′ UTR and AUG, and a nuclear localisation signal (nls) upstream of the β-galactosidase gene. rbg denotes rabbit β-globin polyadenylation signal. (B,C) β-galactosidase expression in the soleus muscles of Tnl_{slow}USE-95X1nucZ^{+/+} mice is restricted to MHC-I_{slow} expressing fibers (visualised as a brown precipitate). (D,E) Slow fiber-specific expression is down-regulated in Tnl_{slow}USE-95X1nucZ^{+/+} X ΔhMusTRD1α1^{+/-} muscle. Note the calibration bars in B and D represent 1 mm and those in C and E represent 50 μm. Graphed data represent β-galactosidase activity of protein lysates from soleus (F) and EDL (G) muscles of Tnl_{slow}USE-95X1nucZ^{+/+} mice (open bars) and Tnl_{slow}USE-95X1nucZ^{+/+} X ΔhMusTRD1α1^{+/-} mice (closed bars). Each vertical bar represents β-galactosidase activity in a muscle from an individual mouse. (*) indicate the lines sectioned in panels B-E.

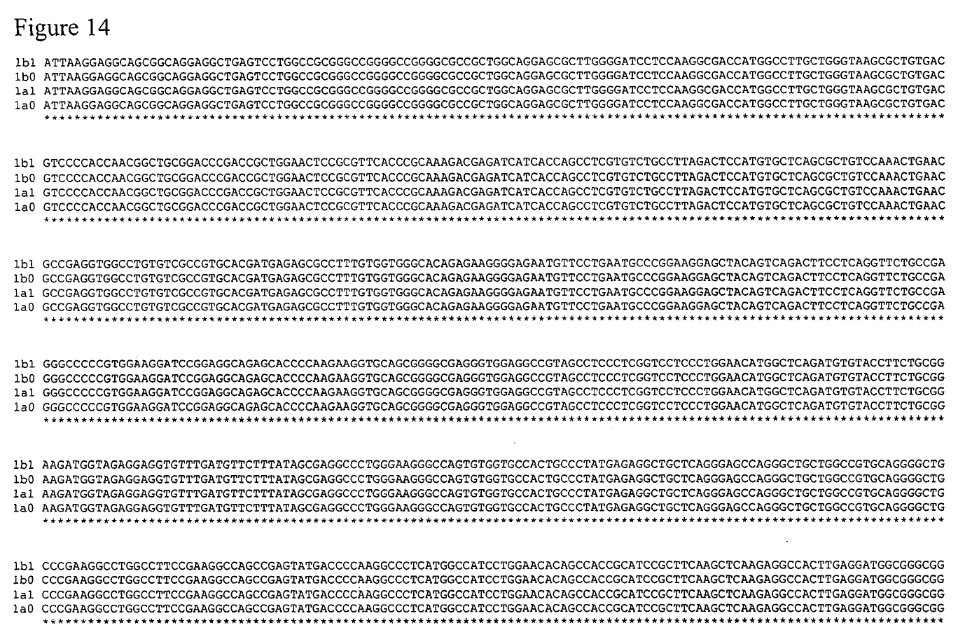

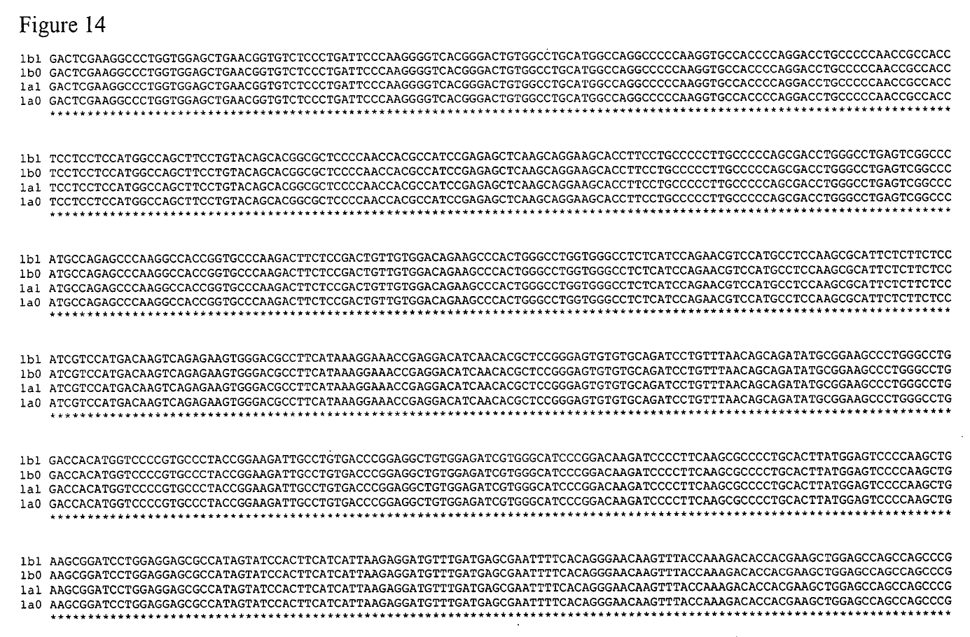

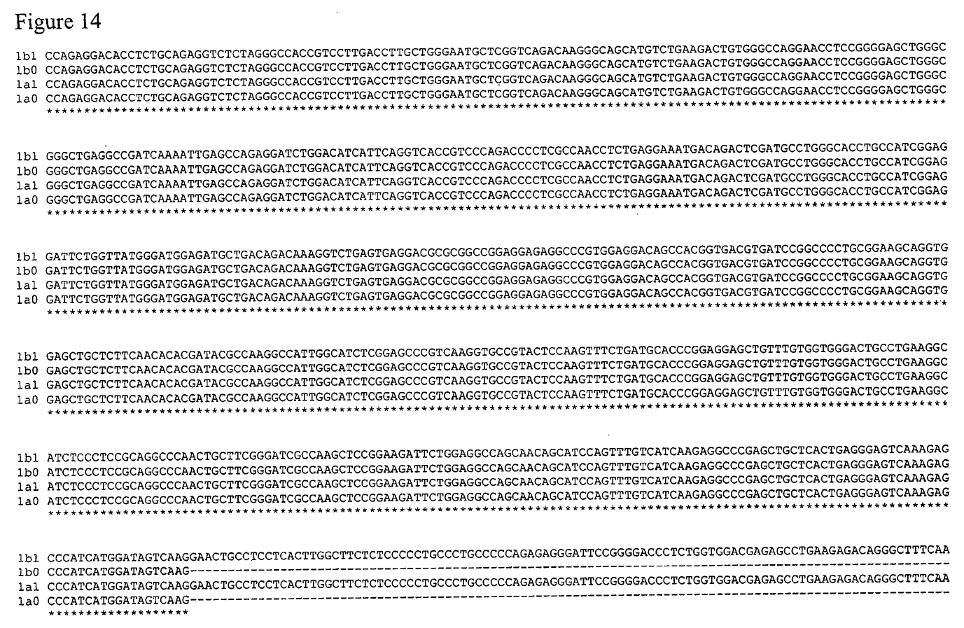

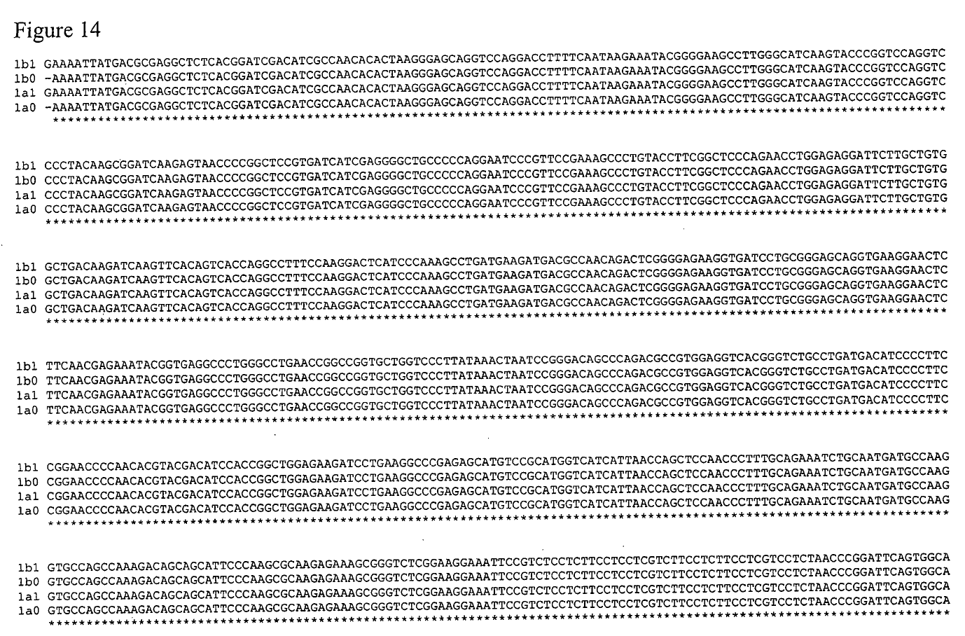

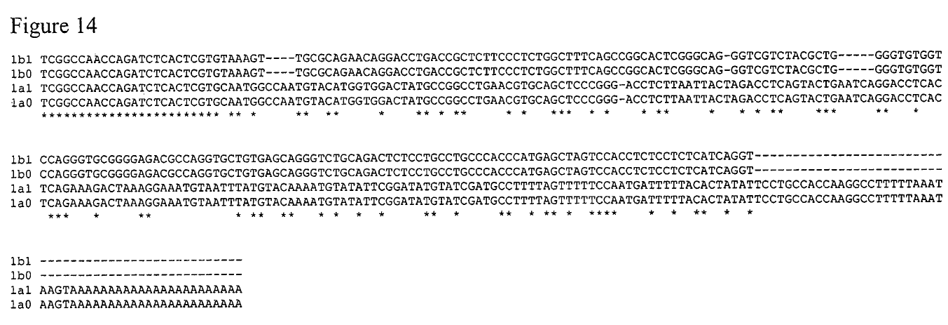

FIG. 14. Alignment of the amino acid sequences of 4 human MusTRD isoforms.

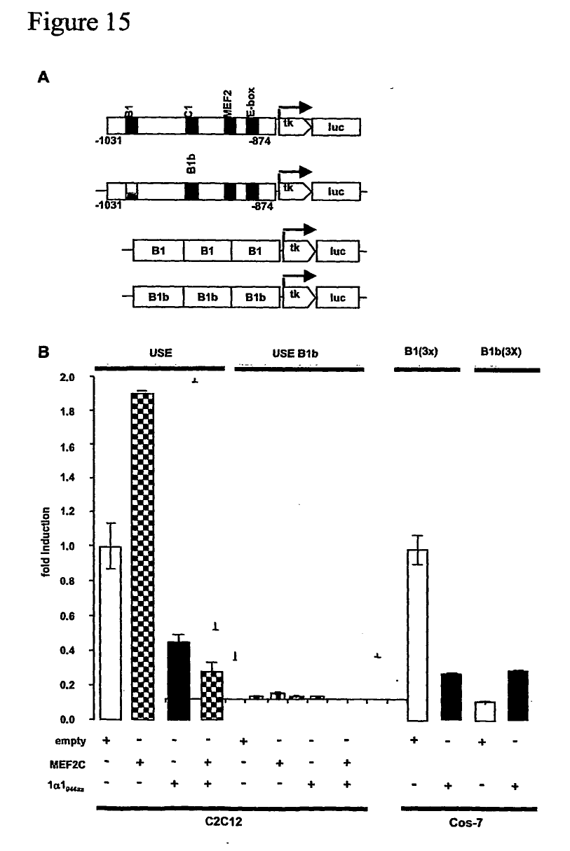

FIG. 15. hMusTRD1α1 represses MEF2C activation through the B1 element in the hTnl_{slow} USE. Transient co-transfections of C2C12 cells were performed using expression constructs for hMusTRD1α1 (1α1₉₄₄ₐₐ) and MEF2C and (A) reporter constructs containing the intact hTnl_{slow} USE (nts −1031 to −874) and mutated USE B1b site linked to luciferase. (B) MEF2C-mediated transactivation and hMusTRD1α1-mediated repression of MEF2C transactivation of the hTnl_{slow} USE in C1C12 cells is expressed as fold induction of basal (empty) expression vector activity which was set at 1. Transient transfections of Cos-7 cells were performed using the expression construct for hMusTRD1α1 (1α1₉₄₄ₐₐ), and (A) reporter constructs containing the trimerized B1 and B1b elements from the hTnl_{slow} USE (nts −977 to −960) linked to luciferase. (B) hMusTRD1α1-mediated repression of B1 transcriptional activity in Cos-7 cells is expressed as fold induction of basal (empty) expression vector activity which was set at 1. Columns represent mean values of triplicates; bars indicate standard error of the mean (SEM).

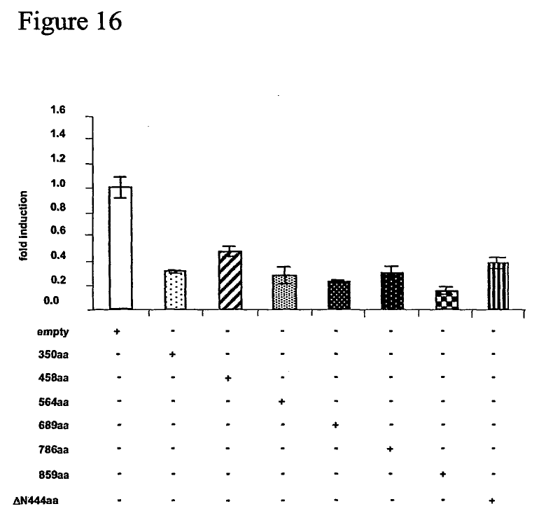

FIG. 16. hMusTRD1α1 can repress in the absence of binding to its cognate binding site. Transient transfections of Cos-7 cells were performed using the series of C-terminally deleted expression constructs for hMusTRD1α1 and the reporter construct containing the trimerized Bi element. The transcriptional activity of each truncated construct is expressed as fold induction of basal (empty) expression vector activity that was set at 1. Columns represent mean values of triplicates; bars indicate SEM.

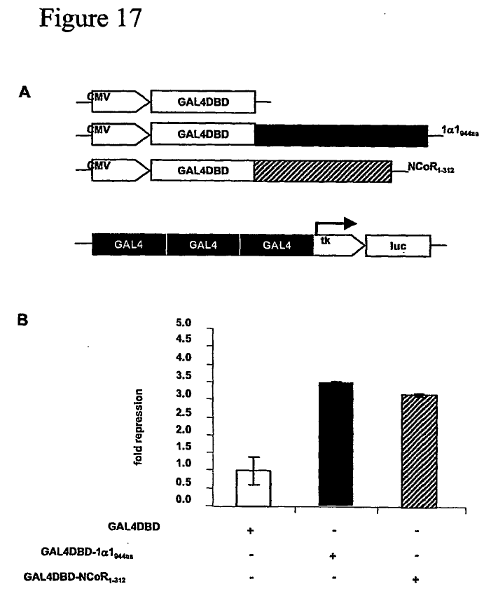

FIG. 17. hMusTRD1α1 and NCoR repress the hTnl_{slow} USE. (A) Mammalian one-hybrid co-transfection assays in Cos-7 cells were performed with the indicated expression constructs: GAL4 DNA binding domain (GAL4DBD), GAL4DBD-hMusTRD1α1₉₄₄ₐₐmyc/his (GAL4DBD-1α1₉₄₄ₐₐ) and GAL4DBD-NCoR₁₋₃₁₂ fusion constructs in addition to a luciferase reporter construct driven by three copies of the GAL4 binding site. (B) Repression is expressed as fold repression of basal GAL4DBD transcriptional activity that was set at 1. Columns represent mean values of triplicates; bars indicate SEM.

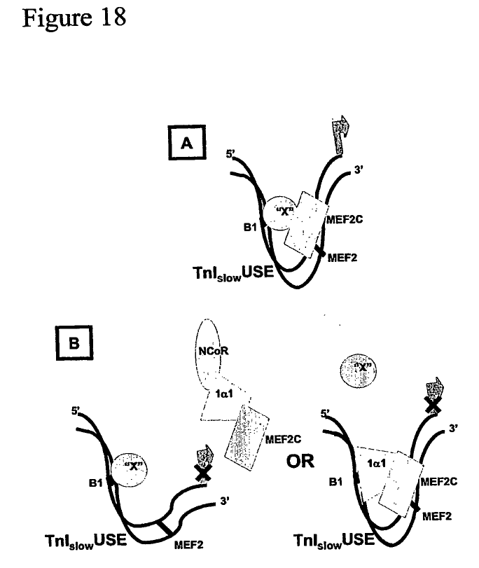

FIG. 18. Mechanisms of hMusTRD1α1 mediated repression of the hTnl_{slow} USE. (A) Transcriptional transactivation through the hTnl_{slow} USE is achieved when the B1 element is occupied by an enhancing factor (X) interacting with MEF2C bound to the 3′MEF2C site. (B) Repression is achieved either by the sequestering of MEF2C by an NCoR:hMusTRD1α1 (1α1) complex or occupation of the B1 element by hMusTRD1α1 that prevents the binding of the enhancing factor.

### EXAMPLES

Introduction

Human MusTRD1α1 (hMusTRD1α1) was originally isolated from a skeletal muscle library (O'Mahoney et al, 1998). hMusTRD1α1 shares homology with the ubiquitous transcription factor TFII-I. We have isolated 11 splice variants from different skeletal muscles which have variably spliced exons in the carboxy terminus. To investigate the role of hMusTRD1α1 and related isoforms in skeletal muscle fiber development, we examined the regulatory potential of the normal hMusTRD1α1 and a 458aa truncated (Δ) peptide of hMusTRD1α1 common to all MusTRD isoforms. We also investigated the mechanism of transcriptional repression by hMusTRD1α1.

Methods

Isolation and Characterisation of cDNA Clones

Plaque forming units (2.4×10⁶) from a mouse skeletal muscle 5′ plus stretch lambda gt11 cDNA library (Clonetech) were screened with a random primer labelled (Giga Prime Labelling Kit; Geneworks) 1.3 kb AatII/PstI fragment of hMusTRD (Acc. No. NM_{—}005685), 28 to 1364 bp downstream of the start codon and containing repeat domains 1 and 2. Hybridisation was carried out in CHURCH (0.5M Na₂HPO₄ pH 7.2, 1% BSA, 7% SDS, 1 mM EDTA pH8) at 50° C. overnight and washed at 60° C. in 0.5×SSC/0.1%SDS with 3 changes over 30 min. Positive plaques were selected and subjected to secondary and tertiary screens employing the same hybridisation conditions. Lambda DNA from positive clones was purified using Qiagen Lambda System Maxi Kit (Qiagen) according to manufacturers instructions. cDNA was isolated from lambda gt11 vector by EcoRI digestion.

Rapid Amplification of cDNA Ends (RACE)

5′ RACE PCR was carried out using a mouse skeletal muscle Marathon-Ready cDNA library (Clontech). PCR was performed using a reverse primer, specific to the cDNA clones (5′-GATCCCACTTCTCTGACTTGTCATG-3′) located downstream of RD2 and the AP1 forward primer (Marathon cDNA Adaptor primer; Clonetech) with Advantage HF PCR polymerase mix (Clonetech) and MasterAmp PCR Optimisation Buffer D (Epicentre Technologies) under the following conditions; 94° C. for 5 s; 72° C. for 4 min for a duration of 5 cycles; 94° C. for 5 s; 70° C. for 4 min for a duration of 5 cycles; 94° C. for 5 s; 68° C. for 4 min for a duration of 30 cycles.

Southern Blotting

Blots were hybridised overnight with a hMusTRD 300 bp BamHl probe (O'Mahoney et al., 1998) or the mouse est clone 555547 (Acc. No. AA111609) (4-103 bp) probe labelled with a random primer labelling kit (Giga Prime Labelling Kit, Geneworks) at 65° C. in CHURCH and washed at 65° C. in 0.5×SSC/0.1%SDS with 3 changes over 30 min.

RNA Isolation from Cells and Adult Tissue

Total RNA was isolated from differentiated myotube C2C12 myotubes according to the protocol of Schmitt et al (1990).

Total RNA was isolated from 20 B6D2 13.5 days post coitus (d.p.c) embryos by Trizol® extraction according to the manufacturer's instructions. Adult vastus lateralis (VL), soleus and extensor digitorium longus (EDL) muscles were isolated from 120 10 week-old female ARCs and total RNA prepared by TriZOI™ extraction. Poly-A RNA was then purified with Dynabeads (DYNAL) according to the manufacturers instructions.

In Example 3, total RNA was extracted by the Trizol® method (Invitrogen) from Cos-7 cells, undifferentiated C2C12 myoblasts and C2C12 cultures containing myotubes that had been allowed to differentiate for 3 days.

RT-PCR

Poly-A RNA (1 μg) was primed with 40 pmole Oligo d(T)₁₀ decanucleotides (Roche) and reverse transcribed with Superscript II reverse transcriptase (Life Technologies) according to manufacturers instructions. PCR was carried out using 10% of the RT reaction as template. Amplification of the entire coding sequence was performed under the following conditions; 95° C. for 3 min, 95° C. for 30 s, 55° C. for 30 s, 72° C. for 3 min for 35 cycles with a forward primer designed from the 5′ RACE PCR product upstream of the ATG (5′-CAACCAGA GGCGACTGGATC-3′) and reverse primers specific to each cDNA clone downstream of the stop codon and upstream of the poly-A tail (5′-GGAGGTTGA GTTTCGTCACGTGA-3′ and 5′-TGGCGGCAGGAATATAGTG-3′), using Taq DNA Polymerase (Roche). PCR products were analysed on 1% agarose gel, purified using Qiaquick gel extraction kit (Qiagen) and cloned into pGEM-T Easy vector. The same reverse primers, in combination with two forward primers designed against exon 19 or the coding sequence flanking exon 19 (3′ region of exon 18 and the 5′ region of exon 20) (5′-GACCGTCTTGTGGACGAGACC-3′ and 5′-CTGGACACTCAAGAAAATTACAAC-3′ respectively) were to amplify the 3′ region only, under the following conditions; 95° C. for 3 min, followed by 95° C. for 30 s, 55° C. for 30 s, 72° C. for 1 min for 35 cycles.

In Example 3, first-strand cDNA was synthesised using 2 μg of total RNA with Impromptu® reverse transcriptase (Promega) according to the manufacturer's instructions. The primers MusTRDF (nts 234 to 257; 5′-GAGCTACAGTCAGACTTCCTCAG-3′) and MusTRDR (nts 986 to 1009; 5′-TCTCTGACTTGTCATGGACGATG-3′) were designed to regions of the open reading frame with complete sequence conservation between mouse and human. The PCR amplification containing these primers used 5% of the first-strand cDNA as template, Masteramp Buffer D (Epicentre Technologies) and the cycling parameters: 95° C. 3 min followed by 35 cycles of 95° C. for 30 sec, 60° C. for 1 min, 72° C. for 1 min.

Isoform Screening

RT-PCR reactions of total RNA from differentiated C2C12 myotobes, 13.5 dpc embryos, adult quadriceps, soleus, and EDL muscles were separated on 1.5% agarose gel and the resulting bands purified using Qiaquick gel extraction kit (Qiagen) and cloned into pGEM-T Easy vector. No less than 12 positive clones were amplified and 100 ng of purified plasmid then used as a template for a PCR reaction with forward primers specific for exon 19, the coding sequence flanking exon 19, exon 23 and the coding sequence flanking exon 23 (5′-ACCAGACC AAGGAGACTGCAACAG-3′ and 5′-CAAGGACTTATCCCAAAGCCTGAT-3′, respectively) in combination with two reverse primers designed against exon 30 and exon 31 under the following conditions; 95° C. for 30 s, 55° C. for 30 s, 72° C. for 1 min for 35 cycles. PCR products were then analysed on 1.5% agarose gel.

MusTRD antibody

Sheep anti-MusTRD serum was raised against the first 20 amino acids of hMusTRD1α1 (Mimotopes, Clayton, Victoria). Hyperimmune serum was affinity purified using biocytin-tagged N-terminal peptides coupled to M-280 streptavidin Dynabeads (Dynal®, Cariton, Victoria). Immobilised MusTRD antibody was eluted with 3 M MgCl₂, pH 7.2. Antibody preparations were dialysed in a buffer containing 25 mM Tris-HCl, pH 7.2, 0.15 M NaCl and 0.1% BSA (TBS-BSA) using Slide-A-Lyzer® Mini Dialysis Units (10,000 MWCO; Pierce, Rockford, Ill.). Purified antibody was stored in TBS-BSA with 0.1% Tween-20 at 4° C.

Plasmid constructs

The 157 bp USE element (nts −1031 to −874) from the human Tnl_{slow} gene and a mutated B1b-containing version (O'Mahoney et al., 1998) were linked to the thymidine kinase (tk) minimal promoter (nts +81 to +52) and the luciferase reporter gene, generating the pTnl_{slow}USEB1tkluc and pTnl_{slow}USEB1btkluc reporter constructs (FIG. 1). The P(B1)₃ₓtkluc and p(B1b)₃ₓtkluc reporter constructs were produced by joining three tandem copies of the B1 element (nts −977 to −960; 5′-AGCCACAGG ATTAACATA-3′) and three tandem copies of the mutated B1b version (5′-AGCCACAGGATatcCATA-3′) to the tk minimal promoter and the luciferase reporter gene (FIG. 1). The pcDNAMEF2C expression construct containing the mouse MEF2C cDNA driven by a CMV promoter was a kind gift from Dr. Richard Harvey.

Five hMusTRD1α1 cDNAs with progressive truncations of the 3′ ends were amplified by PCR using hMusTRD1α1 (accession no. AF118270) as a template.

The nucleotide sequences used to amplify the five hMusTRD1α1 C-terminal truncation mutants are shown below. Five reverse primers,

859R:
5′-AGCGGATCCTGATGACCATGCGGAC-3′;
786R:
5′-AGCGGATCCGGATCACCTTCTCCCC-3′;
689R:
5′-AGCGGATCCGTGTGTTGGCGATGTC-3′;
564R:
5′-AGCGGATCCGGGGCCGGATCACGTC-3′;
and
350R:
5′-AGCGGATCCGCGTGTTG ATGTCCTC-3′

were designed against specific amino acid sites within hMusTRD1α1, resulting in truncations before each repeat domain or putative functional region. The forward primer, F1: 5′-GGTCGAATTCA TGGCCTTGCTGGGTAA-3′ was engineered to contain a 5′ EcoRI site (underlined) to facilitate cloning into the pcDNA3.1myc/his vector (Invitrogen). This strategy allowed the production of five mammalian expression plasmids encoding epitope-tagged MusTRD polypeptides of 350aa, 458aa, 564aa, 689aa, and 786aa together with a full-length 944aa construct (not including tag). A seventh plasmid with an internal deletion of amino acids 100-544 was created by AoCI/BstEII digestion and re-ligation of the full-length pcDNA3.1hMusTRD1α1₁₉₄₄ₐₐmyc/his, thereby producing pcDNA3.1 h MusTRD1α1ΔN₄₄₄myc/his.

The pCMXNCoR expression construct containing the entire mouse NCoR cDNA driven by a CMV promoter was a kind gift from Dr. Thorsten Heinzel. Plasmids pCMVGAL4hMusTRD1α1₉₄₄ₐₐ and pGAL4NCoR₁₋₃₁₂ were constructed by fusion of the yeast GAL4 DBD (aa 1-147) with the cDNA of hMusTRD1α1₉₄₄ₐₐ or a fragment of the NcoR cDNA that encodes the amino-terminal 312 aa containing the repression domains (Hörlein et al., 1995). In mammalian one-hybrid assays, the luciferase reporter gene was driven by three tandem copies of the GAL4 binding site fused to the tk promoter to generate p(GAL4)₃ₓtkluc (Umesono et al., 1991; Horlein et al., 1995). pGST-NcoR₁₆₄₉₋₂₄₅₃ and pGST-MusTRD1α1 also used.

Mammalian Expression Constructs.

The cDNAs for hMusTRD1α1₄₅₈ₐₐ and hMusTRD1α1₉₄₄ₐₐ, in addition to the five C-terminally deleted human MusTRD1α1 series ranging in size from 350aa, 564aa, 689aa, 786aa and 859aa, were subcloned into the CMV promoter driven pcDNA3.1 myc/his expression vector (Invitrogen, Sydney, Australia) for production of in vitro translated proteins and for use in transfection assays.

In vitro Protein Translation

In vitro translated hMusTRD1α1 350aa; 458aa; 564aa; 689aa; 786aa; 859aa and 944aa proteins were generated from the respective expression plasmids using the TNT® rabbit reticulocyte system as recommended by the supplier (Promega). [³⁵S]-labelled in vitro translated proteins were routinely checked for translation efficiency by electrophoresis through a 10% SDS-polyacrylamide gel, subsequent gel drying, exposure to a Molecular Dynamics phosphorimaging screen and quantification with the use of a Molecular Dynamics Storm 860 reader (Palo Alto, USA) using ImageQuant software (Molecular Dynamics; Palo Alto, USA).

Gel Shift Assays

In vitro translated hMusTRD1α1 350aa; 458aa; 564aa; 689aa; 786aa; 859aa and 944aa were incubated at room temperature in a total volume of 40 μl of binding buffer (10 mM Hepes (pH 7.9); 1 mM dithiothreitol, 0.2 μg/μl poly (dl-dC) and 10% glycerol). In antibody gel shift assays, 5 μl of an α-hMusTRD1α1₁₋₂₀ₐₐ antibody was pre-incubated with hMusTRD1α1 proteins for 20 min at room temperature prior to addition of approximately 30,000 cpm of ³²P-labelled wild-type enhancer (B1)₃ₓ element or the mutated (B1b)₃ₓ element. This reaction was then incubated for a further 20 min at room temperature. DNA-protein complexes were resolved through a 4% (w/v) non-denaturing polyacrylamide gel in 0.5×TBE (45 mM Tris, 45 mM boric acid, 1 mM EDTA; pH 8.3) and visualised by autoradiography on X-ray film (Kodak BioMax; Rochester, N.Y., USA). Experiments were performed at least three times and a representative result is shown.

Plasmid and Transgene Constructs

The EcoR1 cDNA fragment isolated from pGAD10-hMusTRD1α1 was subcloned into pCI-neo (Promega, Madison, Wis.) and pAS2-1 (CLONTECH; Palo Alto, Calif.) for expression of full-length hMusTRD1α1 in mammalian and yeast cells, respectively. The EcoR1-BstEII (1597bp) fragment was subcloned into EcoRI- Smal sites of pCI-neo for expression of ΔhMusTRD1α1 in mammalian cells. The Tnl_{slow}USE (nucleotides −1031 to −874) luciferase reporter constructs pTK81Luc, pTnl_{slow}USE/TKLuc, pTnl_{slow}ΔB1 USE/TKLuc have been described previously (O'Mahoney et al. 1998). The EcoR1- Pme1 fragment frompcDNA3.1-2000HSA:ΔhMusTRD1α1 was subcloned into EcoR1-Sma1 of pAS2-1 and pACTII (CLONTECH) for analysis in yeast cells.

To generate the hMusTRD1α1 transgenes, pcDNA3.1-2000HSA: hMusTRD1α1 and pcDNA3.1-2000HSA:ΔhMusTRD1α1, first the CMV promoter of pcDNA3.1+ (CLONTECH) was replaced with the human skeletal actin promoter (HSA 2.2 kb Hind III fragment; Brennan and Hardeman, 1993). The hMusTRD1α1 EcoRI and EcoRI-BstEII (blunted) cDNA fragments were cloned into the EcoR1 and EcoR1- EcoRV sites to generate pcDNA3.1-2000HSA:wt-hMusTRD1α1 and pcDNA3.1-2000HSA:ΔhMusTRD1α1, respectively. The transgenic construct Tnl_{slow}USE-95X1nucZ has been described previously (Corin et al. 1995).

Cell Culture and transfection

For the USE/USE-B1b heterologous reporter assays, C2C12 cells were seeded into 24-well plates (5×10⁴ cells/well) and grown overnight in low glucose DMEM (Invitrogen) with 20% FBS and 0.5% chick embryo extract at 37° C. in an atmosphere of 10% CO₂. Transfections were performed using Lipofectamine 2000™ (Invitrogen) according to the manufacturer's instructions with 250 ng of the pTnl_{slow}USEB1tkluc or pTnl_{slow}USEB1btkluc reporter constructs in combination with 100 ng quantities of the pcDNA3.1hMusTRD1α1-truncation series, pMEF2C or combinations of the above. The cells were incubated in the transfection medium for 5 hrs before replacement with differentiation media containing DMEM with 2% horse serum (HS) and the resulting cultures, containing differentiated myotubes, were harvested. 36 hrs later.

For the B1/B1b heterologous reporter assays and the mammalian one-hybrid assays, 1×10⁵ Cos-7 cells were seeded into each well of a 12-well tissue culture plate and grown overnight in DMEM with 10% FBS. Cos-7 cells were transfected with 500 ng of the p(B1)₃ₓtkluc, p(B1b)₃ₓtkluc or p(GAL4)₃ₓtkluc reporter constructs in combination with 200 ng quantities of pcDNA3.1hMusTRD1α1₉₄₄ₐₐmyc/his, pCMVGAL4hMusTRD1α1₉₄₄ₐₐ, pCMVGAL4NCoR₁₋₃₁₂ or the pGAL4DBD expression plasmids using 1.5 μg of Fugene6™ (Roche) in 1 ml of unsupplemented DMEM for 5 hrs. The transfection media was replaced with DMEM containing 10% FBS and cells were harvested 40 hrs post-transfection.

For immunoprecipitation experiments and antibody detection of MusTRD protein on Western blots, approximately 1×10⁷ Cos-7 cells were transfected with 20 μl of pCMXNCoR, pcDNA3.1hMusTRD1α1₃₅₀myc/his or pcDNA3.1hMusTRD1α1₉₄₄ₐₐmyc/his using Lipofectamine Plush™ (Invitrogen) according to the manufacturer's protocol.

COS-7 Cell Transfections

COS-7 cells were grown in DMEM supplemented with 10% foetal calf serum and seeded at 4×10⁶ cells/150 mm dish for transfection. Following overnight growth, 10 μg of plasmid DNA pCI-neo, pCI-neo:ΔhMusTRD1α1 or pCI-neo:hMusTRD1α1 was coupled to Fugene™ and applied to the cell monolayer according to the manufacturer's instructions (Roche Diagnostics). After 48 hours, cells were harvested, lysed in RIPA buffer (150 mM NaCl, 50 mM Tris-HCl pH 8, 0.5% sodium deoxycholate, 0.1% SDS, 1% NP-40), and used as positive controls in Western blotting.

Plasmid Constructs for C2C12 Transfections

The cDNAs for human hMusTRD1α1₉₄₄ₐₐ (FL) and hMusTRD1α1₄₅₈ₐₐ(TR) and mouse isoforms MusTRD3α7, MusTRD1β1 and MusTRD3β7 were subcloned into Age I site of the CMV promoter driven pcDNA3.1 myc/his expression vector (Invitrogen, Sydney, Australia). Reporter plasmid USE-TK-Luciferase, contains hTnIs upstream enhancer (USE; 157 bp from −1031 to −875). (−3500)βMHC-pGL3 and (−2554 to +13) IIβMHC-pGL3 reporters are described in di Maso et al., 2000; Wright et al., 2001. The luciferase reporter construct −800MLC2s is described in Esser et al., 1999.

C2C12 Transient Transfections

Mouse myoblast (C2C12) cells were grown as monolayers in Dulbeccos modified Eagle medium (DMEM) supplemented with 20% fetal calf serum, 0.5% chicken embryo extract (all from Gibco BRL) at 37° C. in 10% CO₂. For transfection, C2C12 cells were plated at 30-50% confluence in 24-well plates over-night and the next day cells were cotransfected with 250 ng of reporter plasmids and 100 ng of pcDNA3.1 or pcDNA3.1 containing cDNA for human or mouse isoforms expression plasmids by Lipofectamin® following the protocol of the manufacturer (Gibco, BRL). After six hours incubation at 37° C. in 10% CO₂, the medium containing the DNA-Lipofectamin® mix was removed and fresh medium supplemented with 2% horse serum (differentiation medium) was added. After incubation for 36 h in differentiation medium, the cells were lysed and luciferase activity was determined in a TopCount Microplate Scintilation & Luminescence counter (Packard). Each experimental condition was measured in triplicates and the values given represent the mean±standard deviation from three experimental occasions.

MusTRD Western Blot

Adult mouse gastrocnemius muscles were crushed and dissolved in RIPA buffer. Tissues were homogenised and lysed on ice for 1 h, then centrifuged at 14,000 rpm for 1 h at 4° C. Soluble muscle protein (200-500 μg) and COS-7 cell lysates (50 μg) were analysed on 10% SDS-PAGE gels. Gels were electroblotted onto nitrocellulose membranes and blocked for 16 h at 4° C. with TBS containing 0.1% Tween-20 (TBST), 5% skim milk powder and 1% BSA. Membranes were probed with purified sheep anti-MusTRD antibody (1:100) in TBST containing 1% BSA for 2 h at 22° C., followed by donkey anti-sheep peroxidase-conjugate (1/20,000; Sigma, Castle Hill, NSW) for 1 h at 22° C. Immunoreactive bands were detected by chemiluminescence (Lumi-Light^{PLUS}; Roche Diagnostics, Castle Hill, NSW).

Confocal Microscopy

Six μm sections of mouse soleus muscle were fixed in 2% buffered paraformaldehyde for 10 min at 4° C., washed in ice cold TBS containing 0.2% Tween-20, then blocked with 10% donkey serum in TBST for 30 min at 22° C. Sections were exposed to purified sheep anti-MusTRD antibody (1:5-1:10) in TBST containing 10% donkey serum overnight at 4° C., followed by donkey anti-sheep IgG conjugated to FITC (1:50 dilution) for 1 h at 22° C. (Sigma). Slides were washed and relabelled with a mixture of mouse anti-dystrophin (DYS2, Novocastra Laboratories, Benton Lane, Newcastle) and mouse antibody for type I myosin heavy chain (BAF8; Borrione et al. 1988) overnight at 4° C. Goat anti-mouse rhodamine red-X conjugated antibody was applied for 90 min at 22° C. (Jackson ImmunoResearch, West Grove, Pa.). Muscle nuclei were stained with propidium iodide and the slides mounted in 2.5% DABCO in 80% glycerol. Sections were visualised with a Leica confocal laser scanning microscope.

Electromobility Shift Assay

Mouse EDL and soleus muscle were used to prepare nuclear extracts by the method described previously (O'Mahoney et al. 1998). Nuclear proteins were subjected to electromobility shift analysis with oligonucleotides USE-B1 (5′-AGCCACAGGATTAACATA-3′) and USE-B1b (5′-AGCCACAGGATATCC ATA-3′; O'Mahoney et al. 1998).

hMusTRD1α1 350aa, 458aa, 500aa, 564aa, 689aa, 786aa and 944aa proteins and the MEF2C protein were produced by in vitro coupled transcription-translation reactions using TNT® rabbit reticulocyte lysate (Promega) in the presence of [³⁵S] methionine (Amersham Pharmacia). Protein production efficiency was tested by 10% SDS polyacrylamide electrophoresis (SDS-PAGE) of the reactions, followed by exposure of the dried gels to phosphorimaging screens and quantification using a Molecular Dynamics Storm 860 analyser and ImageQuant software (Molecular Dynamics).

Oligonucleotides (B1₃ₓ, B1b₃ₓ, 3′MEF2 [nts −908 to −891 from the Tnl_{slow} USE] or mut3′MEF2 were labeled using Klenow to fill 3′ overhangs in the presence of ³²PαdCTP. Quantities of labelled oligonucleotide corresponding to 30,000 cpm were allowed to bind to the in vitro translated hMusTRD1α1 or MEF2C polypeptides in 40 μl of binding buffer (10 mM Hepes (pH 7.9), 1 mM dithiothreitol, 0.2 μg/μl poly (dl-dC), 10% glycerol) for 20 min at RT. In assays involving antibodies, the sheep anti-hMusTRD1α1₁₋₂₀ₐₐ polyclonal antibody was pre-incubated with hMusTRD1α1 polypeptides for 20 min at RT prior to addition of the oligonucleotides and in competition shift assays, 4 μl, 8 μl and 12 μl of in vitro translated hMusTRD1α1₉₄₄ₐₐ was pre-incubated with MEF2C protein for 20 min at RT. DNA-protein complexes were electrophoresed through a 4% (w/v) non-denaturing polyacrylamide gel in 0.5×TBE (45 mM Tris, 45 mM boric acid, 1 mM EDTA, pH 8.3) and visualised by autoradiography (Kodak BioMax; Rochester, N.Y., USA). Experiments were performed at least three times and a representative result is shown.

Protein Analysis

NCoR-containing complexes were analysed by harvesting the Cos-7 cells in 1×NP40 lysis buffer (20 mM Tris, pH 7.5, 1 mM EDTA, 1 mM EGTA, 0.1 mM PMSF, 150 mM NaCl, 1% Nonidet P-40 containing Completes™ [Roche] protease inhibitor cocktail) 24 hrs after plasmid transfection. The lysates were mixed with an anti-NCoR Rb88 polyclonal antibody directed against the C-terminal 2239-2453aa region (a kind gift from Dr. Thorsten Heinzel, Georg Speyer Haus, Frankfurt am Main, Germany) and protein A/G agarose (Santa Cruz) and incubated at 4° C. for 30 mins. Complexes bound to the agarose beads were washed in 1×NP40 lysis buffer, then boiled in SDS loading buffer and subjected to 10% SDS-PAGE before transfer onto PVDF HybondP membrane (Amersham Pharmacia). The filter was incubated with the 9E10 anti-c-myc monoclonal antibody (Santa Cruz) and binding of the secondary antibody was detected using ECL Super Signal™ (Pierce).

The ectopically expressed hMusTRD1α1₉₄₄ₐₐ protein was visualised by harvesting the Cos-7 cells in 1×NP40 lysis buffer 24 hrs after transfection. Lysates were separated through a 10% SDS-polyacrylamide gel and analysed on a Western transfer blot using the sheep anti-hMusTRD1 α1₁₋₂₀ₐₐ polyclonal antibody.

Luciferase assays were performed by lysis of the cultured cells in the reporter gene lysis buffer and the constant light signal luciferase reporter gene assay was conducted according to the supplier's protocol (Promega) using a Top Count Microplate Scintilation & Luminescence counter (Packard). Luciferase activities were normalised to protein concentration and expressed as ratios relative to the activity in cells transfected with empty vector. Experiments were performed at least three times and a representative result is shown.

GST Pull-down Assays

Expression of GST-NCoR₁₆₄₉₋₂₄₅₃, GST-MusTRD1α1₉₄₄ and GST alone in the BL21(DE3)pLysS Gold E. Coli strain was induced with isopropyl-b-D-thio-galactopyranoside (IPTG, 1.25 mM) for 3 hrs at 30° C. Protein production was checked by Coomassie brilliant blue staining of SDS-PAGE gels. Glutathione-sepharose slurries were pre-blocked in PPI buffer containing bovine serum albumin (1 μg/μl) prior to use. GST pull-down assays were performed by co-incubation of GST or GST fusion proteins with in vitro translated [³⁵S]-labelled hMusTRD1α1₃₅₀ₐₐ or MEF2C for 40 min at RT in PPI buffer (20 mM Hepes, pH 7.9; 200 mM KCl; 1 mM EDTA, 4 mM MgCl₂, 1 mM dithiothreitol; 0.1% NP40 and 10% glycerol). The protein-bound glutathione sepharose slurry was washed several times in PPI, boiled in SDS loading buffer and subjected to 10% SDS-PAGE. The gels were dried, exposed to a phosphorimaging screen and the bands quantified using a Molecular Dynamics Storm 860 reader and ImageQuant software (Molecular Dynamics).

Northern Blot Analysis

A cDNA clone was isolated from a mouse quadriceps muscle cDNA library (CLONTECH) by hybridisation to a hMusTRD1α1 cDNA probe (5′ 300 bp BamH1 fragment). Sequences corresponding to the RD1-RD2 intervening region of human hMusTRD1α1 (nucleotides 675-1066) were amplified by PCR to generate a probe that was labelled via random priming using the Giga-Prime™ DNA Labelling kit (Geneworks, Thebarton, SA), according to the manufacturer's instructions. A mouse embryonic poly-A⁺ RNA blot (CLONTECH) was pre-hybridised with Ultrahyb solution (Ambion, Inc., Austin, Tex.), hybridised with the probe at 42° C., and washed according to the manufacturer's instructions (Ultrahyb protocol; Ambion). Poly-A⁺ RNA loading of the CLONTECH blot was checked by hybridisation with a β-actin probe (CLONTECH) using Ultrahyb conditions (Ambion). For analysis of contractile protein gene isoform expression, the soleus and EDL muscles of four transgenic progeny were pooled and total RNA isolated and processed by Trizol reagent (Sigma) or by acid guanidinium thiocyanate-phenol-chloroform extraction. cDNA probes for MLC1_{slowA}, Tnl_{slow}, αTm_{slow}, MLC2_{slow} have been described previously (Sutherland et al. 1991; Zhu et al. 1995).

Yeast Two-hybrid Assay

The dual reporter YM4271 yeast strains containing the USE-B1 and USE-B1b tandem repeats have been described previously (O'Mahoney et al. 1998). A two-hybrid mating assay was used to examine the transactivation and dimerisation functions of hMusTRD1α1. The yeast reporter strain Y190 (CLONTECH) was transformed with 1 μg of pAS2-1, pAS2-1:hMusTRD1α1, or pAS2-1:ΔhMusTRD1α1 using an alkali cation method (BIO 101, Inc., La Jolla, Calif.) and mated with Y187 cells carrying either pACTII, pGAD10:hMusTRD1α1 or, pACTII:ΔhMusTRD1α1. Transformants were selected on complete synthetic media plates lacking tryptophan and leucine (BIO 101). Diploid cells were grown to saturation and used to inoculate test cultures (OD₆₀₀=0.1) that were grown for a further 24 h at 30° C. Cells were harvested in 100 μl Breaking Buffer (100 mM Tris-HCl pH 8, 20% glycerol, 5 mM PMSF, 2 mM DTT) and lysed by vortexing with glass beads. Protein concentration was determined by the Bradford method (Bio-Rad Laboratories, Sydney, NSW). Protein extracts (20 μg) were assayed for β-galactosidase activity with a chemiluminescence detection kit (CLONTECH) and a Turner Designs 20/20 luminometer (Sunnyvale, Calif.).

Generation and Analysis of hMusTRD1α1 and ΔhMusTRD1α1 Transgenic Mice

Transgenic mice were generated and genotyped by standard methods (Brennan and Hardeman, 1993). Five lines of −2000HSA:ΔhMusTRD1α1 transgenics and 7 lines of −2000HSA:hMusTRD1α1 were genotyped by Southern blotting and determined to carry one transgenic locus each. ΔhMusTRD1α1 protein expression levels were measured by Western blotting of gastrocnemius muscle extracts (100 μg). Western blots were scanned with a Computing Densitometer and analysed using ImageQuant 5.0 (Molecular Dynamics, Sunnyvale, Calif.). ΔhMusTRD1α1 immunoreactive bands were normalised for total protein. Total RNA was isolated from the contralateral gastrocnemius muscle and processed for Northern blot analysis. Samples (3 μg) were blotted with a human hMusTRD1α1 cDNA probe (5′ 300 bp BamH1 fragment) and re-blotted with an 18S probe, according to the protocol described in Sutherland et al. (1991). Northern blots were scanned and analysed as for Western blots. ΔhMusTRD1α1 mRNA expression was normalised to 18S expression. One wild-type male mouse (8 wk old) and one ΔhMusTRD1α1 male mouse with kyphosis were anaesthetised and X-rayed.

Growth Study

The rate of growth of two independent −2000HSA:ΔhMusTRD1α1 transgenic lines (10 and 11) was monitored by weighing the mice as follows: i) at weaning or 21 days postnatal, ii) every second day for two weeks, iii) every week up to 7 weeks of age, iv) every fortnight up to twelve months of age.

Histochemistry and MHC Immunochemistry

At 7 weeks of age, 4-6 wt and −2000HSA:ΔhMusTRD1α1 mice were sacrificed and the soleus and EDL muscles removed, mounted in freezing medium and stored under liquid nitrogen. Haematoxylin and eosin (H&E) staining was performed on 20 μm muscle sections to determine fiber number. Immunohistochemical staining for MHC was performed as described previously (Schiaffino et al. 1989). 20 μm muscle sections were incubated with supernatant from hybridomas secreting antibodies against MHC type I (BAF8; Borrione et al. 1988), type IIA and IIB (SC-71 and BF-F3; Schiaffino et al. 1989) all purchased from the German Collection of Microorganisms and Cell Cultures, and visualised with immunoperoxidase detection. Macrophage infiltration in EDL muscles was detected by an α-naphthol acetate esterase activity assay on 8 μm sections according to the manufacturer's instructions (Sigma Diagnostics, Castle Hill, NSW; Bhatia et al. 1994). Macrophage-specific enzyme activity was validated by blocking with 1 mM sodium fluoride.

Homozygote Tnl_{slow}USE-95X1nucZ transgenics (Corin et al. 1995) were intercrossed with F₁ generation −2000HSA:ΔhMusTRD1α1 transgenics. At 2 and 7 weeks of age, mice were sacrificed, soleus and EDL muscles collected for detection of β-galactosidase activity and histological analysis as described previously (O'Mahoney et al. 1998). At 8 weeks of age, crural muscle blocks were collected and frozen from wt, −2000HSA:hMusTRD1α1, and −2000HSA:ΔhMusTRD1α1 mice, and 20 μm sections incubated with BAF8 supernatant. Muscle histological images were visualised with an Olympus B×50 microscope, captured using a SPOT-Advanced digital camera (Diagnostic Instruments, Inc. Sterling Heights, Mich.) and analysed using Image-Pro® Plus (Version 4.0; Media Cybernetics, Silver-Spring, Md.).

MHC Gel Electrophoresis

Contralateral soleus, EDL muscles and diaphragm were snap frozen, crushed, dissolved in 4 volumes of extraction buffer (0.3 M NaCl, 150 mM NaPO₄ buffer pH 6.5, 100 mM sodium pyrophosphate, 1 mM MgCl₂, 10 mM EDTA, 1.4 mM β-mercaptoethanol), processed, and separated by PAGE as described previously (Butler-Browne et al. 1984). Protein gels were visualised by silver-staining (Bio-rad) or subjected to Western blotting with antibodies raised to the neonatal isoform of MHC (NCL-MHCn; Novocastra), as described previously.

### Example 1

### Isolation and Characterisation of mMusTRD Isoforms

A cDNA library screen resulted in the identification of 7 cDNA clones that were isolated from the lambda vector and cloned into pGem7Zf(+) (Promega) for sequencing. Sequence analysis revealed the absence of the 5′ region of the open reading frame containing the start codon in each clone. This was due to the presence of an internal EcoRI recognition site located within RD1 (repeat domain 1) that resulted in a truncation of the 5′ region when cDNA was isolated from lambda gt11 vector by EcoRI digestion. To obtain this 5′ region, 5′ RACE PCR was carried out. PCR products were southern blotted and hybridised with a hMusTRD 300 bp BamHI probe (O'Mahoney et al., 1998) or the mouse est clone 555547 (Acc. No. AA111609) 4-103 bp. Two products hybridising to both probes were cloned into pGEM-T Easy vector (Promega) and sequenced, one of which was found to overlap with 100% identity to each of the cDNA clones isolated from the original mouse skeletal muscle library screen. BLAST search revealed that the PCR product was completely homologous with mouse est clone 555547 (results not shown).

RT-PCR using a forward primer (5′-CAACCAGAGGCGACTGGATC-3′) based on the 5′ RACE PCR product and reverse primer designed against either exon 30 or 31, downstream of the stop codon and upstream of the poly-A tail, on cDNA derived from soleus, EDL or 13.5 d.p.c. embryonic total RNA produced the open reading frames of 8 isoforms. Analysis of the 5′ regions between 1 and 1966 bp revealed complete homology between all 8 isoforms. Sequence analysis of these isoforms and a database search indicate the presence of one gene coding for these isoforms, the GTF2IRD1 gene on chromosome 5, which contains 31 exons and spans over 100 kb (FIG. 1A). This gene is the homologue of the human GTF2IRD1 gene on chromosome 7q11.23. To explore the possibility of additional isoforms arising from alternative splicing of this gene, RT-PCR was carried out on cDNA synthesised from RNA from differentiated C2C12 myotubes using the same reverse primers. Under the assumption that the 5′ region was conserved between all the isoforms, forward primers targeted to exon 19 and the coding sequence flanking exon 19 were used, thus only amplifying the variable 3′ region of mMusTRD (FIG. 1B). The PCR products were gel extracted and cloned in pGEM-T Easy vector (Promega) and the positive clones screened by PCR screening and any novel isoforms were sequenced. Three clones were shown to contain the 3′ region of novel isoforms (1α1, 1α4 and 3β3) increasing the number of mMusTRD isoforms to 11 (FIG. 1C).

The coding sequence of the first 18 exons is present in all 11 isoforms without variation, giving rise to a conserved N-terminus. The variation in the C-terminal of the protein arises from alterative splicing of 4 cassettes in addition to two mutually exclusive exons at the extreme 3′ region. Exon 19 constitutes an independent cassette, alternatively spliced in isoforms 3α3 and 3β3, while exon 23 constitutes another, alternatively spliced in isoforms 1α1, 1β1, 3α3, 3β3, 3α5 and 3β5. Exons 21, 22 and 23 constitute a multi exon cassette alternatively spliced in isoform 2α5, and the 3′ region of exon 25, exon 26, exon 27 and the 5′ region of exon 28 constitute another, alternatively spliced in isoforms 1α1, 1β1, 1α4 and 1β4. The two mutually exclusive exons are exons 30 and 31, the isoforms containing exon 30 are termed “β”, while those containing exon 31 are termed “α” (FIG. 1C).

The cassette containing the 3′ region of exon 25, exon 26, exon 27 and the 5′ region of exon 28 constitutes the 3′ region of RD5 and the 5′ region of RD6 (FIG. 1D). When this cassette is alternatively spliced, the resulting coding sequence contains the 5′ end of RD5 and the 3′ end of RD6, which forms a RD completely homologous to RD 6 of the isoforms containing this cassette (FIG. 1D).

The isoforms with either of the multi-exon cassettes alternatively spliced have 5 RDs, similar to the two hMusTRD isoforms, while those with neither alternatively spliced contain 6 RDs, arranged similar to those found in TFII-I (FIG. 2A). These RDs bear approximately 70% homology to those of TFII-I (Roy et al., 1997). The RDs of each protein contain a putative bHLH motif that could be involved in homodimerisation or heterodimerisation and both proteins contain a leucine zipper motif at the extreme N-terminus, also believed to be involved in heterodimerisation. In addition, MusTRD contains a myc-type HLH motif involved in heterodimerisation and each RD also contains an LxxIL motif that may be involved in heterodimerisation with co-activators or co-repressors. MusTRD contains three putative nuclear localisation sequences (NLS), in RD2, RD5 and one after RD6. Each isoform contains at least 2 NLSs while the NLS in RD5 is alternatively spliced, and so is only present in isoforms 3α3, 3β3, 3α5, 3β5, 3α7 and 3β7. The presence of these NLSs is consistent with MusTRDs role as a transcriptional regulator located in the nucleus (FIG. 2B).

### Example 2

### Developmental and Spatial Expression of mMusTRD Isoforms

To determine the developmental and spatial expression patterns of these 11 isoforms, RT-PCR analysis was carried out on RNA isolated from differentiated C2C12 myotubes, 13.5 d.p.c. embryos, and adult VL, soleus and EDL muscles. PCR was carried out using combinations of forward and reverse primers capable of differentiation between isoforms. A forward primer targeted to exon 19, the coding sequence flanking exon 19 or exon 23 in combination with a reverse primer targeted to either exon 30 or 31 were chosen (FIG. 1B). RT-PCR using the forward primer targeted against the coding sequence flanking exon 19 and reverse primer targeted against exon 30 produced a single well defined band around 1.1 kb indicating the presence of mMusTRD3β3 in all sources examined, while the corresponding reaction using the reverse primer targeted against exon 31 produced a single well defined band around 1 kb indicating the presence of mMusTRD3α3 in only myotubes and 13.5 d.p.c. embryos (data not shown). The RT-PCRs using the forward primer targeted against exon 19 and reverse primer targeted to exon 31 resulted in the appearance of several closely grouped bands around 1.2 kb in lanes 2, 3, 4 and 5, and 1.3 kb in lanes 7-11 (data not shown). These reactions could possibly amplify several different isoforms, up to 5 for the reaction employing primer 31 and up to 4 for the reaction employing primer 30, based on those already identified (FIG. 2A). To identify the isoforms present in these heterogenous bands, the PCR products were screened by a method combining cloning and PCR. Identification of positive clones indicated the presence of 3α5 and 3β5 in VL, 3α5, 3α7 and 3β5 in 13.5 d.p.c embryo, 1α1, 1β4, 3α5, 3α7 and 3β5 in soleus and EDL, and 1α1, 1α4, 2α5, 3α5, 3α7 and 3β5 in C2C12 myotubes. To detect the presence of other isoforms, RT-PCR was carried out with the same reverse primers and a forward primer targeted against exon 23 (data not shown). PCR using forward primer 23 and reverse primers 31 produced 880 bp bands in lanes 2-5, indicating the presence of 3α7 in soleus, EDL, 13.5 d.p.c and myotubes, but not in VL. The corresponding PCR using reverse primer targeting exon 31 produced bands at around 950 bp in lanes 7-11, indicating the presence of 3β7 in all sources. In addition to this band, there was also a band running at 650 bp in lane 9 indicating the presence of isoform 1β4 in EDL (data not shown). The isoform 2α5 varies from the other isoforms, in that it lacks RD4 due to the removal of cassette 2, joining exon 20 to exon 24 (FIG. 1C, D). A primer specific to 2α5 was designed, targeting the 3′ region of exon 20 and the 5′ region of exon 24 (5′-CAAGAAATACGATGAGG ATGATG-3′). RT-PCR using this forward primer and the reverse primer targeting exon 31 produced a band running at 850 bp in lanes 2-6, indicating the presence of 2α5 in all sources, though the expression seems to be slightly lower in VL and C2C12 myotubes (data not shown). A summary of the various isoforms found in each source by the combination of RT-PCR and isoform screening are summarised in Table 1.

TABLE 1

| | mMusTRD Isoform | | | | | | | | | | |
|---|---|---|---|---|---|---|---|---|---|---|---|
| | 1α1 | 1α4 | 1β1 | 1β4 | 2α5 | 3α3 | 3β3 | 3α5 | 3α7 | 3β5 | 3β7 |
| C2C12 | † | † | | | † | † | † | † | † | † | † |
| Myotubes | | | | | | | | | | | |
| 13.5 d.p.c | | | | | † | † | † | † | † | † | † |
| embryo | | | | | | | | | | | |
| VL | | | | | † | | † | † | | † | † |
| Soleus | | | † | † | † | | † | † | † | † | † |
| EDL | | | † | † | † | | † | † | † | † | † |

Discussion of Examples 1 and 2.
Structure/Function of the mMusTRD Family

MusTRD is a novel member of the TFII-I family of transcription factors, which contain a signature arrangement of homologous repeat domains containing a bHLH motif. The presence of these bHLH motifs, a leucine zipper, DNA binding domains in addition to the myc-like HLH motif in MusTRD implicate these proteins in DNA-protein interactions, in addition to multiple interactions with other proteins. Both MusTRD and TFII-I have basic regions before the bHLH motif in the first RD, thought to be involved in binding DNA. This is consistent with MusTRDs role as a general transcription factor thought to be involved in coordinating the formation of the basal transcriptional machinery by binding DNA and recruiting other transcriptional regulators. The number of dimerisation motifs suggests that these interactions and MusTRDs role in transcriptional regulation are diverse and complex.

Currently, there are two known hMusTRD isoforms, 944 aa and 959 aa long, the variation between them arising from the alternative splicing of exon 19 (Francke et al., 1999 and Yan et al., 2000), which is also alternatively spliced in the mouse (FIG. 1B). The identification of 11 mouse MusTRD isoforms with highly variable C-terminal ends, suggests that this is a complex family of transcriptional regulators with a diverse range of functions (FIG. 2A). The 3α7 isoform is completely homologous to BEN (Bayarsaihan and Ruddle, 2000), which was isolated from a brain cDNA library and shown to bind the early enhancer region of the Hoxc8 gene. It has also been shown that the C-terminus of Cream1 is capable of binding Rb (Yan et al., 2000). This lends evidence to support the hypothesis that MusTRD plays multiple roles in transcriptional regulation through interactions with a diverse range of co-regulators through the 11 different C-termini.

In addition to facilitating interactions with other proteins, the bHLH domains have been shown to facilitate interactions within the same protein, resulting in varying tertiary structures between the isoforms with 5 RDs and those with 6. In addition to possible variations in affinity of each isoform to other proteins, these mMusTRD isoforms may also interact with each other, producing a large range of homodimers and/or heterodimers with the ability to recognise a diverse range of DNA motifs. The presence of 11 different C-termini indicates the potential for a wide range of tertiary and quaternary structure with affinity for numerous transcriptional co-regulators and DNA motifs, giving rise to a family with a high level of functional variability.

Expression Patterns of the mMusTRD Family

Northern analysis has been previously used to demonstrate the ubiquitous expression of MusTRD. However this technique was unable to distinguish between the 11 isoforms that we have identified. Therefore, RT-PCR was used to differentiate these 11 isoforms showing that they are expressed in varying patterns, temporally and spatially. The expression patterns of several isoforms showed varying degrees of expression in different sources. VL muscle displayed a slightly different expression pattern from soleus and EDL, having lower levels of 2alpha5 and no expression of 3alpha7. Isoform 3alpha3 showed developmental segregation, being only expressed in myotubes and 13.5 dpc embryos, while 1alpha1 and 1alpha4 showed expression exclusively in myotubes. However, 1alpha1 and 1alpha4 showed expression only in adult muscles (soleus and EDL). These isoforms may play specific roles in regulating the expression of specific genes throughout development and in different fibre types, though further biochemical analysis of the effect of each MusTRD isoform on gene expression is required.

These different patterns of expression support the idea of a functionally variable family of transcriptional regulators, capable of acting to control the expression of different genes. hMusTRD1alpha1 is a known transcriptional regulator, having been shown to regulate the expression of Tnl_{slow} (O'Mahoney et al, 1998). Considering the variability at the C-terminal of these 11 mouse isoforms and the variability in expression, we believe that it is likely that they play different roles in regulating the expression patterns of various muscle specific genes, therefore playing a role in fibre type determination.

### Example 3

### Mechanism of Transcriptional Regulation by hMusTRD1

The sequence of hMusTRD1alpha1 contains a number of regions that could be important for its biochemical function. Putative sites for protein dimerisation are present such as a myc-type bHLH motif, phosphorylation sites for protein kinase C (pKC) and protein kinase G (pKG), DNA-binding sites as well as a consensus arginine-lysine (RK)-rich nuclear localisation signal (NLS) motif located in the very C-terminal region of the hMusTRD1alpha1 molecule are schematically depicted (FIG. 5). A diagrammatic representation of the full length hMusTRD1alpha1 protein which is 944aa in length (hMusTRD alpha1₉₄₄ₐₐ) is shown along with a truncated hMusTRD1alpha1 form that is 458aa in length (hMusTRD1alpha1₄₅₈ₐₐ) (O'Mahoney et al., 1998). The repeated domains (RDs) which are 75-93aa in length, are a structural feature present in TFII-I (Roy et al., 1997). These RDs also feature in a series of alternatively spliced mouse MusTRD isoforms. Furthermore, the nomenclature of this emerging family of MusTRD proteins, in part, depends on the appearance and order of these RDs in each isoform (see Examples 1 and 2).

MEF2C Activates and hMusTRD1α1 Represses Transcription Through the hTnl_{slow} USE

The transcriptional regulation of the hTnl_{slow} upstream enhancer element (USE) via MEF2C and hMusTRD1α1 was investigated initially in C2C12 muscle cultures. Expression vectors encoding hMusTRD1α1 and MEF2C were co-transfected into C2C12 cells with luciferase reporter gene constructs driven by either the intact hTnl_{slow} USE or USE-B1b containing a mutation in the hMusTRD1α1 binding site within the B1 region (FIG. 15A). MEF2C elicited an approximate 2-fold increase in hTnl_{slow} USE transcriptional activity in C2C12 cells (FIG. 15B). In contrast, hMusTRD1α1 repressed the basal activity mediated by the hTnl_{slow} USE by approximately 2-fold. To assess transcriptional effects of hMusTRD1α1 on MEF2C-mediated transactivation, hMusTRD1α1 and MEF2C expression constructs were co-transfected with the wild-type hTnl_{slow} USE. hMusTRD1α1 repressed the MEF2C-mediated transcriptional activation of the hTnl_{slow} USE by approximately 3-fold. This experiment also demonstrates that basal transcriptional activity was mediated by the B1 element, since there is a 36-fold reduction in activity upon mutation of the GATTAA core sequence. Furthermore, mutation of this core sequence resulted in a 68-fold reduction of MEF2C-mediated induction of the hTnl_{slow} USE demonstrating that this site is necessary for MEF2C-mediated transcriptional activation.

hMusTRD1α1-mediated repression of the USE could be due to an intrinsic property of the protein or to the modification of a myogenic transcriptional enhancing factor such as MEF2C or other MusTRD isoforms. To discriminate these possibilities, expression studies were conducted in Cos-7 cells that express negligible amounts of MusTRD transcripts in contrast with C2C12 cells that express a number of MusTRD isoforms, including the mouse orthologue of hMusTRD1α1 (data not shown). In addition, the B1 region of the USE was used that contains the Inr-like element, but lacks the MEF2 binding site. The B1 region was trimerized and linked to a heterologous promoter driving luciferase to achieve a sufficient level of expression in Cos-7 cells (FIG. 15A). hMusTRD1α1 was co-transfected together with a construct bearing either the B1 or B1b region. hMusTRD1α1 repressed basal transcriptional activity by approximately 3-fold (data not shown). Mutation of the core GATTAA sequence in the B1b version, resulted in a 4-fold loss of basal transcriptional activity in Cos-7 cells. These data demonstrate that hMusTRD1α1-mediated repression can occur in the absence of MEF2C and its binding site. These results also suggest that an unidentified factor binding to B1 is needed for MEF2C activation and repression by hMusTRD1α1.

hMusTRD1α1 Contains two DNA Binding Domains

In order to locate the region(s) of hMusTRD responsible for DNA-binding and transcriptional repression, we examined the functional capabilities of a deletion series of hMusTRD1α1 cDNAs. As a first step in this process we demonstrated that in vitro translated full length hMusTRD1α1 (hMusTRD1α1₉₄₄ₐₐ) binds to the Inr-like element within the B1 region of the hTnl_{slow} USE (data not shown). Oligonucleotides containing either a trimerized B1 or B1b element were incubated with in vitro translated hMusTRD1α1₉₄₄ₐₐ protein. A protein-DNA complex formed when the B1 element was used as a probe, but did not form when the hMusTRD1α1 cognate binding site was mutated in the B1b element. The presence of hMusTRD1α1 in the largest complex was demonstrated using an α-MusTRD1α1₁₋₂₀ₐₐ antibody directed against the first 20aa of hMusTRD1α1 (data not shown). The specificity of the antibody was shown by Western immunoblotting (data not shown). A loss of formation of the complex occurred in the presence of the antibody demonstrating that it interferes with hMusTRD1α1 binding to the B1 element.

Truncated versions of hMusTRD1α1 were generated by progressive C-terminal deletion of putative regulatory regions as well as an N-terminally deleted peptide lacking RDs 1 and 2 (ΔN444aa) (FIG. 6). PCR products were subcloned into the pcDNA3.1myc/his expression vector for in vitro translation of proteins that were 350aa, 458aa, 500aa, 564aa, 689aa and 786aa in length. These were used in subsequent EMSA and transfection assays. hMusTRD1α1₃₅₀ₐₐ, containing RD1 only, was unable to bind the B1 element (data not shown). In contrast, hMusTRD1α1₄₅₈ₐₐ clearly binds DNA demonstrating that a DNA binding domain (DBD1) is located in the N-terminus between 351-458aa. The presence of a second DNA binding domain (DBD2) between 544-944aa was demonstrated by the binding of hMusTRD1α1NΔ₄₄₄ₐₐ that lacks DBD1. hMusTRD1α1 ₇₈₆ₐₐ binds to the B1 element most avidly as indicated by a band of greater intensity in comparison with the other deletions. This suggests that hMusTRD1β1₇₈₆ₐₐ may contain both DBDs and that DBD2 may exist in 544-786aa. The binding activities of hMusTRD1α1₄₅₈ₐₐ, hMusTRD1α1₅₆₄ₐₐ, hMusTRD1α1₇₈₆ₐₐ, and hMusTRD1α1NΔ₄₄₄ₐₐ were lost upon mutation of the B1 element, further indicating that the GATTAA site is the core area for interaction for both DBD1 and DBD2. These data demonstrated that hMusTRD1α1 contains two DBDs.

hMusTRD1α1 can Repress in the Absence of DNA Binding

The RDs that consist of 75-93aa are a feature in common with TFII-I (Roy et al., 1997). Each RD contains an LxxIL motif that is found in most coactivators required for hormone-dependent or -independent nuclear hormone receptor interactions (Sauve et al., 2001; Li et al., 2001). In order to determine if different regions of hMusTRD1α1 possess differential transcriptional activities and to study the functional significance of the two DBDs, the hMusTRD1α1 C-terminal truncation and N-terminal deleted constructs were co-transfected into Cos-7 cells along with a luciferase construct driven by the trimerized B1 region. All constructs, including hMusTRD1α1₃₅₀ₐₐ that is incapable of binding the B1 region, repressed activity by 70% (FIG. 16). These data demonstrate that hMusTRD1α1 can repress through a mechanism that is independent of direct DNA binding.

Mammalian one-hybrid assays in Cos-7 cells were used to confirm that hMusTRD1α1 can repress in the absence of binding to its cognate DNA binding site. Fusion proteins containing the DBD of GAL4 linked to hMusTRD1α1₉₄₄ₐₐ were constitutively expressed in Cos-7 cells in combination with a luciferase reporter gene construct driven by a trimerized GAL4 DNA binding site (FIG. 17A). Reporter gene activity was determined and expressed as fold repression over basal GAL4DBD activity (FIG. 17B). hMusTRD1α1₉₄₄ₐₐ was able to repress the basal activity by approximately 3.5-fold without directly contacting the DNA via the GAL4 DBD. The repressive capability of hMusTRD1α1₉₄₄ₐₐ was tested against the repression domains (RDs) present in the N-terminal portion of the potent co-repressor NcoR (Horlein et al., 1995). Repression was similar with both factors. Taken together, these results validate the co-transfection results shown in FIG. 16 and demonstrate that hMusTRD1α1₉₄₄ₐₐ can repress without binding to its cognate DNA binding site. In addition, they show that the repressive activity of hMusTRD1α1 is comparable to a known repressor molecule that functions without directly binding DNA.

hMusTRD1α1 and NCoR can Physically Interact In vivo and In vitro

Co-immunoprecipitation assays were performed to test for protein-protein interaction between hMusTRD1α1 and NCoR in vivo. hMusTRD1α1₃₅₀ₐₐ was used since it is the smallest peptide that lacks a DBD and can repress in the absence of DNA binding. An antibody that recognises the C-terminal region of NCoR was used to successfully co-immunoprecipitate a protein complex containing full length NCoR and hMusTRD1α1₃₅₀ₐₐ from Cos-7 cells (data not shown). GST pull-down assays were used to demonstrate in vitro interactions between ³⁵S-labeled hMusTRD1α1₃₅₀ₐₐ and the portion of NCoR that contains the nuclear receptor interaction domains (Ids) responsible for interaction with other proteins/transcription factors, GST-NCoR₁₆₄₉₋₂₄₅₃ (data not shown). These protein-protein interaction data confirmed that hMusTRD1α1 can interact with NCoR via a mechanism that is independent of DNA binding. Taken together, these findings suggest that NCoR and hMusTRD1α1 could co-operate to mediate the transcriptional repression of the hTnl_{slow} gene.

hMusTRD1α1 abrogates MEF2C Binding to the TnhI_{slow} USE 3′MEF2 Site Through Direct Interaction

EMSA was used to examine how hMusTRD1α1 can repress MEF2C-mediated transactivation. Using oligonucleotides containing either a wildtype or a mutated 3′MEF2 binding site from the USE, MEF2C binding was demonstrated. This binding was gradually competed off with increasing concentrations of hMusTRD1α1 (data not shown). This result explains why MEF2C-mediated transactivation was blocked by hMusTRD1α1 in the co-transfection analysis.

GST pull-down assays were used to determine if hMusTRD1α1-mediated repression of MEF2C transactivation could involve direct interactions between the proteins. Phosphorimager detection revealed direct binding of ³⁵S-labeled MEF2C and bacterially expressed GST-hMusTRD1α1₉₄₄ₐₐ (data not shown). Taken together, EMSA and pull-down experiments suggest that MEF2C and hMusTRD1α1 interact to form an abortive complex that could prevent MEF2C from interacting with its cognate binding site within the Tnl_{slow} USE.

Discussion of Example 3

There are several mechanisms of obtaining gene repressive effects, these include 1) direct, DNA binding of a nuclear transcription factor to a DNA docking site, 2) competitive interaction of a co-repressor molecule operating in trans to take an activator molecule away from its enhancer site and 3) interaction of a co-repressor molecule with a DNA-bound activator to cause direct down-regulation of gene activation via DNA-protein-protein complex formation.

The data presented in this example address key aspects of the transcriptional regulation of hTnl_{slow} and potentially, of slow fiber-specific genes in general. The Inr-like element within the hTnl_{slow} USE is an essential regulatory element involved in both transcriptional activation and repression. The data are consistent with a model whereby MEF2-mediated transcriptional activation occurs through the 3′MEF2 binding site of the USE and requires occupancy of the Inr-like element by unknown activating factors. We demonstrate that mutation of the lnr-like element prevents MEF2C-mediated activation via the USE. This key finding shows that USE function relies on cooperation between proteins binding at the Inr-like site and MEF2 proteins.

hMusTRD1α1 is found to repress activity of the USE and this may be achieved by several means. Firstly, hMusTRD1α1 binds to the Inr-like element by virtue of 2 sequence-specific DNA binding domains and must therefore compete with the activating factor for occupancy of the binding site. Secondly, we have demonstrated a direct interaction between hMusTRD1α1 and the nuclear receptor co-repressor NcoR. In addition, hMusTRD1α1 can mediate repression via a DNA-independent mechanism that utilises its ability to interact directly with NCoR and MEF2C. It could either sequester MEF2 or hinder interactions between MEF2 and activation factors.

These data support mechanisms for transcriptional activation and repression of the hTnlslow that are dependent on an intact Inr-like element in combination with the 3′MEF2 binding site. Transcriptional activation is achieved when the B1 element is occupied by an enhancing factor that interacts with MEF2C bound to the 3′MEF2C site (FIG. 18A). Repression is achieved by hMusTRD1α1 in a DNA-dependent or -independent manner (FIG. 18B). hMusTRD1α1 can prevent the proper interaction of MEF2C with the enhancing factor either by sequestering MEF2C in conjunction with NCoR or by occupying the Inr-like element and preventing the binding of the enhancing factor.

A growing number of signaling pathways have been found to converge on the MEF2 proteins, thereby regulating their essential role in transcriptional control of muscle-specific genes. MEF2C and MEF2A are substrates for p38 MAP kinase and MEF2C is a substrate for BMK1/ERK5. Other pathways involve calcium sensitive proteins including calcineurin, which activates MEF2 by direct dephosphorylation and Ca²⁺-calmodulin-dependent protein kinase CaMK, which activates MEF2 by alleviating the repression imposed by the HDACs. Signaling via Ca²⁺ dependant pathways has recently been proposed as a potential means of fiber-type adaptation since sustained patterns of nerve stimulation elevate intracellular calcium levels and recent experiments in mice support this hypothesis. Additionally, the transcriptional co-activator PGC-1α has been found to elicit slow fiber-specific gene expression, as well as mitochondrial biogenesis, through interaction with MEF2. Therefore, MEF2 is a key factor involved in translating endurance activity and electrical activity-mediated changes in intracellular Ca²⁺ levels within muscle fibers into muscle gene transcriptional activity. It is clear that by disrupting the transcriptional activation capacity of MEF2C in the USE of Tnl_{slow} as demonstrated in this study, hMusTRD1α1 acts at a nodal point in the regulation pathway of slow fiber-specific genes.

The mode of hMusTRD1α1 repression may rely on several intrinsic properties. Firstly, hMusTRD1α1 was found to interact with the B1 element via two DBDs. Truncated versions of hMusTRD1α1 containing either or both DBDs bind specifically to the B1 enhancer element of the USE through the core binding motif GATTAA defined by O'Mahoney et al., (1998). In all instances, mutation of this sequence to GATatc prevented binding. Furthermore, the α-hMusTRD1α1₁₋₂₀ₐₐ antibody was capable of directly blocking hMusTRD1α1 DNA-binding activity, further verifying the specificity of this DNA-protein complex.

The interaction of hMusTRD1α1 with the B1 enhancer element occurs via two, functional DBDs that are independently located at both N- and C-terminal ends of the molecule. Deletion analysis has indicated the presence of an N-terminal DBD1 in RD2 and also the presence of a C-terminal DBD2 in RD4, located in between 351-458aa and between 544-944aa, respectively. These regions are rich in basic amino acid residues, which are typically involved in either DNA- or protein-dimerisation. Upon review of the database search for putative DNA binding domains, we propose that two basic amino acid-rich regions at 408-420aa and 738-765aa are likely to correspond to DBD1 and DBD2, respectively. This finding is unusual since, for most transcription factor families, the region for DNA interaction is typically grouped to one defined area, usually the N-terminal part of the protein as is the case for the nuclear hormone receptor superfamily of transcription factors.

The direct association of hMusTRD1α1 with the nuclear receptor co-repressor N-CoR demonstrates a second mode of repression. N-CoR functions as a co-repressor not only for nuclear hormone receptors but also for multiple classes of transcription factors. The multiple, amino-terminal repression domains mediate interactions via mSin3 with large complexes containing class I histone deacetylases (HDACs) or, by direct association, with class II HDACs, thereby modifying chromatin structure through histone hypoacetylation. Co-immunoprecipitation studies recently revealed an association between hMusTRD1α1 and the class I histone deacetylase HDAC3. It is possible that this interaction is mediated by an N-CoR dependent mechanism.

We believe that, in general, it is sequences present in the C-terminus that allow MusTRD isoforms to regulate transcription through these diverse mechanisms by providing differential interactions with proteins present in different cellular environments.

In summary, a direct, DNA-dependent pathway for repression mediated by hMusTRD1α1 has been presented. This DNA-dependent interaction between hMusTRD1α1 and the B1 enhancer element in the hTnl_{slow} USE, is mediated via two functional DBDs which are novel and positioned at opposing regions of the molecule and illustrates an example of direct transcriptional repression. In addition, an intrinsic functional repressive potential by hMusTRD1α1 has also been demonstrated, which highlights an additional feature of this molecule to mediate repressive effects via indirect mechanisms.

### Example 4

### Expression of MusTRD in Mouse Muscles

We investigated the expression and function of hMusTRD1α1 -like proteins in mouse muscles. An antibody targeted to an N-terminal peptide sequence of human hMusTRD1α1 was generated and found to react specifically with wild-type (wt) hMusTRDα1 ectopically expressed in COS-7 cells (FIG. 7A, lane 3). hMusTRD1α1-like proteins were expressed in both slow and fast myonuclei of mouse soleus muscle (FIGS. 7B-D). Immunostaining for dystrophin, a component of the plasma membrane, confirmed expression of hMusTRD1α1-like proteins within myofiber nuclei and not in quiescent satellite cells.

Nuclear extracts of mouse soleus and extensor digitorium longus (EDL) muscles have hMusTRD1α-containing protein complexes with affinity for the Tnl_{slow}USE Inr-like element (Tnl_{slow}USE-B1), similar to those previously described in rat muscles (FIG. 7E, lanes 1, 2, 5 and 6; O'Mahoney et al. 1998). These protein-DNA complexes were abolished by a mutation in the hMusTRD1α1 Inr-like interaction site (Tnl_{slow}USE-B1b; FIG. 7E, lanes 3, 4, 7 and 8).

The expression of mouse hMusTRD1α1-like mRNA transcripts in the developing embryo was assessed by Northern blot analysis. A cDNA clone with high homology to human hMusTRD1α1 was identified from a mouse quadriceps cDNA library (unpublished work). Sequences corresponding to the RD1-RD2 intervening region of human hMusTRD1α1 (nucleotides 675-1066) were used to generate a probe for use in Northern blot analysis. Three transcripts of approximately 3.6, 4.4 and 5.9 were detected as early as 7 dpc (FIG. 7F). The sizes of the three transcripts correspond with those observed for tissue mRNA expression of BEN, the recently described mouse MusTRD-TFII-I family member (Bayarsaihan et al. 2000). Previous analysis of human MusTRD mRNA showed that the 3.3 kb transcript is the most abundant (O'Mahoney et al. 1998), similarly, the 3.6 kb species is predominant in the mouse. Hence, the mouse expresses a homologue(s) of human MusTRD1α1 and mouse muscles contain Tnl_{slow}USE-B1 binding proteins.

### Example 5

### Regulation of Fast and Slow Fiber-specific Promoters/Enhancers by hMusTRD1α1, ΔhMusTRD1α1 and mMusTRD Isoforms

In order to determine the potential capabilities of the MusTRD isoforms to regulate slow and fast fiber-specific genes, we examined their effect on the expression of representative slow and fast fiber-specific contractile protein gene promoters/enhancers. Expression plasmids containing hMusTRD1α1, ΔhMusTRD1α1 and mMusTRD isoforms 3α7, 1β1, and 3β7 were co-transfected with luciferase expression plasmids containing promoter/enhancer elements from the fast fiber-specific MHCIIB gene (di Maso et al, 2000) and slow fiber-specific MHCl_{slow} (Wright et al, 2001), MLC2_{slow} (Esser et al, 1999) and Tnl_{slow} (O'Mahoney et al, 1998) genes into the myogenic cell line C2C12. hMusTRD1α1 and ΔhMusTRD1α1 acted similarly in repressing the transcriptional activity of MHCl_{slow} and Tnl_{slow}, but had little to no effect on MLC2_{slow} and MHCIIB expression (FIG. 8A). In contrast, mMusTRD3α7, 1β1, and 3β7 had little to no effect on any of the regulatory elements with the exception of 1β1 which increased MHCIIB expression in this assay (FIG. 8B). These results demonstrate that MusTRD isoforms have the potential to act on fiber-specific gene sequences to either repress or activate transcription.

### Example 6

### Generation of a Dominant Negative MusTRD (ΔMusTRD1α1) Mouse Model

We predicted that a mutant protein truncated at amino acid 458 (ΔhMusTRD1α1; FIG. 7A, lane 2) would function as a dominant negative for wild-type (wt) hMusTRD1α1 and indeed all splice products from the MusTRD gene. To assess this, we determined that wt hMusTRD1α1, but not ΔhMusTRD1α1, exhibited autologous transactivation when fused to the gal4 DNA binding domain in the yeast two-hybrid assay (FIG. 9A). Both wt hMusTRD1α1 and ΔhMusTRD1α1 interacted with the Tnl_{slow}USE-B1 element, but not the Tnl_{slow}USE-B1b mutant, in the yeast one-hybrid assay (FIG. 9B). ΔhMusTRD1α1 didn't bind as efficiently as wt hMusTRDα1. In addition, wt hMusTRD1α1 and ΔhMusTRD1α1 formed homodimers and heterodimers (FIG. 9C). Wild-type hMusTRD1α1 is a nucleophilic protein and ΔhMusTRD1α1 retained this function when transfected in COS-7 cells (data not shown). In summary, ΔhMusTRD1α1 exhibited DNA binding capacity and lacked transactivation function demonstrating its capacity as a dominant negative competitor of endogenous hMusTRD1α1.

To investigate the role of a reduced level of functional MusTRD in mediating the myopathic features of WBS, we generated transgenic mice that express ΔhMusTRD1α1 under the control of the human skeletal actin (HSA) promoter (FIG. 10A; Muscat and Kedes, 1987). This promoter directs transgene expression in developing myotubes from 7.5 dpc (McLeod and Hardeman, unpublished observation), coincident with the expression of the endogenous MusTRDs (see FIG. 7F), and is active in myofibers during perinatal and postnatal development. Five independent transgenic lines were analysed and ΔhMusTRD1α1 mRNA transcript and protein levels were detected in muscles (FIG. 10B). There was no correlation between ΔhMusTRD1α1 mRNA and protein levels (FIG. 10C).

Kyphoscoliosis, Joint Contractures and Growth Retardation in ΔhMusTRD1α1 Mice

All transgenic progeny from ΔhMusTRD1α1 lines 10, 11, 29 and 70 developed the musculo-skeletal defect, kyphoscoliosis, seen as a curvature in the spine by X-ray analysis as early as 4 weeks of age (FIGS. 10D,E). This deformity arises from compression of the cervico-thoracic vertebrae, presumably due to weakness in the paravertebral muscles. ΔhMusTRD1α1 transgenics tended to be sluggish and less active than their wt littermates, and exhibited an abnormal waddling gait. In addition, they showed weakness in their hindlimbs, which tended to splay (FIG. 10F).

ΔhMusTRD1α1 mice were significantly smaller than their wt littermates at all ages (FIG. 10G,H). At weaning transgenic progeny of lines 10 and 11 were 21% and 18% smaller than their wt littermates. The rate of growth during the period of exponential growth (3-7 weeks) did not differ between transgenic and wt mice, indicating ΔhMusTRD1α1 mice were not malnourished (FIG. 10G). ΔhMusTRD1α1 mice continued to grow up to 12 months of age, but remained 22% and 30% (lines 10 and 11, respectively) smaller than wt littermates (FIG. 10H).

Myofiber Specialisation, Maturation and Growth Hypertrophy is Disrupted in ΔhMusTRD1α1 a Mice

Consistent with low body weights, the muscle cross-section areas of ΔhMusTRD1α1 mice were reduced relative to wt (FIGS. 11A-D). Fiber composition, as defined by myosin heavy chain (MHC) isoform expression, was altered. In wt soleus muscle of the B6D2 strain, approximately 45% of myofibers express MHC type I (MHCl_{slow}), a marker for slow myofibers, and 55% of fibers express MHC type IIA (MHCIIA_{fast}), a marker for oxidative fast-twitch fibers (FIG. 11A,B). In contrast in ΔhMusTRD1α1 soleus muscles, MHC-I_{slow} was diminished or absent and all fibers appeared to express MHC-IIA_{fast} (FIGS. 11A,C,D). Transgenic soleus muscle cross-section areas were 50%, 51%, 27% and 20% smaller than wt littermates, and fiber diameters decreased by 33%, 38%, 19% and 23%, for lines 29, 11, 10 and 70, respectively (FIGS. 11E,F). Lines 29 and 11, which had the highest protein expression levels, exhibited the most profound reduction in muscle area and fiber diameter, suggesting a gene dose-dependent effect. The number of myofibers in the soleus of progeny of lines 29, 11, 10 and 70 were 87%, 97%, 82% and 98% of wt, respectively; however, the differences were significant only for lines 29 and 10 (FIG. 11G). Note that the reduction in muscle mass in the different lines correlated with the level of ΔhMusTRD1α1 protein expression (FIG. 11E; FIG. 10C).

The EDL, a predominantly fast fiber-containing muscle (80% glycolytic MHC-IIB_{fast}, 15% MHC-IIA_{fast}, 5% MHCl_{slow}), was also smaller than wt and had reduced fiber diameters (FIGS. 11H,I). All myofibers of the transgenic EDL muscle co-expressed MHC-IIA_{fast} and MHC-IIB_{fast} and lacked expression of MHC-I_{slow} (data not shown). ΔhMusTRD1α1 EDL muscles showed features characteristic of muscle undergoing degeneration and regeneration. Fiber degeneration was associated with an increase in cell necrosis (cytoplasmic condensation), vascularisation and macrophage infiltration, as validated by an aαnaphthol acetate esterase activity assay (FIG. 11J,K; Yam et al. 1971; Bhatia et al. 1994). Fiber regeneration correlated with a slight increase in myofiber number and a corresponding increase in the proportion of centrally nucleated myofibers (FIGS. 11L,M). Fiber degeneration was present in muscles containing predominantly fast myofibers, such as the tibialis anterior, gastrocnemius, plantaris and flexor digitorum fibularis muscles, but not in slow fiber predominant muscles such as the soleus and popliteus muscles.

To investigate the possibility that ΔhMusTRD1α1 myofibers are developmentally delayed, we tested for expression of the neonatal/perinatal isoform of MHC (MHCₙₑₒ), which is expressed only in immature myofiber. Protein extracts of diaphragm and soleus muscles from 7 week-old mice were subjected to high resolution gel electrophoresis followed by silver staining or Western blotting (FIG. 12A). Transgenic diaphragm and soleus muscles lacked MHCl_{slow} expression. MHCₙₑₒ, which is expressed in limb muscles at postnatal day 1 (PND1), was present in ΔhMusTRD1α1, but not wt soleus extracts. This was confirmed by Western blotting using an antibody to MHCₙₑₒ.

hMusTRD1α1 and ΔhMusTRD1α1 Repress Most Slow Fiber-specific Genes in Soleus and EDL Muscles

Consistent with the absence of MHCl_{slow} expressing myofibers in ΔhMusTRD1α1 muscles, slow isoforms of other myofibrillar gene families were also down-regulated (FIG. 12B). Myosin light chain-1 slow A (MLC1_{slowA}), Tnl_{slow}, and α-tropomyosin slow (αTm_{slow}) were expressed in wt soleus but not EDL muscles. Expression of these isoforms was down-regulated in ΔhMusTRD1α1 soleus muscles; however, the expression of MLC2_{slow} was the same as in wt muscles.

Consistent with repression of Tnl_{slow} transcript accumulation, ΔhMusTRD1α1 repressed Tnl_{slow}USE-mediated expression in vivo. Hemizygotic −2000HSA:ΔhMusTRD1α1 mice were mated with transgenics homozygotic for the Tnl_{slow}USE-95X1 nucZ transgene locus (FIG. 13A). In soleus from two week-old Tnl_{slow}USE-95X1nucZ mice, expression of the reporter was restricted to MHC-I_{slow}-containing fibers, as described previously (FIG. 13B,C). The presence of ΔhMusTRD1α1 repressed both the number of myonuclei containing β-galactosidase and the level of expression within individual nuclei (FIGS. 13D,E). The level of reporter activity was reduced in soleus but not EDL muscle extracts, confirming that MusTRD is required for slow fiber-specific expression of Tnl_{slow} (FIGS. 13F,G).

hMusTRD1α1 and ΔhMusTRD1α1 Have Differential Effects on Slow Fiber Gene Expression in Cural Muscles

An examination of the entire field of crural muscles revealed that hMusTRD1α1 consistently acts to repress slow fibre gene expression in all muscles as evidenced by the marked reduction in MHClslow positive fibres in the slow fiber rich soleus and plantaris adjacent lateral gastrocnemius (PALG) (data not shown). ΔhMusTRD1α1 acts in a similar manner in the soleus; however, in stark contrast it elicits MHClslow expression in the remainder of the crural muscles with the exception of the EDL (data not shown). The most dramatic effect is in the tibialis anterior muscle in which 40% of the fibres express MHClslow compared with none in the wt mouse. The differential effect of ΔhMusTRD1α1 on MHClslow expression in different muscles suggests that the combination of factors that regulate fibre-specific gene expression in slow and fast fibres may differ amongst muscles and that a disruption of the balance of factors can be achieved by ΔhMusTRD1α1.

Discussion

The identification of transcription factors that mediate differential gene expression in slow- or fast-twitch myofibers is essential to understanding the mechanisms underlying muscle plasticity, in particular myofiber conversion in congenital myopathies, nerve or muscle injury, exercise and ageing. We previously established that an isoform of the transcription factor MusTRD, previously named MusTRD1and renamed hMusTRD1α1 in this study, is present in skeletal muscle and interacts directly with a regulatory element in the enhancer of the Tnl_{slow} gene (O'Mahoney et al. 1998). Here we show that the gene that encodes MusTRD (WBSCR11, GTF2IRD1, GTF3) gives rise to at least 11 isoforms that result from alternative splice products with the variability amongst isoforms residing in the 3′ carboxy terminus of the protein. These isoforms are present in, but may not be restricted to, the muscle cell line C2C12, and embryonic and adult muscles. In general, both hMusTRD1α1 and ΔhMusTRD1α1 act as repressors of slow fiber-specific genes, with the exception of MLC2slow, in cell culture and the classical slow fiber mouse muscle, the soleus. However, f considerable interest, the dominant negative ΔhMusTRD1α1 had a differential effect on fibre-specific gene expression in different muscles of the hindlimb, in contrast with hMusTRD1α1 which consistently repressed the slow fiber phenotype. The MusTRDs also affect growth hypertrophy since muscle fiber diameter was significantly reduced in both types of MusTRD transgenic mice.

The presence of a myopathic feature in WBS suggests that an important regulator of myogenesis localises to the deletion region on chromosome 7q11.23. The gene encoding MusTRD (WBSCR11; GTF2IRD1, GTF3) localises to this region, however its contribution to the various components of the WBS phenotype is unknown. The physical features of the ΔhMusTRD1α1 mouse model implicate MusTRD in mediating the myopathic aspects of the WBS phenotype. ΔhMusTRD.1α1 transgenic mice developed kyphoscoliosis and joint contractures. These mice were sluggish and less active than their wt littermates, and exhibited an abnormal waddling gait due to weakness in their hindlimbs. Similarly, WBS patients exhibit muscle fatigue and hypotonia. Approximately 20% of patients develop kyphoscoliosis, while joint contractures, which affect approximately 50% of patients, can often be severe enough to hinder mobility and normal activities (Voit et al. 1991).

A distinguishing clinical feature of WBS is growth retardation, characterised by birth weights and length less than the 10th percentile, delayed growth in infancy, a growth spurt in puberty, and low ultimate adult height. In comparison, ΔhMusTRD1α1 mice were growth retarded during post-natal development and remained up to 30% smaller than wt littermates in adulthood. The similarities between the ΔhMusTRD1α1 mouse model and the clinical features of WBS suggest that the physical defects and growth retardation in WBS may have a skeletal muscle involvement and that haploinsufficiency of the MusTRD gene may contribute to some of these features.

Muscle mass is a significant proportion of the body weight of mice (approximately 70%). Hence, we postulated that a defect in muscle growth may contribute to the growth retardation in ΔhMusTRD1α1 mice. During the first two weeks following birth of wt mice, newly formed myofibers undergo maturation and growth hypertrophy, a process leading to an increase in the cytoplasm to nuclei ratio, and, ultimately, in muscle mass. In ΔhMusTRD1α1 transgenics, muscle mass and fiber diameter of all hindlimb muscles were reduced. Histomorphological analysis revealed that myofiber diameter, but not myofiber number, is reduced in the soleus muscles of several ΔhMusTRD1α1 transgenic lines. The degree of myofiber hypotrophy correlated with the level of ΔhMusTRD1α1 protein expression, suggestive of a gene dose-dependent effect. Hence, a defect in myofiber growth hypertrophy, not myofiber number, accounts for the reduced muscle mass in ΔhMusTRD1α1 mice. In comparison, WBS patients are reported to exhibit increased variability in myofiber size with both fiber atrophy and hypertrophy (Voit et al. 1991). Similarly, we observed myofiber atrophy and degeneration, a hallmark of dystrophic muscle, in some muscles of ΔhMusTRD1α1 mice. Interestingly, this was a feature of muscles containing predominantly fast myofibers, but not of slow-fiber containing muscles. This muscle-specific difference may reflect a fast myofiber-specific function of hMusTRD1α1 or related proteins, distinct from that in slow myofibers.

In addition to its requirement for myofiber growth hypertrophy, hMusTRD1α1 appears to be affecting maturation of differentiated myotubes. This is evidenced by the gene expression profile of ΔhMusTRD1α1 muscles, which reflects developmental delay. In the wt mouse, expression of the MHCₙₑₒ isoform appears after 12.5 dpc when secondary myotubes are forming. From 15 dpc, MHCₙₑₒ expression becomes restricted to secondary myotubes and those primary myotubes that are destined to become fast fibers in the adult. MHCₙₑₒ is the most abundant MHC isoform at birth then declines around 3-5 days postnatal, coincident with the onset of myotube specialisation and appearance of mature MHC isoforms. In contrast, MHCₙₑₒ expression persisted in the adult muscles of ΔhMusTRD1α1 mice. Furthermore, there was coexpression of MHC-IIA_{fast} and MHC-IIB_{fast} within a single myofiber. Coexpression of MHC isoforms is observed in immature myofibers prior to the establishment of a specific fiber phenotype and in myofibers undergoing conversion. Hence, the altered distribution and abundance of MHC isoforms suggests that the process of myofiber specialisation to an adult phenotype appears to be prevented or delayed in ΔhMusTRD1α1 muscles.

Both hMusTRD1α1 and a dominant negative form of the protein, ΔhMusTRD1α1, repressed at least four slow-fiber specific promoters in co-transfection studies, but did not affect the expression of MLC2slow. In addition, slow isoforms of myofibrillar genes were repressed in the soleus of ΔhMusTRD1α1 transgenic muscles with the exception of MLC2slow. Regulation of the developmental and fast forms of MHC differs; MHCIIB is not affected, and MHCIIA and neonatal are upregulated. The absence of MHC-I_{slow} expressing myofibers in ΔhMusTRD1α1 muscles suggests that there is also a defect in regulation of the slow myogenic phenotype. Consistent with this possibility, the slow isoforms of three other myofibrillar genes were down-regulated. MLCl2_{slowA}, which is expressed in both primary and secondary myotubes in the developing embryo and becomes restricted to slow fibers in the adult, was down-regulated in ΔhMusTRD1α1 muscles. Expression of Tnl_{slow} and αTm_{slow}, both of which are expressed in developing and adult slow myofibers, were also down-regulated in ΔhMusTRD1α1 soleus muscles. Hence, components of the slow myogenic phenotype appear to be repressed in ΔhMusTRD1α1 mice. In contrast, MLC2_{slow}, an adult isoform that is responsive to innervation status, was expressed at similar levels in ΔhMusTRD1α1 as in wt soleus muscles, suggesting that gene markers of the slow myofiber phenotype are not coordinately regulated. Whether loss of slow myofibers results from a defect in myotube commitment to the slow phenotype or from a defect in the maturation of established slow myofibers is unclear. The activation of MHCIIb by mMusTRD1β1 indicates that different isoforms of MusTRD have differential activating or repressing capabilities on fast and slow fiber-specific genes. Taken together, these results suggest that hMusTRD1α1 and related proteins are important regulators of myogenesis and the establishment of the fast and slow myogenic profiles. These results suggest that the slow fiber phenotype is not regulated/established by a single factor or group of factors. hMusTRD1α1 and ΔhMusTRD1α1 regulate transcription of the Tnl_{slow} gene in vivo through an interaction with an Inr-like element. This raises the question of whether hMusTRD1α1 is a general regulator of slow myogenic genes and whether MusTRD binding elements are present in the MHC-I_{slow} MLC1_{slowA} and αTm_{slow} genes. The Tnl_{slow}USE also contains MEF2 binding sites and E-box consensus sequences. MEF2 and the calcium-dependent calcineurin-NFAT pathway have been implicated in the induction and/or maintenance of the slow myofiber type. However, deletion of either MEF2 and/or NFAT binding site in the rat Tnl_{slow}USE was not sufficient to abolish slow-myofiber specific gene expression (Calvo et al. 1999). In comparison, disruption of the hMusTRD1α1 Inr-like binding element significantly reduced Tnl_{slow}USE-mediated gene expression in slow myofibers (O'Mahoney et al. 1998) and disruption of MusTRD function repressed expression of slow myogenic genes in this study. However, since TFII-I can integrate signals from second messengers, and bind to serum response factors and HLH factors to regulate transcription, it is possible that MusTRD may act cooperatively or in concert with MEF2, NFAT or MyoD family members to regulate the slow myogenic phenotype.

The possibility that over-expression of ΔMusTRD is disrupting the function of other MusTRD isoforms or unknown TFII-I-MusTRD family members is also a consideration. Recently, homo- and heterodimerisation between isoform variants of TFII-I has been shown to have differential effects on the regulation of a TFII-I target gene. Similarly, MusTRD can form homodimers and may potentially heterodimerise with other MusTRD isoforms. Therefore, ΔMusTRD may exert its effects, in part, through interaction with other MusTRD isoforms. However, whether MusTRD isoforms co-localise to muscle cells remains to be determined.

Disruption of MusTRD function in the mouse causes a myopathic phenotype. There are at least four mechanisms by which a myopathic or dystrophic feature can be induced in mice: 1) disruption of cytoskeletal genes, such as utrophin with dystrophin, 2) disruption of connective tissue genes, such as collagen VI or laminin, 3) disruption of myogenic regulatory factors (MRF), such as myoD with MRF4, and 4) nerve defector injury. Hence, it is possible that haploinsufficiency for elastin, LIM-kinasel, frizzled and/or syntaxin 1A may also contribute to the musculoskeletal defects in WBS. LIM-kinasel and syntaxin are involved in cytoskeletal organisation of the developing brain and nerve vesicular trafficking, while frizzled is involved in the development of body plan. However, atypical WBS patients with microdeletions of either LIMK-1 and STXIA do not exhibit musculoskeletal defects, while classical WBS patients with intact STXIA and FZD3 loci do exhibit musculoskeletal defects. Also, patients with deletions confined to the elastin gene locus exhibit only cardiovascular defects. Hence, it is unlikely that these genes cause the musculoskeletal defects. Yet it remains possible that in combination, as in classical WBS, they or any loci at the deletion point may act in concert with MusTRD to affect myofiber integrity and nerve-dependent signalling.

In summary, we have shown a myopathic phenotype induced in mice by a truncated MusTRD protein. The observed physical and muscular defects support the contention that MusTRD is an important regulator in myogenesis in at least three separate processes. Firstly, slow myofiber-specific genes are repressed in ΔMusTRD muscles, implicating MusTRD in regulating the slow myogenic phenotype. Loss of slow, fatigue-resistant myofibers may contribute to the altered posture and gait in these mice and may contribute to similar features in WBS patients. Secondly, ΔMusTRD is important for maturation of myofibers as evidenced by the retention of the developmental isoform of MHCₙₑₒ. Thirdly, disruption of MusTRD function results in reduced muscle mass and growth retardation, suggesting that MusTRD is required for growth hypertrophy of differentiated myotubes. These phenotypes are dose-sensitive and may mirror the effect of a Hemizygotic deletion of the MusTRD encoding gene. Furthermore, the growth defect in WBS, which was previously regarded as an underlying endocrine problem, can now be attributed in part to the disruption of the function of a muscle-specific transcription factor, MusTRD1.

All publications mentioned in the above specification are herein incorporated by reference. Various modifications and variations of the described methods and system of the invention will be apparent to those skilled in the art without departing from the scope of the invention. Although the invention has been described in connection with specific preferred embodiments, it should be understood that the invention as claimed should not be unduly limited to such specific embodiments. Indeed, various modifications of the described modes for carrying out the invention which are apparent to those skilled in molecular biology or related fields are intended to be within the scope of the invention.

References

Bayarsaihan, D. and Ruddle, F. H. 2000. Proc. Natl. Acad. Sci. USA 97: 7342-7347.
Borrione, A. C., Zanellato, A. M., Saggin, L., Mazzoli, M., Azzarello, G., and Sartore, S. 1988. Differentiation 38: 49-59.
Brennan, K. J. and Hardeman, E. C. 1993. J. Biol. Chem. 268: 719-725.
Calvo, S., Venepally, P., Cheng, J., and Buonanno, A. 1999. Mol. Cell Biol. 19: 515-525.
Corin, S. J., Levitt, L. K., O'Mahoney, J. V., Joya, J. E., and Hardeman, E.C. 1995. Proc. Natl. Acad. Sci. USA 92: 6185-6189.
di Maso N A, Haddad F, Zeng M, McCue S A. Baldwin K M. 2000. Journal of Applied Physiology 88(2):682-9.
Esser K, Nelson T, Lupa-Kimball V, Blough E. 1999. Journal of Biological Chemistry 274(17):12095-102.
Franke Y., Peoples R. J. and Francke U. 1999. Cytogenet Cell Genet 86: 296-304.
Horlein et al, 1995. Nature 377: 397-404.
Muscat, G. E. and Kades, L. 1987. Mol. Cell Biol. 7: 4089-4099.
O'Mahoney, J. V., Guven, K. L., Lin, J., Joya, J. E., Robinson, C. S., Wade, R. P., and Hardeman, E. C. 1998. Mol. Cell Biol. 18: 6641-6652.
Roy, A. L., Hong, D u., Gregor, P. D., Novina, C. D., Martinez, E., and Roeder, R. G. 1997. EMBO J. 16: 7091-7104.
Schiaffino, S., Gorza, L., Sartore, S., Saggin, L., Ausoni, S., Vianello, M., Gundersen, K., and Lomo, T. 1989. J. Muscle Res. Cell Motil. 10: 197-205.
Sutherland, C. J., Elsom, V. L., Gordon, M. L., Dunwoodie, S. L. and Hardeman, E. C. 1991. Dev. Biol. 146:167-178.
Umesono et al., 1991. Cell 65: 1255-1266.
Voit, T., Kramer, H., Thomas, C., Wechsler, W., Reichmann, H., and Lenard, H. G. 1991. Eur. J. Pediatr. 150: 521-526.
Wright C E. Bodell P W. Haddad F. Qin A X. Baldwin K M. 2001. American Journal of Physiology—Cell Physiology. 280(5):C1262-76.
Yan X., Zhao, X., Qian M., Gong N and Zhu X. Biochem J (2000) 345:749-757
Zhu, L., Lyons, G. E., Joya, J. E., Hardeman, E. C., and Wade, R. P. 1995. Dev. Biol. 169: 487-503.

### Sequence Listing Part of the Description

Mouse sequences (as shown in FIGS. 3 and 4).

| | | |
|---|---|---|
| 1alpha1 | SEQ ID No. 1 (nucleotide) | SEQ ID No. 2 (amino acid) |
| 1alpha4 | SEQ ID No. 3 (nucleotide) | SEQ ID No. 4 (amino acid) |
| 1beta1 | SEQ ID No. 5 (nucleotide) | SEQ ID No. 6 (amino acid) |
| 1beta4 | SEQ ID No. 7 (nucleotide) | SEQ ID No. 8 (amino acid) |
| 2alpha5 | SEQ ID No. 9 (nucleotide) | SEQ ID No. 10 (amino acid) |
| 3alpha3 | SEQ ID No. 11 (nucleotide) | SEQ ID No. 12 (amino acid) |
| 3beta3 | SEQ ID No. 13 (nucleotide) | SEQ ID No. 14 (amino acid) |
| 3alpha5 | SEQ ID No. 15 (nucleotide) | SEQ ID No. 16 (amino acid) |
| 3beta5 | SEQ ID No. 17 (nucleotide) | SEQ ID No. 18 (amino acid) |
| 3beta7 | SEQ ID No. 19 (nucleotide) | SEQ ID No. 20 (amino acid) |
| 3alpha7 | SEQ ID No. 21 (nucleotide) | SEQ ID No. 22 (amino acid) |

Human sequences (see FIG. 14).

| | |
|---|---|
| 1alpha1 | SEQ ID No. 23 (nucleotide) |
| 1alpha0 | SEQ ID No. 24 (nucleotide) |
| 1beta1 | SEQ ID No. 25 (nucleotide) |
| 1beta0 | SEQ ID No. 26 (nucleotide) |

| SEQ ID No. 27 - Exon 23 sequence of human MusTRD. | |
|---|---|
| GAACTGCCTCCTCACTTGGCTTCTCTCCCCCTGCCCTGCCCCCAGAGAGGGATTCCGGGG | |
| ACCCTCTGGTGGACGAGAGCCTGAAGAGACAGGGCTTTCAAG. | |
| SEQ ID No. 28 - Exon 30 sequence of human MusTRD | |
| AGTTGCGCAGAACAGGACCTGACCGCTCTTCCCTCTGGCTTTCAGCCGGCACTCGGGCAG | |
| GGTCGTCTACGCTGGGGTGTGGTCCAGGGTGCGGGGAGACGCCAGGTGCTGTGAGCAGGG | |
| TCTGCAGACTCTCCTGCCTGCCCACCCATGAGCTAGTCCACCTCTCCTCTCATCAGGT | |
| SEQ ID No. 29 - Exon 31 sequence of human MusTRD | |
| TGGCCAATGTACATGGTGGACTATGCCGGCCTGAACGTGCAGCTCCCGGGACCTCTTAAT | |
| TACTAGACCTCAGTACTGAATCAGGACCTCACTCAGAAAGACTAAAGGAAATGTAATTTA | |
| TGTACAAAATGTATATTCGGATATGTATCGATGCCTTTTAGTTTTTCCAATGATTTTTAC | |
| ACTATATTCCTGCCACCAAGGCCTTTTTAAATAAGT | |
| SEQ ID No. 30 Human MusTRD - amino acid sequence | |
| GenBank Accession No. XM_034686. | |
| 1 | taaatggcag ccaatggagg gtggtgttgc gcggggctgg gattagggcc ggggcgaatg |
| 61 | gctggcaatc ttactgggat tacagaacaa agagcctccc cgcgctcccg ctctccgctc |
| 121 | ctctccccgc gccgccccgc cctccgccgc agcccgcgcc gggggtgggg gccgccgagc |
| 181 | gccagccccc cggccggccg attccccccc cgcgccccct ccccgcgcct ccctccccgc |
| 241 | cctcgccgcg ccgccgtcct cgcctccctc tgcctctcct tcccccattc tcccggatta |
| 301 | attaaggagg cagcggcagg aggctgagtc ctggccgcgg gccggggccg gggcgccgct |
| 361 | ggcaggagcg cttggggatc ctccaaggcg accatggcct tgctgggtaa gcgctgtgac |
| 421 | gtccccacca acggctgcgg acccgaccgc tggaactccg cgttcacccg caaagacgag |
| 481 | atcatcacca gcctcgtgtc tgccttagac tccatgtgct cagcgctgtc caaactgaac |
| 541 | gccgaggtgg cctgtgtcgc cgtgcacgat gagagcgcct ttgtggtggg cacagagaag |
| 601 | gggagaatgt tcctgaatgc ccggaaggag ctacagtcag acttcctcag gttctgccga |
| 661 | gggcccccgt ggaaggatcc ggaggcagag caccccaaga aggtgcagcg gggcgagggt |
| 721 | ggaggccgta gcctccctcg gtcctccctg gaacatggct cagatgtgta ccttctgcgg |
| 781 | aagatggtag aggaggtgtt tgatgttctt tatagcgagg ccctgggaag ggccagtgtg |
| 841 | gtgccactgc cctatgagag gctgctcagg gagccagggc tgctggccgt gcaggggctg |
| 901 | cccgaaggcc tggccttccg aaggccagcc gagtatgacc ccaaggccct catggccatc |
| 961 | ctggaacaca gccaccgcat ccgcttcaag ctcaagaggc cacttgagga tggcgggcgg |
| 1021 | gactcgaagg ccctggtgga gctgaacggt gtctccctga ttcccaaggg gtcacgggac |
| 1081 | tgtggcctgc atggccaggc ccccaaggtg ccaccccagg acctgccccc aaccgccacc |
| 1141 | tcctcctcca tggccagctt cctgtacagc acggcgctcc ccaaccacgc catccgagag |
| 1201 | ctcaagcagg aagcaccttc ctgccccctt gcccccagcg acctgggcct gagtcggccc |
| 1261 | atgccagagc ccaaggccac cggtgcccaa gacttctccg actgttgtgg acagaagccc |
| 1321 | actgggcctg gtgggcctct catccagaac gtccatgcct ccaagcgcat tctcttctcc |
| 1381 | atcgtccatg acaagtcaga gaagtgggac gccttcataa aggaaaccga ggacatcaac |
| 1441 | acgctccggg agtgtgtgca gatcctgttt aacagcagat atgcggaagc cctgggcctg |
| 1501 | gaccacatgg tccccgtgcc ctaccggaag attgcctgtg acccggaggc tgtggagatc |
| 1561 | gtgggcatcc cggacaagat ccccttcaag cgcccctgca cttatggagt ccccaagctg |
| 1621 | aagcggatcc tggaggagcg ccatagtatc cacttcatca ttaagaggat gtttgatgag |
| 1681 | cgaattttca cagggaacaa gtttaccaaa gacaccacga agctggagcc agccagcccg |
| 1741 | ccagaggaca cctctgcaga ggtctctagg gccaccgtcc ttgaccttgc tgggaatgct |
| 1801 | cggtcagaca agggcagcat gtctgaagac tgtgggccag gaacctccgg ggagctgggc |
| 1861 | gggctgaggc cgatcaaaat tgagccagag gatctggaca tcattcaggt caccgtccca |
| 1921 | gacccctcgc caacctctga ggaaatgaca gactcgatgc ctgggcacct gccatcggag |
| 1981 | gattctggtt atgggatgga gatgctgaca gacaaaggtc tgagtgagga cgcgcggccc |
| 2041 | gaggagaggc ccgtggagga cagccacggt gacgtgatcc ggcccctgcg gaagcaggtg |
| 2101 | gagctgctct tcaacacacg atacgccaag gccattggca tctcggagcc cgtcaaggtg |
| 2161 | ccgtactcca agtttctgat gcacccggag gagctgtttg tggtgggact gcctgaaggc |
| 2221 | atctccctcc gcaggcccaa ctgcttcggg atcgccaagc tccggaagat tctggaggcc |
| 2281 | agcaacagca tccagtttgt catcaagagg cccgagctgc tcactgaggg agtcaaagag |
| 2341 | cccatcatgg atagtcaagg aactgcctcc tcacttggct tctctccccc tgccctgccc |
| 2401 | ccagagaggg attccgggga ccctctggtg gacgagagcc tgaagagaca gggctttcaa |
| 2461 | gaaaattatg acgcgaggct ctcacggatc gacatcgcca acacactaag ggagcaggtc |
| 2521 | caggaccttt tcaataagaa atacggggaa gccttgggca tcaagtaccc ggtccaggtc |
| 2581 | ccctacaagc ggatcaagag taaccccggc tccgtgatca tcgaggggct gcccccagga |
| 2641 | atcccgttcc gaaagccctg taccttcggc tcccagaacc tggagaggat tcttgctgtg |
| 2701 | gctgacaaga tcaagttcac agtcaccagg cctttccaag gactcatccc aaagcctgat |
| 2761 | gaagatgacg ccaacagact cggggagaag gtgatcctgc gggagcaggt gaaggaactc |
| 2821 | ttcaacgaga aatacggtga ggccctgggc ctgaaccggc cggtgctggt cccttataaa |
| 2881 | ctaatccggg acagcccaga cgccgtggag gtcacgggtc tgcctgatga catccccttc |
| 2941 | cggaacccca acacgtacga catccaccgg ctggagaaga tcctgaaggc ccgagagcat |
| 3001 | gtccgcatgg tcatcattaa ccagctccaa ccctttgcag aaatctgcaa tgatgccaag |
| 3061 | gtgccagcca aagacagcag cattcccaag cgcaagagaa agcgggtctc ggaaggaaat |
| 3121 | tccgtctcct cttcctcctc gtcttcctct tcctcgtcct ctaacccgga ttcagtggca |
| 3181 | tcggccaacc agatctcact cgtgcaatgg ccaatgtaca tggtggacta tgccggcctg |
| 3241 | aacgtgcagc tcccgggacc tcttaattac tagacctcag tactgaatca ggacctcact |
| 3301 | cagaaagact aaaggaaatg taatttatgt acaaaatgta tattcggata tgtatcgatg |
| 3361 | ccttttagtt tttccaatga tttttacact atattcctgc caccaaggcc tttttaaata |
| 3421 | agt |

| SEQ ID No. 31 Human MusTRD - amino acid sequence | |
|---|---|
| GenBank Accession No. XM_034686. | |
| MALLGKRCDVPTNGCGPDRWNSAFTRKDEI | 30 |
| ITSLVSALDSMCSALSKLNAEVACVAVHDE | 60 |
| SAFVVGTEKGRMFLNARKELQSDFLRFCRG | 90 |
| PPWKDPEAEHPKKVQRGEGGGRSLPRSSLE | 120 |
| HGSDVYLLRKMVEEVFDVLYSEALGRASVV | 150 |
| PLPYERLLREPGLLAVQGLPEGLAFRRPAE | 180 |
| YDPKALMAILEHSHRIRFKLKRPLEDGGRD | 210 |
| SKALVELNGVSLIPKGSRDCGLHGQAPKVP | 240 |
| PQDLPPTATSSSMASFLYSTALPNHAIREL | 270 |
| KQEAPSCPLAPSDLGLSRPMPEPKATGAQD | 300 |
| FSDCCGQKPTGPGGPLIQNVHASKRILFSI | 330 |
| VHDKSEKWDAFIKETEDINTLRECVQILFN | 360 |
| SRYAEALGLDHMVPVPYRKIACDPEAVEIV | 390 |
| GIPDKIPFKRPCTYGVPKLKRILEERHSIH | 420 |
| FIIKRMFDERIFTGNKFTKDTTKLEPASPP | 450 |
| EDTSAEVSRATVLDLAGNARSDKGSMSEDC | 480 |
| GPGTSGELGGLRPIKIEPEDLDIIQVTVPD | 510 |
| PSPTSEEMTDSMPGHLPSEDSGYGMEMLTD | 540 |
| KGLSEDARPEERPVEDSHGDVIRPLRKQVE | 570 |
| LLFNTRYAKAIGISEPVKVPYSKFLMHPEE | 600 |
| LFVVGLPEGISLRRPNCFGIAKLRKILEAS | 630 |
| NSIQFVIKRPELLTEGVKEPIMDSQGTASS | 660 |
| LGFSPPALPPERDSGDPLVDESLKRQGFQE | 690 |
| NYDARLSRIDIANTLREQVQDLFNKKYGEA | 720 |
| LGIKYPVQVPYKRIKSNPGSVIIEGLPPGI | 750 |
| PFRKPCTFGSQNLERILAVADKIKFTVTRP | 780 |
| FQGLIPKPDEDDANRLGEKVILREQVKELF | 810 |
| NEKYGEALGLNRPVLVPYKLIRDSPDAVEV | 840 |
| TGLPDDIPFRNPNTYDIHRLEKILKAREHV | 870 |
| RMVIINQLQPFAEICNDAKVPAKDSSIPKR | 900 |
| KRKRVSEGNSVSSSSSSSSSSSSNPDSVAS | 930 |
| ANQISLVQWPMYMVDYAGLNVQLPGPLNY | 959 |

The invention will now be further described by the following numbered paragraphs:

1. A method of modulating the relative composition of slow and fast myofibres in muscle tissue of a human or animal which method comprises modulating in myogenic cells of the human or animal the levels and/or activity of MusTRD.

2. A method of modulating the relative composition of slow and fast myofibres in muscle tissue of a human or animal which method comprises administering to the human or animal a compound capable of modulating the levels and/or activity of MusTRD in myogenic cells of the human or animal.

3. A method according to paragraph 2 wherein the compound is a MusTRD1α1 polypeptide or an isoform or fragment thereof, or a nucleic acid encoding said compound.

4. A method of modulating the amount of slow and/or fast myofibres in muscle tissue of a human or animal which method comprises modulating in myogenic cells of the human or animal the levels and/or activity of MusTRD.

5. A method of modulating the amount of slow and/or fast myofibres in muscle tissue of a human or animal which method comprises administering to the human or animal a compound capable of modulating the levels and/or activity of MusTRD in myogenic cells of the human or animal.

6. A method according to paragraph 5 wherein the compound is a MusTRD1 α1 polypeptide or an isoform or fragment thereof, or a nucleic acid encoding said compound.

7. A method of regulating myofibre specialisation in a human or animal which method comprises modulating in myogenic cells of the human or animal the levels and/or activity of MusTRD.

8. A method of regulating myofibre specialisation in a human or animal which method comprises administering to the human or animal a compound capable of modulating the levels and/or activity of MusTRD in myogenic cells of the human or animal.

9. A method according to paragraph 8 wherein the compound is a MusTRD1 α1 polypeptide or an isoform or fragment thereof, or a nucleic acid encoding said compound.

10. A method of treating a disease or condition characterised by muscular defects which method comprises administering to the human or animal a compound capable of modulating the, levels and/or activity of MusTRD in myogenic cells of the human or animal.

11. A method according to paragraph 10 wherein the compound is a MusTRD1α1 polypeptide or an isoform or fragment thereof, or a nucleic acid encoding said compound.

12. A method according to paragraph 10 or paragraph 11 wherein the muscular defects are abnormal myofibre composition, abnormal myofibre maturation and/or abnormal growth hypertrophy of differentiated myotubes.

13. A method of regulating expression of a myosin light chain 1 slowA (MLC1_{slowA}), a-tropomyosin slow (α-Tm_{slow}), myosin heavy chain type I (MHC I), and/or troponin I slow (Tnl_{slow}) polypeptide in a cell which method comprises administering to/expressing in said cell a MusTRD polypeptide or fragment thereof.

14. A polypeptide comprising a MusTRD polypeptide or fragment thereof for use in therapy.

15. A polypeptide comprising a MusTRD polypeptide or fragment thereof for use in modulating the relative composition of slow and fast myofibres in muscle tissue of a human or animal.

16. A polypeptide comprising a MusTRD polypeptide or fragment thereof for use in modulating the amount of slow and/or fast myofibres in muscle tissue of a human or animal.

17. A polypeptide comprising a MusTRD polypeptide or fragment thereof for use in regulating myofibre specialisation in a human or animal.

18. A polynucleotide encoding a MusTRD polypeptide or fragment thereof for use in therapy.

19. A polynucleotide encoding a MusTRD polypeptide or fragment thereof for use in modulating the relative composition of slow and fast myofibres in muscle tissue of a human or animal.

20. A polynucleotide encoding a MusTRD polypeptide or fragment thereof for use in modulating the amount of slow and/or fast myofibres in muscle tissue of a human or animal.

21. A polynucleotide encoding a MusTRD polypeptide or fragment thereof for use in regulating myofibre specialisation in a human or animal.

22. A polynucleotide encoding a MusTRD polypeptide or fragment thereof for use in treating muscular defects.

23. A polypeptide comprising the amino acid sequence shown in any one of SEQ. Nos. 2, 4, 6, 8, 10, 12 and 14 or an orthologue thereof, or a fragment thereof comprising a Box 5 region.

24. A polypeptide according to paragraph 23 wherein said fragment comprises the transcriptional activation/repression domain of the full length polypeptide.

25. A polynucleotide encoding a polypeptide according to paragraph 23.

26. A polynucleotide selected from the group consisting of:
(a) polynucleotides having the sequence as shown in any one of SEQ ID Nos. 1, 37 51 71 91 11, 13, 15, 17 and 19 and orthologues thereof;
(b) fragments of the polynucleotides of (a) comprising a sequence encoding a Box 5 region and/or an RD5 region;
(c) fragments of the polynucleotides of (a) comprising a sequence encoding a DBDL domain and/or a DBD2 domain;
(d) polynucleotides which are degenerate as a result of the genetic code to any of the polynucleotides of (a), (b) or (c); and
(e) polynucleotides which are complementary to the polynucleotides of (a), (b), (c) or (d);
with the proviso that the full length human CREAM-1 nucleotide sequence , the full length.human WBSCR11 nucleotide sequence, the full length human GTF21 RD1 nucleotide sequence and the full length human GTF3 nucleotide sequence are specifically excluded.

27. A nucleic acid vector comprising a polynucleotide according to paragraph 25 or paragraph 26.

28. A host cell comprising a polynucleotide according to paragraph 25 or paragraph 26 and/or a nucleic acid vector according to paragraph 27.

29. A method of producing a polypeptide according to paragraph 23 or paragraph 24 which comprises culturing a host cell according to paragraph 28 under conditions that allow for expression of said polypeptide in said cell.

30. A nucleotide probe/primer comprising at least 15 nucleotides which hybridises specifically to a MusTRD polynucleotide sequence selected from exons 19, 21, 22, 23, 26, 27, 30 and 31 of a mouse MusTRD isoform, or the equivalent region of an orthologue thereof.

31. A nucleotide probe/primer comprising at least 15 nucleotides which hybridises specifically to a MusTRD polynucleotide selected from a box 5 region and an RD5 region.

32. A method of identifying the presence of a MusTRD isoform in a sample which method comprises determining the presence in the sample of one or more nucleotide regions selected from exons 19, 21, 22, 23, 26, 27, 30 and 31 of a mouse MusTRD isoform, or the equivalent region of an orthologue thereof.

33. A method according to paragraph 32 wherein the presence of the one or more nucleotide regions is determined by nucleic acid amplification using one or more probes/primers as defined in paragraph 30 or paragraph 31.

34. An antibody that binds specifically to a MusTRD polypeptide according to paragraph 23 or paragraph 24.

35. An antibody according to paragraph 34 which binds specifically to a Box 5 region or an RD5 region of a MusTRD polypeptide.

36. A method of identifying the presence of a MusTRD isoform in a sample which method comprises:
(a) providing an antibody according to paragraph 34;
(b) incubating the sample with said antibody under conditions which allow for the formation of an antibody-antigen complex; and
(c) determining whether an antibody-antigen complex comprising said antibody is formed.

1. A method of modulating the relative composition of slow and fast myofibres or of modulating the amount of slow and/or fast myofibres, in muscle tissue of a human or animal, or of regulating myofibre specialization in a human or animal, which method comprises modulating in myogenic cells of the human or animal the levels and/or activity of MusTRD.

2. The method of claim 1, wherein said method comprises administering to the human or animal a compound capable of modulating the levels and/or activity of MusTRD in myogenic cells of the human or animal.

3. The method according to claim 2 wherein the compound is a MusTRD1α1 polypeptide or an isoform or fragment thereof, or a nucleic acid encoding said compound.

4. A method of treating a disease or condition characterised by muscular defects which method comprises administering to the human or animal a compound capable of modulating the levels and/or activity of MusTRD in myogenic cells of the human or animal.

5. The method according to claim 4 wherein the compound is a MusTRD1α1 polypeptide or an isoform or fragment thereof, or a nucleic acid encoding said compound.

6. The method according to claim 4 wherein the muscular defects are abnormal myofibre composition, abnormal myofibre maturation and/or abnormal growth hypertrophy of differentiated myotubes.

7. A method of regulating expression of a myosin light chain 1 slowA (MLC1_{slowA}), α-tropomyosin slow (α-Tm_{slow}), myosin heavy chain type I (MHC I), and/or troponin I slow (TnI_{slow}) polypeptide in a cell which method comprises administering to/expressing in said cell a MusTRD polypeptide or fragment thereof.

8. A polypeptide comprising a MusTRD polypeptide or fragment thereof, or a polynucleotide encoding a MusTRD polypeptide or fragment thereof, for use in therapy.

9. The polypeptide or fragment of claim 8, wherein the polypeptide or fragment is for use in modulating the relative composition of slow and fast myofibres in muscle tissue of a human or animal.

10. The polypeptide or fragment of claim 8, wherein the polypeptide or fragment is for use in modulating the amount of slow and/or fast myofibres in muscle tissue of a human or animal.

11. The polypeptide or fragment of claim 8, wherein the polypeptide or fragment is for use in regulating myofibre specialisation in a human or animal.

12. The polynucleotide of claim 8, wherein the encoded polypeptide or fragment is for use in modulating the relative composition of slow and fast myofibres in muscle tissue of a human or animal.

13. The polynucleotide of claim 8, wherein the encoded polypeptide or fragment is for use in modulating the amount of slow and/or fast myofibres in muscle tissue of a human or animal.

14. The polynucleotide of claim 8, wherein the encoded polypeptide or fragment is for use in regulating myofibre specialisation in a human or animal.

15. The polynucleotide of claim 8, wherein the encoded polypeptide or fragment is for use in treating muscular defects.

16. A polypeptide comprising the amino acid sequence shown in any one of SEQ. Nos. 2, 4, 6, 8, 10, 12 and 14 or an orthologue thereof, or a fragment thereof comprising a Box 5 region.

17. A polypeptide according to claim 16 wherein said fragment comprises the transcriptional activation/repression domain of the full length polypeptide.

18. A polynucleotide encoding a polypeptide according to claim 16.

19. A polynucleotide selected from the group consisting of:
(a) polynucleotides having the sequence as shown in any one of SEQ ID Nos. 1, 3, 5, 7, 9, 11, 13, 15, 17 and 19 and orthologues thereof;
(b) fragments of the polynucleotides of (a) comprising a sequence encoding a Box 5 region and/or an RD5 region;
(c) fragments of the polynucleotides of (a) comprising a sequence encoding a DBD1 domain and/or a DBD2 domain;
(d) polynucleotides which are degenerate as a result of the genetic code to any of the polynucleotides of (a), (b) or (c); and
(e) polynucleotides which are complementary to the polynucleotides of (a), (b), (c) or (d);
with the proviso that the full length human CREAM-1 nucleotide sequence, the full length human WBSCR11 nucleotide sequence, the full length human GTF21RD1 nucleotide sequence and the full length human GTF3 nucleotide sequence are specifically excluded.

20. A nucleic acid vector comprising a polynucleotide according to claim 18.

21. A nucleic acid vector comprising a polynucleotide according to claim 19.

22. A host cell comprising a polynucleotide according to claim 18 and/or a nucleic acid vector comprising a polynucleotide according to claim 18.

23. A host cell comprising a polynucleotide according to claim 19 and/or a nucleic acid vector according to claim 19.

24. A method of producing a polypeptide comprising the amino acid sequence shown in any one of SEQ. Nos. 2, 4, 6, 8, 10, 12 and 14 or an orthologue thereof, or a fragment thereof comprising a Box 5 region, which comprises culturing a host cell according to claim 22 under conditions that allow for expression of said polypeptide in said cell.

25. A method of producing a polypeptide comprising the amino acid sequence shown in any one of SEQ. Nos. 2, 4, 6, 8, 10, 12 and 14 or an orthologue thereof, or a fragment thereof comprising a Box 5 region, which comprises culturing a host cell according to claim 23 under conditions that allow for expression of said polypeptide in said cell.

26. A nucleotide probe/primer comprising at least 15 nucleotides which hybridises specifically to a MusTRD polynucleotide sequence selected from exons 19, 21, 22, 23, 26, 27, 30 and 31 of a mouse MusTRD isoform, or the equivalent region of an orthologue thereof.

27. A nucleotide probe/primer comprising at least 15 nucleotides which hybridises specifically to a MusTRD polynucleotide selected from a box 5 region and an RD5 region.

28. A method of identifying the presence of a MusTRD isoform in a sample which method comprises determining the presence in the sample of one or more nucleotide regions selected from exons 19, 21, 22, 23, 26, 27, 30 and 31 of a mouse MusTRD isoform, or the equivalent region of an orthologue thereof.

29. A method according to claim 28 wherein the presence of the one or more nucleotide regions is determined by nucleic acid amplification using one or more probes/primers comprising at least 15 nucleotides which hybridises specifically to a MusTRD polynucleotide sequence selected from exons 19, 21, 22, 23, 26, 27, 30 and 31 of a mouse MusTRD isoform, or the equivalent region of an orthologue thereof.

30. An antibody that binds specifically to a MusTRD polypeptide according to claim 16.

31. An antibody according to claim 30 which binds specifically to a Box 5 region or an RD5 region of a MusTRD polypeptide.

32. A method of identifying the presence of a MusTRD isoform in a sample which method comprises:
(a) providing an antibody according to claim 30;
(b) incubating the sample with said antibody under conditions which allow for the formation of an antibody-antigen complex; and
(c) determining whether an antibody-antigen complex comprising said antibody is formed.
